SEQUENCE LISTING (120 sequences)

SEQ ID NO: 1 - protein, 112 aa, synthetic construct
DIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIK

SEQ ID NO: 2 - protein, 16 aa, synthetic construct
KSSQSLVHNNANTYLS

SEQ ID NO: 3 - protein, 7 aa, synthetic construct
KVSNRFS

SEQ ID NO: 4 - protein, 9 aa, synthetic construct
GQGTQYPFT

SEQ ID NO: 5 - protein, 121 aa, synthetic construct
QVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSS

SEQ ID NO: 6 - protein, 5 aa, synthetic construct
KAWMH

SEQ ID NO: 7 - protein, 19 aa, synthetic construct
QIKDKSNSYATYYADSVKG

SEQ ID NO: 8 - protein, 12 aa, synthetic construct
RGVYYALSPFDY

SEQ ID NO: 9 - protein, 112 aa, synthetic construct
DIVMTQSPLSLPVTPGEPASISCRSSQSLVHNQANTYLSWYLQKPGQSPQSLIYKVSNRFSGVPDRFSGSGSGTDFTLKISRVEAEDVGVYYCGQGTQYPFTFGSGTKVEIK

SEQ ID NO: 10 - protein, 16 aa, synthetic construct
RSSQSLVHNQANTYLS

SEQ ID NO: 11 - protein, 7 aa, synthetic construct
KVSNRFS

SEQ ID NO: 12 - protein, 9 aa, synthetic construct
GQGTQYPFT

SEQ ID NO: 13 - protein, 112 aa, synthetic construct
DIVMTQSPLSLPVTPGEPASISCRSSQSLVHNTGNTYLSWYLQKPGQSPQSLIYKVSNRASGVPDRFSGSGSGTDFTLKISRVEAEDVGVYYCGQGTQYPFTFGSGTKVEIK

SEQ ID NO: 14 - protein, 16 aa, synthetic construct
RSSQSLVHNTGNTYLS

SEQ ID NO: 15 - protein, 7 aa, synthetic construct
KVSNRAS

SEQ ID NO: 16 - protein, 9 aa, synthetic construct
GQGTQYPFT

SEQ ID NO: 17 - protein, 112 aa, synthetic construct
DIVMTQSPLSLPVTPGEPASISCRSSQSLVHNQANTYLSWYLQKPGQSPQSLIYKVSNRYSGVPDRFSGSGSGTDFTLKISRVEAEDVGVYYCGQGTQYPFTFGSGTKVEIK

SEQ ID NO: 18 - protein, 16 aa, synthetic construct
RSSQSLVHNQANTYLS

SEQ ID NO: 19 - protein, 7 aa, synthetic construct
KVSNRYS

SEQ ID NO: 20 - protein, 9 aa, synthetic construct
GQGTQYPFT

SEQ ID NO: 21 - protein, 121 aa, synthetic construct
EVQLVESGGALVKPGGSLRLSCAASGFTFTKAWMHWVRQAPGKQLEWVAQIKDKSNSYATYYAESVKGRFTISRDDSKNTLYLQMNSLKTEDTAVYYCRGVYYALSPFDYWGQGTLVTVSS

SEQ ID NO: 22 - protein, 5 aa, synthetic construct
KAWMH

SEQ ID NO: 23 - protein, 20 aa, synthetic construct
QIKDKSNSYATYYAESVKGR

SEQ ID NO: 24 - protein, 12 aa, synthetic construct
RGVYYALSPFDY

SEQ ID NO: 25 - protein, 121 aa, synthetic construct
EVQLVESGGALVKPGGSLRLSCAASGFTFTKAYMHWVRQAPGKQLEWVAQIKDKSNSYATYYAESVKGRFTISRDDSKNTLYLQMNSLKTEDTAVYYCRGVYYALSPFDYWGQGTLVTVSS

SEQ ID NO: 26 - protein, 5 aa, synthetic construct
KAYMH

SEQ ID NO: 27 - protein, 20 aa, synthetic construct
QIKDKSNSYATYYAESVKGR

SEQ ID NO: 28 - protein, 12 aa, synthetic construct
RGVYYALSPFDY

SEQ ID NO: 29 - protein, 121 aa, synthetic construct
EVQLVESGGALVKPGGSLRLSCAASGFTFSNAWMHWVRQAPGKQLEWVAQIKDKSNSYATYYAESVKGRFTISRDDSKNTLYLQMNSLKTEDTAVYYCRGVYYALSPFDYWGQGTLVTVSS

SEQ ID NO: 30 - protein, 5 aa, synthetic construct
NAWMH

SEQ ID NO: 31 - protein, 20 aa, synthetic construct
QIKDKSNSYATYYAESVKGR

SEQ ID NO: 32 - protein, 12 aa, synthetic construct
RGVYYALSPFDY

SEQ ID NO: 33 - protein, 121 aa, synthetic construct
EVQLVESGGALVKPGGSLRLSCAASGFTFTKAWMSWVRQAPGKQLEWVAQIKDKSNSYATYYAESVKGRFTISRDDSKNTLYLQMNSLKTEDTAVYYCRGVYYALSPFDYWGQGTLVTVSS

SEQ ID NO: 34 - protein, 5 aa, synthetic construct
KAWMS

SEQ ID NO: 35 - protein, 20 aa, synthetic construct
QIKDKSNSYATYYAESVKGR

SEQ ID NO: 36 - protein, 12 aa, synthetic construct
RGVYYALSPFDY

SEQ ID NO: 37 - protein, 121 aa, synthetic construct
EVQLVESGGALVKPGGSLRLSCAASGFTFTKAYMHWVRQAPGKQLEWVAQIKDRSQSYATYYAESVKGRFTISRDDSKNTLYLQMNSLKTEDTAVYYCRGVYYALSPFDYWGQGTLVTVSS

SEQ ID NO: 38 - protein, 5 aa, synthetic construct
KAYMH

SEQ ID NO: 39 - protein, 20 aa, synthetic construct
QIKDRSQSYATYYAESVKGR

SEQ ID NO: 40 - protein, 12 aa, synthetic construct
RGVYYALSPFDY

SEQ ID NO: 41 - protein, 121 aa, synthetic construct
EVQLVESGGALVKPGGSLRLSCAASGFTFSNAYMHWVRQAPGKQLEWVAQIKDRSQSYATYYAESVKGRFTISRDDSKNTLYLQMNSLKTEDTAVYYCRGVYYALSPFDYWGQGTLVTVSS

SEQ ID NO: 42 - protein, 5 aa, synthetic construct
NAYMH

SEQ ID NO: 43 - protein, 20 aa, synthetic construct
QIKDRSQSYATYYAESVKGR

SEQ ID NO: 44 - protein, 12 aa, synthetic construct
RGVYYALSPFDY

SEQ ID NO: 45 - protein, 112 aa, synthetic construct
DIVMTQSPLSLPVTPGEPASISCRSSQSLVHNTGNTYLSWYLQKPGQSPQSLIYKVSNRASGVPDRFSGSGSGTDFTLKISRVEAEDVGVYYCGQGTQAPFTFGSGTKVEIK

SEQ ID NO: 46 - protein, 16 aa, synthetic construct
RSSQSLVHNTGNTYLS

SEQ ID NO: 47 - protein, 7 aa, synthetic construct
KVSNRAS

SEQ ID NO: 48 - protein, 9 aa, synthetic construct
GQGTQAPFT

SEQ ID NO: 49 - protein, 121 aa, synthetic construct
EVQLVESGGALVKPGGSLRLSCAASGFTFSNAWMHWVRQAPGKQLEWVAQIKDRSQTYATYYAESVKGRFTISRDDSKNTLYLQMNSLKTEDTAVYYCRGVYYALAPFDYWGQGTLVTVSS

SEQ ID NO: 50 - protein, 5 aa, synthetic construct
NAWMH

SEQ ID NO: 51 - protein, 20 aa, synthetic construct
QIKDRSQTYATYYAESVKGR

SEQ ID NO: 52 - protein, 12 aa, synthetic construct
RGVYYALAPFDY

SEQ ID NO: 53 - protein, 121 aa, synthetic construct
EVQLVESGGALVKPGGSLRLSCAASGFTFTKAYMHWVRQAPGKQLEWVAQIKDRSQSYATYYAESVKGRFTISRDDSKNTLYLQMNSLKTEDTAVYYCRGVYYALAPFDYWGQGTLVTVSS

SEQ ID NO: 54 - protein, 5 aa, synthetic construct
KAYMH

SEQ ID NO: 55 - protein, 8 aa, synthetic construct
QIKDRSQS

SEQ ID NO: 56 - protein, 12 aa, synthetic construct
RGVYYALAPFDY

SEQ ID NO: 57 - protein, 112 aa, synthetic construct
DIVMTQSPLSLPVTPGEPASISCRSSQSLVHNTGNTYLSWYLQKPGQSPQSLIYKVSNRASGVPDRFSGSGSGTDFTLKISRVEAEDVGVYYCGQGTQAPFSFGSGTKVEIK

SEQ ID NO: 58 - protein, 16 aa, synthetic construct
RSSQSLVHNTGNTYLS

SEQ ID NO: 59 - protein, 7 aa, synthetic construct
KVSNRAS

SEQ ID NO: 60 - protein, 9 aa, synthetic construct
GQGTQAPFS

SEQ ID NO: 61 - protein, 112 aa, synthetic construct
DIVMTQSPLSLPVTPGEPASISCRSSQSLVHNTGNTYLSWYLQKPGQSPQSLIYKVSNRASGVPDRFSGSGSGTDFTLKISRVEAEDVGVYYCGQGTQFPFTFGSGTKVEIK

SEQ ID NO: 62 - protein, 16 aa, synthetic construct
RSSQSLVHNTGNTYLS

SEQ ID NO: 63 - protein, 7 aa, synthetic construct
KVSNRAS

SEQ ID NO: 64 - protein, 9 aa, synthetic construct
GQGTQFPFT

SEQ ID NO: 65 - protein, 121 aa, synthetic construct
EVQLVESGGALVKPGGSLRLSCAASGFTFTRAWMHWVRQAPGKQLEWVAQIKDRSQTYATYYAESVKGRFTISRDDSKNTLYLQMNSLKTEDTAVYYCRGVYYANAPFDYWGQGTLVTVSS

SEQ ID NO: 66 - protein, 5 aa, synthetic construct
RAWMH

SEQ ID NO: 67 - protein, 20 aa, synthetic construct
QIKDRSQTYATYYAESVKGR

SEQ ID NO: 68 - protein, 12 aa, synthetic construct
RGVYYANAPFDY

SEQ ID NO: 69 - protein, 121 aa, synthetic construct
EVQLVESGGALVKPGGSLRLSCAASGFTFTRAWMHWVRQAPGKQLEWVAQIKDRSQTYATYYAESVKGRFTISRDDSKNTLYLQMNSLKTEDTAVYYCRGVYYAVTPFDYWGQGTLVTVSS

SEQ ID NO: 70 - protein, 5 aa, synthetic construct
RAWMH

SEQ ID NO: 71 - protein, 20 aa, synthetic construct
QIKDRSQTYATYYAESVKGR

SEQ ID NO: 72 - protein, 12 aa, synthetic construct
RGVYYAVTPFDY

SEQ ID NO: 73 - protein, 121 aa, synthetic construct
EVQLVESGGALVKPGGSLRLSCAASGFTFTRAWMHWVRQAPGKQLEWVAQIKDRSQTYATYYAESVKGRFTISRDDSKNTLYLQMNSLKTEDTAVYYCRGVYYANTPFDYWGQGTLVTVSS

SEQ ID NO: 74 - protein, 5 aa, synthetic construct
RAWMH

SEQ ID NO: 75 - protein, 20 aa, synthetic construct
QIKDRSQTYATYYAESVKGR

SEQ ID NO: 76 - protein, 12 aa, synthetic construct
RGVYYANTPFDY

SEQ ID NO: 77 - protein, 121 aa, synthetic construct
EVQLVESGGALVKPGGSLRLSCAASGFTFSNAWMHWVRQAPGKQLEWVAQIKDRSQTYATYYAESVKGRFTISRDDSKNTLYLQMNSLKTEDTAVYYCRGVYYANAPFDYWGQGTLVTVSS

SEQ ID NO: 78 - protein, 5 aa, synthetic construct
NAWMH

SEQ ID NO: 79 - protein, 20 aa, synthetic construct
QIKDRSQTYATYYAESVKGR

SEQ ID NO: 80 - protein, 12 aa, synthetic construct
RGVYYANAPFDY

SEQ ID NO: 81 - protein, 121 aa, synthetic construct
EVQLVESGGALVKPGGSLRLSCAASGFTFSNAWMHWVRQAPGKQLEWVAQIKDRSQTYATYYAESVKGRFTISRDDSKNTLYLQMNSLKTEDTAVYYCRGVYYAVTPFDYWGQGTLVTVSS

SEQ ID NO: 82 - protein, 5 aa, synthetic construct
NAWMH

SEQ ID NO: 83 - protein, 20 aa, synthetic construct
QIKDRSQTYATYYAESVKGR

SEQ ID NO: 84 - protein, 12 aa, synthetic construct
RGVYYAVTPFDY

SEQ ID NO: 85 - protein, 121 aa, synthetic construct
EVQLVESGGALVKPGGSLRLSCAASGFTFSNAWMHWVRQAPGKQLEWVAQIKDRSQTYATYYAESVKGRFTISRDDSKNTLYLQMNSLKTEDTAVYYCRGVYYANTPFDYWGQGTLVTVSS

SEQ ID NO: 86 - protein, 5 aa, synthetic construct
NAWMH

SEQ ID NO: 87 - protein, 20 aa, synthetic construct
QIKDRSQTYATYYAESVKGR

SEQ ID NO: 88 - protein, 12 aa, synthetic construct
RGVYYANTPFDY

SEQ ID NO: 89 - protein, 121 aa, synthetic construct
EVQLVESGGALVKPGGSLRLSCAASGFTFTEAWMHWVRQAPGKQLEWVAQIKDRSQTYATYYAESVKGRFTISRDDSKNTLYLQMNSLKTEDTAVYYCRGVYYANAPFDYWGQGTLVTVSS

SEQ ID NO: 90 - protein, 5 aa, synthetic construct
EAWMH

SEQ ID NO: 91 - protein, 20 aa, synthetic construct
QIKDRSQTYATYYAESVKGR

SEQ ID NO: 92 - protein, 12 aa, synthetic construct
RGVYYANAPFDY

SEQ ID NO: 93 - protein, 121 aa, synthetic construct
EVQLVESGGALVKPGGSLRLSCAASGFTFTEAWMHWVRQAPGKQLEWVAQIKDRSQTYATYYAESVKGRFTISRDDSKNTLYLQMNSLKTEDTAVYYCRGVYYAVTPFDYWGQGTLVTVSS

SEQ ID NO: 94 - protein, 5 aa, synthetic construct
EAWMH

SEQ ID NO: 95 - protein, 20 aa, synthetic construct
QIKDRSQTYATYYAESVKGR

SEQ ID NO: 96 - protein, 12 aa, synthetic construct
RGVYYAVTPFDY

SEQ ID NO: 97 - protein, 121 aa, synthetic construct
EVQLVESGGALVKPGGSLRLSCAASGFTFTEAWMHWVRQAPGKQLEWVAQIKDRSQTYATYYAESVKGRFTISRDDSKNTLYLQMNSLKTEDTAVYYCRGVYYANTPFDYWGQGTLVTVSS

SEQ ID NO: 98 - protein, 5 aa, synthetic construct
EAWMH

SEQ ID NO: 99 - protein, 20 aa, synthetic construct
QIKDRSQTYATYYAESVKGR

SEQ ID NO: 100 - protein, 12 aa, synthetic construct
RGVYYANTPFDY

SEQ ID NO: 101 - protein, 103 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 102 - protein, 104 aa, Homo sapiens
LLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTIS

SEQ ID NO: 103 - protein, 107 aa, Homo sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 106 - protein, 107 aa, Homo sapiens [sequence omitted]

SEQ ID NO: 107 - protein, 106 aa, synthetic construct
GQPKAAPSVTLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 108 - protein, 103 aa, synthetic construct
ASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSV

SEQ ID NO: 109 - protein, 6 aa, synthetic construct
DDYAIG

SEQ ID NO: 110 - protein, 17 aa, synthetic construct
CIRVSDGSTYYADSVKG

SEQ ID NO: 111 - protein, 22 aa, synthetic construct
GSLYTCVQSIVWPARPYYDMDY

SEQ ID NO: 112 - protein, 131 aa, synthetic construct
EVQLLESGGGLVQPGGSLRLSCAASGFTFDDYAIGWFRQAPGKEREGVSCIRVSDGSTYYADSVKGRFTISRDNSKNTLYLQMNSLRAEDTAVYYCAAGSLYTCVQSIVWPARPYYDMDYWGQGTLVTVSS

SEQ ID NO: 113 - protein, 5 aa, synthetic construct
GGGGS

SEQ ID NO: 114 - protein, 6 aa, synthetic construct
SGGGGS

SEQ ID NO: 115 - protein, 10 aa, synthetic construct
GGGGSGGGGS

SEQ ID NO: 116 - protein, 6 aa, synthetic construct
TDYAIG

SEQ ID NO: 117 - protein, 6 aa, synthetic construct
SDYAIG

SEQ ID NO: 118 - protein, 131 aa, synthetic construct
EVQLLESGGGLVQPGGSLRLSCAASGFTFTDYAIGWFRQAPGKEREGVSCIRVSDGSTYYADSVKGRFTISRDSSKNTLYLQMNSLRAEDTAVYYCAAGSLYTCVQSIVWPARPYYDMDYWGQGTLVTVSS

SEQ ID NO: 119 - protein, 131 aa, synthetic construct
EVQLLESGGGLVQPGGSLRLSCAASGFTFTDYAIGWFRQAPGKEREGVSCIRVSDGSTYYADSVKGRFTISRDNAKNTLYLQMNSLRAEDTAVYYCAAGSLYTCVQSIVWPARPYYDMDYWGQGTLVTVSS

SEQ ID NO: 120 - protein, 131 aa, synthetic construct
EVQLLESGGGLVQPGGSLRLSCAASGFTFSDYAIGWFRQAPGKEREGVSCIRVSDGSTYYADSVKGRFTISRDTSKNTLYLQMNSLRAEDTAVYYCAAGSLYTCVQSIVWPARPYYDMDYWGQGTLVTVSS

(19) United States
(12) Patent Application Publication
(10) Pub. No.: US 2025/0019438 A1
(43) Pub. Date: Jan. 16, 2025

(54) Optimized CD3 Antigen Binding Domains

(71) Applicant: MedImmune, LLC, Gaithersburg, MD (US)

(72) Inventors: Yariv MAZOR, Gaithersburg, MD (US); Zachary BRITTON, Gaithersburg, MD (US); Xiuling LI, Gaithersburg, MD (US); Sterling PAYNE, Gaithersburg, MD (US); Even WALSENG, Gaithersburg, MD (US); Chunning YANG, Gaithersburg, MD (US)

(21) Appl. No.: 18/630,379

(22) Filed: Apr. 9, 2024

Related U.S. Application Data
(60) Provisional application No. 63/495,127, filed on Apr. 10, 2023.

(51) Int. Cl. C07K 16/28; A61P 35/00

(52) U.S. Cl. CPC C07K 16/2809; A61P 35/00; C07K 16/2815; C07K 2317/31; C07K 2317/526; C07K 2317/565; C07K 2317/92

(57) ABSTRACT

The present disclosure relates to antibodies or fragments thereof comprising an antigen binding domain capable of binding to a CD3 protein or a fragment thereof. The present disclosure also relates to such antibodies that bind to CD3 having optimized affinity to induce T cell activation, but without being associated with excessive cytokine release and reduced tolerability. The disclosure also relates to methods of producing these antibodies and their therapeutic uses.

### CROSS-REFERENCE TO RELATED APPLICATIONS

This application claims the benefit of U.S. Provisional Patent Application No. 63/495,127, filed Apr. 10, 2023, which is incorporated herein by reference in its entirety.

### REFERENCE TO SEQUENCE LISTING SUBMITTED ELECTRONICALLY

The content of the electronically submitted sequence listing (Name: IOTS-102-US-NP Sequence Listing.xml; Size: 109,285 bytes; and Date of Creation: Apr. 2, 2024), filed with the application, is incorporated herein by reference in its entirety.

### FIELD OF THE DISCLOSURE

The present disclosure relates to antibodies or fragments thereof comprising an antigen binding domain capable of binding to a CD3 protein or a fragment thereof. The disclosure also relates to methods of producing these antibodies and their therapeutic uses.

### BACKGROUND

T cells recognize antigenic peptides via a complex of heterodimeric T cell receptor (TCR) α and β chains, in combination with four CD3 subunits, denoted ε, γ, δ, ζ (Kindt et al., 2007). After TCR-mediated antigen recognition, CD3 is essential for transmitting TCR-trigged signaling through immunoreceptor tyrosine activation motifs (ITAMs) (Letourneur et al., 1992). There remains a need for further antibodies, in particular multispecific antibodies, that are capable of binding to CD3 and transmitting TCR-triggered signaling in a safe and effective manner.

### SUMMARY OF THE DISCLOSURE

Accordingly, there is a need in the art for antibodies that bind to CD3 having optimized affinity to induce T cell activation, but without being associated with excessive cytokine release and reduced tolerability. The present disclosure involved an extensive selection and affinity maturation program to isolate a panel of CD3 antibodies that are capable of binding CD3 and inducing T cell activation. Such molecules are expected to be capable of achieving therapeutic benefit by ensuring effective T cell engagement, but without the safety concerns attributed to antibodies that bind CD3 with a high affinity or cytokine release profile, making them suitable for the treatment of cancer as immunotherapeutic agents.

Accordingly, in one aspect provided herein is an antibody comprising an antigen binding domain that is capable of binding to a CD3 protein or a fragment thereof, wherein the CD3 antigen binding domain comprises a heavy chain variable (VH) region according to any one of the following:
a VH region comprising the following CDRs:
   HCDR1 having the amino acid sequence of SEQ ID NO: 90;
   HCDR2 having the amino acid sequence of SEQ ID NO: 91; and
   HCDR3 having the amino acid sequence of SEQ ID NO: 92,
a VH region comprising the following CDRs:
   HCDR1 having the amino acid sequence of SEQ ID NO: 82;
   HCDR2 having the amino acid sequence of SEQ ID NO: 83; and
   HCDR3 having the amino acid sequence of SEQ ID NO: 84,
a VH region comprising the following CDRs:
   HCDR1 having the amino acid sequence of SEQ ID NO: 78;
   HCDR2 having the amino acid sequence of SEQ ID NO: 79; and
   HCDR3 having the amino acid sequence of SEQ ID NO: 80,
a VH region comprising the following CDRs:
   HCDR1 having the amino acid sequence of SEQ ID NO: 66;
   HCDR2 having the amino acid sequence of SEQ ID NO: 67; and
   HCDR3 having the amino acid sequence of SEQ ID NO: 68,
a VH region comprising the following CDRs:
   HCDR1 having the amino acid sequence of SEQ ID NO: 70;
   HCDR2 having the amino acid sequence of SEQ ID NO: 71; and
   HCDR3 having the amino acid sequence of SEQ ID NO: 72,
a VH region comprising the following CDRs:
   HCDR1 having the amino acid sequence of SEQ ID NO: 74;
   HCDR2 having the amino acid sequence of SEQ ID NO: 75; and
   HCDR3 having the amino acid sequence of SEQ ID NO: 76,
a VH region comprising the following CDRs:
   HCDR1 having the amino acid sequence of SEQ ID NO: 86;
   HCDR2 having the amino acid sequence of SEQ ID NO: 87; and
   HCDR3 having the amino acid sequence of SEQ ID NO: 88,
a VH region comprising the following CDRs:
   HCDR1 having the amino acid sequence of SEQ ID NO: 94;
   HCDR2 having the amino acid sequence of SEQ ID NO: 95; and
   HCDR3 having the amino acid sequence of SEQ ID NO: 96, and
a VH region comprising the following CDRs:
   HCDR1 having the amino acid sequence of SEQ ID NO: 98;
   HCDR2 having the amino acid sequence of SEQ ID NO: 99; and
   HCDR3 having the amino acid sequence of SEQ ID NO: 100,
      and wherein the CD3 antigen binding domain comprises a light chain variable (VL) region according to any one of the following:
a VL region comprising the following CDRs:
   HCDR1 having the amino acid sequence of SEQ ID NO: 46;
   HCDR2 having the amino acid sequence of SEQ ID NO: 47; and
   HCDR3 having the amino acid sequence of SEQ ID NO: 48,
a VL region comprising the following CDRs:
   HCDR1 having the amino acid sequence of SEQ ID NO: 58;
   HCDR2 having the amino acid sequence of SEQ ID NO: 59; and
   HCDR3 having the amino acid sequence of SEQ ID NO: 60, and
a VL region comprising the following CDRs:
   HCDR1 having the amino acid sequence of SEQ ID NO: 62;
   HCDR2 having the amino acid sequence of SEQ ID NO: 63; and
   HCDR3 having the amino acid sequence of SEQ ID NO: 64.

In some cases, the CD3 antigen binding domain comprises a VH region and VL region comprising one of the following sets of CDRs:
i. HCDR1 having the amino acid sequence of SEQ ID NO: 90,
ii. HDCR2 having the amino acid sequence of SEQ ID NO: 91,
iii. HCDR3 having the amino acid sequence of SEQ ID NO: 92,
iv. LCDR1 having the amino acid sequence of SEQ ID NO: 46,
v. LCDR2 having the amino acid sequence of SEQ ID NO: 47
vi. LCDR3 having the amino acid sequence of SEQ ID NO: 48; or
i. HCDR1 having the amino acid sequence of SEQ ID NO: 82,
ii. HDCR2 having the amino acid sequence of SEQ ID NO: 83,
iii. HCDR3 having the amino acid sequence of SEQ ID NO: 84,
iv. LCDR1 having the amino acid sequence of SEQ ID NO: 46,
v. LCDR2 having the amino acid sequence of SEQ ID NO: 47,
vi. LCDR3 having the amino acid sequence of SEQ ID NO: 48; or
i. HCDR1 having the amino acid sequence of SEQ ID NO: 78
ii. HDCR2 having the amino acid sequence of SEQ ID NO: 79
iii. HCDR3 having the amino acid sequence of SEQ ID NO: 80,
iv. LCDR1 having the amino acid sequence of SEQ ID NO: 62,
v. LCDR2 having the amino acid sequence of SEQ ID NO: 63,
vi. LCDR3 having the amino acid sequence of SEQ ID NO: 64,

In some cases, the VH domain of the CD3 antigen binding domain comprises an amino acid sequence having at least 90%, 91%, 92%, 93%, 94%, 95%, 96%, 97%, 98%, 99%, or 100% sequence identity to SEQ ID NO: 77, SEQ ID NO: 89, or SEQ ID NO: 81. In some cases, the VL domain of CD3 antigen binding domain comprises an amino acid sequence having at least 90%, 91%, 92%, 93%, 94%, 95%, 96%, 97%, 98%, 99%, or 100% sequence identity to SEQ ID NO: 45, or SEQ ID NO: 61.

In some cases, the CD3 antigen binding domain comprises:
i. a VH region having at least 90%, 91%, 92%, 93%, 94%, 95%, 96%, 97%, 98%, 99%, or 100% sequence identity to SEQ ID NO: 77, and a VL region having at least 90%, 91%, 92%, 93%, 94%, 95%, 96%, 97%, 98%, 99%, or 100% sequence identity to SEQ ID NO: 61;
ii. a VH region having at least 90%, 91%, 92%, 93%, 94%, 95%, 96%, 97%, 98%, 99%, or 100% sequence identity to SEQ ID NO: 89, and a VL region having at least 90%, 91%, 92%, 93%, 94%, 95%, 96%, 97%, 98%, 99%, or 100% sequence identity to SEQ ID NO: 45 or
iii. a VH region having at least 90%, 91%, 92%, 93%, 94%, 95%, 96%, 97%, 98%, 99%, or 100% sequence identity to SEQ ID NO: 81, and a VL region having at least 90%, 91%, 92%, 93%, 94%, 95%, 96%, 97%, 98%, 99%, or 100% sequence identity to SEQ ID NO: 45.

As described above, CD3 antigen binding domains find particular utility when used in the context of a multispecific antibody that contains an antigen binding domain that is capable of binding to another target. For example the CD3 antigen binding domains described herein may form part of a multispecific antibody that is capable of binding to CD3 and another target, e.g. a tumor associated antigen (TAA). Examples of suitable TAAs are described herein.

Accordingly, in some cases the antibody further comprising a target antigen binding domain. In some cases, the target antigen binding domain is capable of binding to a tumor associated antigen (TAA). In some cases, the antibody is in a ‘2+1’ format, where the antibody contains a single CD3 antigen binding domain that binds CD3 monovalently and two target antigen binding domains that bind the target (e.g. TAA) bivalently. In some cases, the antibody is in a trispecific, trivalent format, containing three antigen binding domains.

When it comes to generating multispecific antibodies containing multiple antigen binding arms formed from different light and heavy chains, promiscuous pairing of heavy and light chains can present challenges. As described herein, amino acid residues at the interface between a lambda light chain and heavy chain where charge pairs could be introduced were identified, demonstrating that the introduction of these lambda charge pairs could advantageously improve chain pairing beyond what was achieved in a previous antibody format. As further described herein, these charge variants can be used to efficiently produce multispecific antibodies in different formats, including in a ‘2+1’ bispecific format and a trivalent, trispecific format.

Accordingly, an antigen binding domain in the antibody (e.g. the CD3 antigen binding arm) contains a lambda charge pair between the constant light chain lambda region (CLλ) and heavy chain constant region 1 (CH1) of that antigen binding arm, wherein the antigen binding domain comprises a lambda charge pair located at one or more of the following pairs of positions:
(i) position 117 in the CLλ and position 141 in the CH1;
(ii) position 117 in the CLλ and position 185 in the CH1;
(iii) position 119 in the CLλ and position 128 in the CH1;
(iv) position 134 in the CLλ and position 128 in the CH1;
(v) position 134 in the CLλ and position 145 in the CH1;
(vi) position 134 in the CLλ and position 183 in the CH1;
(vii) position 136 in the CLλ and position 185 in the CH1;
(viii) position 178 in the CLλ and position 173 in the CH1; and
(ix) position 117 in the CLλ and position 187 in the CH1,
wherein the lambda charge pair comprises a positively charged amino acid residue optionally selected from arginine, lysine or histidine located at one of the positions in the lambda charge pair and a negatively charged amino acid residue optionally selected from aspartic acid, glutamic acid, serine or threonine located at the other position in the lambda charge pair, and
wherein the numbering is according to the EU index.

In some cases, an antigen binding arm in the antibody (e.g. the CD3 antigen binding arm) contains a lambda charge pair located at position 117 in the CLλ and position 141 in the CH1, wherein the lambda charge pair comprises a positively charged amino acid residue selected from arginine, lysine or histidine located at one of the positions in the lambda charge pair and a negatively charged amino acid residue selected from aspartic acid, glutamic acid, serine or threonine located at the other position in the lambda charge pair.

As further described herein, the lambda charge pairs can be combined with other approaches for encouraging light chain pairing, for example in order to further increase the correct assembly of the desired multispecific antibody.

In some instances, the antibody comprises a CD3 antigen binding domain and a CD8 antigen binding domain. For example, the antibody may comprise a CD3 antigen binding domain, a TAA antigen binding domain and a CD8 antigen binding domain. The CD8 antigen binding domain may be a VHH molecule (e.g. fused to the 2+1 bispecific format described herein). The CD8 antigen binding domain may be an antigen binding (‘Fab’) arm (e.g. one of the antigen binding arms in the trispecific antibody format described herein).

Also provided are pharmaceutical compositions comprising the antibody described herein, as further defined herein. Also provided are antibody molecules and pharmaceutical compositions, all as defined herein, for use in a method of treatment of the human or animal body, such as a method of treatment of a cancer. Also provided are methods of treating cancer comprising administering an antibody or pharmaceutical composition as defined herein.

Also provided are nucleic acids, vectors and host cells as defined herein. Also provided is a method of producing an antibody as defined herein. The disclosure includes the combination of the aspects and features described except where such a combination is clearly impermissible or expressly avoided.

### SUMMARY OF THE FIGURES

Instances and experiments illustrating the principles of the disclosure will now be discussed with reference to the accompanying figures in which:

FIGS. 1A, 1B, 1C, and 1D show parental AZ Vκ+AZ VH IgG1-TM, (FIG. 1A) shows binding of the parental AZ Vκ+AZ IgG1-TM antibody to HPB-ALL cells, which express human CD3 (hCD3⁺). (FIG. 1B) shows binding of the parental anti-CD3 AZ Vκ+AZ IgG1-TM antibody to HSC-F cells, which express cynomolgus monkey CD3 (cyCD3+). (FIG. 1C) shows binding of the parental anti-CD3 antibody AZ Vκ+AZ IgG1-TM antibody to Jurkat hCD3+ cells. (FIG. 1D) shows binding of the parental anti-CD3 antibody AZ Vκ+AZ IgG1-TM antibody to T cell receptor (TCR) knock out (KO) Jurkat cells.

FIGS. 2A, 2B, 2C, and 2D show-the binding of variants resulting from mitigation of potential sequence liabilities within CDR L1-2 and CDR H1-2 to CD3+ cells measured by flow cytometry. (FIG. 2A) shows binding of the parental anti-CD3 antibody and variants to HPB-ALL cells, which express human CD3 (hCD3⁺). (FIG. 2B) shows binding of the parental anti-CD3 antibody and AZ variants to HSC-F cells, which express cynomolgus monkey CD3 (cyCD3+). (FIG. 2C) shows binding of the parental anti-CD3 antibody and variants to Jurkat hCD3+ cells. (FIG. 2D) shows binding of the parental anti-CD3 antibody and AZ variants to T cell receptor (TCR) knock out (KO) Jurkat cells.

FIGS. 3A and 3B show-the results of the profiling of anti-CD3 variants for CD3+ cellular binding. Binding to (FIG. 3A) HPB-ALL (hCD3+) and (FIG. 3B) HSC-F (cyCD3+) cells was measured by flow cytometry.

FIG. 4 shows the results of the profiling of anti-CD3 variants for EGFR+ cellular toxicity. The cytotoxicity profiles of EGFR-CD3 variant antibodies were assessed by flow cytometry following 24-hour exposure of T cells with EGFR⁺ NCI H358. Data representing the parental anti-CD3 variant appears in filled black circles. Data representing AZ Vκ 17, 26, and 29-based variants are shown in dark, medium and light gray, respectively.

FIG. 5 shows the results of the profiling of anti-CD3 variants for CD4+ T cell activation. The CD4⁺ T cell activation profiles of EGFR-CD3 variant antibodies were assessed by flow cytometry following 24-hour exposure of T cells with EGFR⁺ NCI H358. Data representing the parental anti-CD3 variant appears in filled black circles. Data representing AZ Vκ 17, 26, and 29-based variants are shown in dark, medium and light gray, respectively.

FIG. 6 shows the results of the profiling of anti-CD3 variants for CD8+ T cell activation. The CD8⁺ T cell activation profiles of EGFR-CD3 variant antibodies were assessed by flow cytometry following 24-hour exposure of T cells with EGFR⁺ NCI H358. Data representing the parental anti-CD3 variant appears in filled black circles. Data representing AZ Vκ 17, 26, and 29-based variants are shown in dark, medium and light gray, respectively.

FIG. 7 shows the results of the profiling of anti-CD3 variants for IL-6 release. The IL-6 cytokine release profiles of EGFR-CD3 variant antibodies were assessed using the ProcartaPlex multiplex immunoassay following 24-hour exposure of T cells with EGFR⁺ NCI H358. Data representing the parental anti-CD3 variant appears in filled black circles. Data representing AZ Vκ 17, 26, and 29-based variants are shown in dark, medium and light gray, respectively.

FIG. 8 shows the results of the profiling of anti-CD3 variants for TNF-α release. The TNF-α cytokine release profiles of EGFR-CD3 variant antibodies were assessed using the ProcartaPlex multiplex immunoassay following 24-hour exposure of T cells with EGFR⁺ NCI H358. Data representing the parental anti-CD3 variant appears in filled black circles. Data representing AZ Vκ 17, 26, and 29-based variants are shown in dark, medium and light gray, respectively.

FIG. 9 shows the results of the profiling of anti-CD3 variants for IFN-γ release. The IFN-γ cytokine release profiles of EGFR-CD3 variant antibodies were assessed using the ProcartaPlex multiplex immunoassay following 24-hour exposure of T cells with EGFR⁺ NCI H358. Data representing the parental anti-CD3 variant appears in filled black circles. Data representing AZ Vκ 17, 26, and 29-based variants are shown in dark, medium and light gray, respectively.

FIG. 10 shows the results of the profiling of anti-CD3 variants for IL-2 release. The IL-2 cytokine release profiles of EGFR-CD3 variant antibodies were assessed using the ProcartaPlex multiplex immunoassay following 24-hour exposure of T cells with EGFR⁺ NCI H358. Data representing the parental anti-CD3 variant appears in filled black circles. Data representing AZ Vκ 17, 26, and 29-based variants are shown in dark, medium and light gray, respectively.

FIG. 11 shows the results of the profiling of anti-CD3 variants for IL-10 release. The IL-10 cytokine release profiles of EGFR-CD3 variant antibodies were assessed using the ProcartaPlex multiplex immunoassay following 24-hour exposure of T cells with EGFR⁺ NCI H358. Data representing the parental anti-CD3 variant appears in filled black circles. Data representing AZ Vκ 17, 26, and 29-based variants are shown in dark, medium and light gray, respectively.

FIG. 12 shows the results of the profiling of anti-CD3 variants for FasL release. The FasL cytokine release profiles of EGFR-CD3 variant antibodies were assessed using the ProcartaPlex multiplex immunoassay following 24 hr exposure of T cells with EGFR⁺ NCI H358. Data representing the parental anti-CD3 variant appears in filled black circles. Data representing AZ Vκ 17, 26, and 29-based variants are shown in dark, medium and light gray, respectively.

FIG. 13 shows the results of the profiling of anti-CD3 variants for Granzyme A release. The Granzyme A cytokine release profiles of EGFR-CD3 variant antibodies were assessed using the ProcartaPlex multiplex immunoassay following 24-hour exposure of T cells with EGFR⁺ NCI H358. Data representing the parental anti-CD3 variant appears in filled black circles. Data representing AZ Vκ 17, 26, and 29-based variants are shown in dark, medium and light gray, respectively.

FIG. 14 shows the results of the profiling of anti-CD3 variants for Granzyme B release. The Granzyme B cytokine release profiles of EGFR-CD3 variant antibodies were assessed using the ProcartaPlex multiplex immunoassay following 24-hour exposure of T cells with EGFR⁺ NCI H358. Data representing the parental anti-CD3 variant appears in filled black circles. Data representing AZ Vκ 17, 26, and 29-based variants are shown in dark, medium and light gray, respectively.

FIG. 15 shows the results of the profiling of anti-CD3 variants for IL-17A release. The IL-17A cytokine release profiles of EGFR-CD3 variant antibodies were assessed using the ProcartaPlex multiplex immunoassay following 24-hour exposure of T cells with EGFR⁺ NCI H358. Data representing the parental anti-CD3 variant appears in filled black circles. Data representing AZ Vκ 17, 26, and 29-based variants are shown in dark, medium and light gray, respectively.

FIG. 16 shows the results of the profiling of anti-CD3 variants for Perforin release. The Perforin cytokine release profiles of EGFR-CD3 variant antibodies were assessed using the ProcartaPlex multiplex immunoassay following 24-hour exposure of T cells with EGFR⁺ NCI H358. Data representing the parental anti-CD3 variant appears in filled black circles. Data representing AZ Vκ 17, 26, and 29-based variants are shown in dark, medium and light gray, respectively.

FIGS. 17A and 17B show EGFR⁺ dependent cytotoxicity and T cell activation for selected anti-CD3 variants. The cytotoxicity (FIG. 17A), CD4⁺ T cell activation (FIG. 17B) and CD8⁺ T cell activation (FIG. 17C) profiles of select EGFR-CD3 variant antibodies were assessed by flow cytometry following 24-hour exposure of T cells with EGFR⁺ MDA-MB-468. Results show EGFR-dependent responses.

FIGS. 18A and 18B show that anti-CD3 variants exhibit reduced T cell activation compared to the parental anti-CD3 in the absence of EGFR+ cells. The CD4⁺ (FIG. 18A) and CD8⁺ (FIG. 18B) T cell activation profiles were assessed by flow cytometry after 48-hour exposure to immobilized EGFR-CD3 variant antibodies. Data representing the parental anti-CD3 variant appears in filled black bars. Data representing AZ Vκ 17, 26, and 29-based variants are shown in dark, medium and light gray bars, respectively.

FIG. 19 shows the kinetics measurements of anti-CD3 variants. Kinetics measurement to the soluble form of CD3 were obtained using an Octet384 instrument. The dissociation constants, KD, were calculated as a ratio of k_{off}/kₒₙ from a non-linear fit of the data.

FIG. 20 contains a schematic of the DuetMab antibodies containing charge pairs. The left hand “hole” HC is disulfide bonded via native cysteines to a kappa LC and contains a kappa charge pair (e.g. S183K/V133E), indicated by the minus (“−”) symbol on the kappa LC and the plus (“+”) symbol on the “hole” HC. The right hand “knob” HC is disulfide bonded via engineered cysteines to the lambda LC and contains a lambda charge pair, indicated by the plus (“+”) symbol on the lambda LC and the minus (“−”) symbol on the “knob” HC.

FIG. 21 contains a schematic of the DuetMab ‘2+1’ antibodies containing charge pairs. The right hand “knob” HC is disulfide bonded via engineered cysteines to the lambda LC and contains a lambda charge pair, indicated by the plus (“+”) symbol on the lambda LC and the minus (“−”) symbol on the “knob” HC. The CH1 and VH region of this knob HC and lambda LC form a “CD3 antigen binding arm”. The left hand “hole” HC is disulfide bonded via native cysteines to a kappa LC and contains a kappa charge pair (e.g. S183K/V133E), indicated by the minus (“−”) symbol on the kappa LC and the plus (“+”) symbol on the “hole” HC. A third antigen binding arm is fused from the N-terminus of its CH1 to the C-terminus of the “knob” HC by a peptide linker. The CH1 of the third antigen binding arm is disulfide bonded via native cysteines to a kappa LC and contains a kappa charge pair. As indicated by the different shading, the first antigen binding arm binds a first epitope (CD3) and the second and third binding arms bind a second, different epitope.

FIG. 22 contains a schematic of the ‘TriMab’ trispecific antibodies containing charge pairs. The right hand “knob” HC is disulfide bonded via engineered cysteines to the lambda LC and contains a lambda charge pair, indicated by the plus (“+”) symbol on the lambda LC and the minus (“−”) symbol on the “knob” HC. The CH1 and VH region of this knob HC and lambda LC form a “first antigen binding arm”. The left hand “hole” HC is disulfide bonded via native cysteines to a kappa LC and contains a kappa charge pair, indicated by the minus (“−”) symbol on the kappa LC and the plus (“+”) symbol on the “hole” HC. The CH1 and VH region of this “hole” HC and kappa LC form a “second antigen binding arm”. A third antigen binding arm is fused from the N-terminus of its CH1 to the C-terminus of the “knob” HC by a peptide linker. The CH1 of the third antigen binding arm is disulfide bonded via engineered cysteines to a kappa LC and contains a kappa charge pair, with the opposite charges to the second antigen binding arm—a negatively charged amino acid residue (“−”) in CH1 and a positively charged amino acid residue (“+”) in the kappa LC. As indicated by the different shading, the first antigen binding arm (dark shading) binds a first epitope, the second antigen binding arm (light shading) binds a second epitope, and the third binding arm (hatched shading) binds a third epitope.

### DETAILED DESCRIPTION OF THE DISCLOSURE

Aspects and instances the present disclosure will now be discussed with reference to the accompanying figures. Further aspects and instances will be apparent to those skilled in the art. All documents mentioned in this text are incorporated herein by reference.

### Antibodies

The present disclosure relates to antibodies. Antibodies according to the present disclosure may be provided in isolated form, in the sense of being free from contaminants, such as antibodies able to bind other polypeptides and/or serum components.

The term “antibody” or “antibody molecule” describes an immunoglobulin whether natural or partly or wholly synthetically produced. The antibody may be human or humanized. In some aspects, the antibody is a monoclonal antibody. Examples of antibodies are the immunoglobulin isotypes, such as immunoglobulin G (IgG), and their isotypic subclasses, such as IgG1, IgG2, IgG3 and IgG4, as well as fragments thereof.

The term “antibody”, as used herein, includes multispecific antibodies and antibody fragments. A multispecific antibody is an antibody that comprises at least two antigen-binding domains, each of which being capable of binding to a different target. “Antibody fragments” refers to a portion of the full-length antibody as long as they display binding to the relevant target molecule(s). Typically an antibody fragment will contain the antigen binding or variable region thereof.

An antibody is typically composed of two different types of polypeptide chains: one termed a heavy chain and the other terms a light chain. A natural monospecific antibody consists of two identical heavy chains and two identical light chains. The two heavy chains are typically linked to each other by disulfide bonds and each heavy chain is typically linked to a light chain by a disulfide bond. The disulfide bonds linking the light and heavy chains are sometimes termed “inter-chain” disulfide bonds, to distinguish them from the “intra-chain” disulfide bonds that are present within the individual heavy and light chain polypeptides.

The formation of disulfide bonds between cysteine residues occurs during the folding of many proteins that enter the secretory pathway. As the polypeptide chain collapses, cysteines brought into proximity can form covalent linkages during a process catalyzed by members of the protein disulfide isomerase family.

The term “disulfide link” or “disulfide linked” as used herein, refers to the single covalent bond formed from the coupling of thiol groups, especially of cysteine residues. In some aspects, the covalent linkage between two cysteines is between the two sulfur atoms of each residue. However, depending on the environment, not all protein species may have a disulfide present at all times, for example, in the event of disulfide reduction. Thus, the term “disulfide link” or “disulfide linked” (whether native or engineered), in some aspects, also refers to the presence of two cysteine residues that are capable of forming a disulfide link, irrespective of whether or not they are actually linked at that individual point in time.

All light chains in natural antibodies are either “lambda λ” or “kappa κ” light chains, which differ in terms of their amino acid sequence. Light chains are composed of a single constant light chain region (CL) and a single light chain variable region (VL). An example of a constant light chain lambda region (CLλ) amino acid sequence is provided as SEQ ID NO: 105 and an example of a constant light chain kappa region (CLκ) amino acid sequence is provided as SEQ ID NO: 106. Light chains used in the antibodies described herein may be chimeric light chains, e.g. contain a CLλ and a VLκ.

IgG heavy chains are composed of a heavy variable (VH) region and three heavy constant regions (CH1, CH2 and CH3), with an additional “hinge region” between CH1 and CH2. An example of an IgG1 CH1 region amino acid sequence is provided as SEQ ID NO: 101. An example of an IgG1 CH2 amino acid sequence is provided as SEQ ID NO: 102. An example of an IgG1 CH3 amino acid sequence is provided as SEQ ID NO: 103. An example of an heavy chain amino acid sequence comprising a CH1, hinge, CH2 and CH3 is provided as SEQ ID NO: 104.

Unless otherwise specified, amino acid residue positions in the constant domain, including the position of amino acid sequences, substitutions, deletions and insertions as described herein, are numbered according to EU numbering (Edelman, 2007).

The light chain associates with the VH and CH1 to form an “antigen binding arm”, also referred to herein as “target binding arm” and the variable domains in the antigen binding arm interact to form the “antigen binding domain”.

An “antigen binding domain” describes the part of a molecule that binds to all or part of the target antigen and generally comprises six complementarity-determining regions (CDRs); three in the VH region: HCDR1, HCDR2 and HCDR3, and three in the VL region: LCDR1, LCDR2, and LCDR3. The six CDRs together define the paratope of the antigen binding domain, which is the part of the antigen binding domain which binds to the target antigen.

The VH region and VL region comprise framework regions (FRs) either side of each CDR, which provide a scaffold for the CDRs. From N-terminus to C-terminus, VH regions comprise the following structure: N term-[HFR1]-[HCDR1]-[HFR2]-[HCDR2]-[HFR3]-[HCDR3]-[HFR4]-C term; and VL regions comprise the following structure: N term-[LFR1]-[LCDR1]-[LFR2]-[LCDR2]-[LFR3]-[LCDR3]-[LFR4]-C term. Typically, the antigen binding domain comprises the VH and VL region.

An “antigen binding arm” as described herein comprises the antigen binding domain (e.g. the VH and VL region) and also a CH1 and a constant light chain (i.e. at least one constant and one variable domain of each of the heavy and light chain), where the constant light chain is disulfide linked to the CH1. A monoclonal monospecific IgG antibody molecule contains two antigen binding arms, each of which are able to bind the same target (i.e. it is bivalent for a single target).

Examples of antibody fragments include Fab, Fab′, F(ab′)2, Fd and Fv fragments. Diabodies; linear antibodies; single-chain antibody molecules; and multispecific antibodies are antibodies formed from these antibody fragments.

Traditionally, these fragments were derived via a proteolytic digestion of intact antibodies using techniques known in the art. However, these fragments can now be produced directly by recombinant host cells. Fab, Fv and scFv antibody fragments can all be expressed in and secreted from E. coli, thus allowing the facile production of large amounts of these fragments. In one aspect, the antibody fragments can be isolated from the antibody phage libraries discussed elsewhere herein. Fab′-SH fragments can also be directly recovered from E. coli and chemically coupled to form F(ab′)2 fragments. F(ab′)2 fragments can also be isolated directly from recombinant host cell culture. Other techniques for the production of antibody fragments are known in the art. In certain aspects, antibodies provided herein comprise a single-chain Fv fragment (scFv) or other antigen binding domain.

In some instances, the antibody fragments described herein comprise domain antibodies, e.g. antibodies containing the small functional binding units of antibodies corresponding to the carriable region of the VH and VL chains of human antibodies.

### CD3 Antigen Binding Domain

The present disclosure provides antibodies comprising an antigen binding domain that is capable of binding to a CD3 protein or a fragment thereof. Such antigen binding domains are also referred to herein as “CD3 antigen binding domains”. In some cases, the antibodies comprising the antigen binding domains bind CD3 monovalently (e.g. the antibody only contains a single CD3 antigen binding domain).

CD3 (cluster of differentiation 3) is a protein complex composed of four subunits, the CD3γ chain, the CD3δ chain, and two CD3ε chains. CD3 associates with the T-cell receptor and the ζ chain to generate an activation signal in T lymphocytes.

The CD3 antigen binding domain comprises the CDRs of an antibody which is capable of binding to CD3. In some cases, the CD3 antigen binding domain comprises a VH region and/or a VL region which is, or has at least 70% identity to, the VH and/or VL region of an antibody that binds CD3 as exemplified herein.

In some cases, the CD3 antigen binding domain comprises a VH region according to any one of (1) to (9) below:

An antibody comprising an antigen binding domain that is capable of binding to a CD3 protein or a fragment thereof, wherein the CD3 antigen binding domain comprises a heavy chain variable (VH) region according to any one of the following:
(1) a VH region comprising the following CDRs:
   HCDR1 having the amino acid sequence of SEQ ID NO: 90;
   HCDR2 having the amino acid sequence of SEQ ID NO: 91; and
   HCDR3 having the amino acid sequence of SEQ ID NO: 92,
(2) a VH region comprising the following CDRs:
   HCDR1 having the amino acid sequence of SEQ ID NO: 82;
   HCDR2 having the amino acid sequence of SEQ ID NO: 83; and
   HCDR3 having the amino acid sequence of SEQ ID NO: 84,
(3) a VH region comprising the following CDRs:
   HCDR1 having the amino acid sequence of SEQ ID NO: 78;
   HCDR2 having the amino acid sequence of SEQ ID NO: 79; and
   HCDR3 having the amino acid sequence of SEQ ID NO: 80,
(4) a VH region comprising the following CDRs:
   HCDR1 having the amino acid sequence of SEQ ID NO: 66;
   HCDR2 having the amino acid sequence of SEQ ID NO: 67; and
   HCDR3 having the amino acid sequence of SEQ ID NO: 68,
(5) a VH region comprising the following CDRs:
   HCDR1 having the amino acid sequence of SEQ ID NO: 70;
   HCDR2 having the amino acid sequence of SEQ ID NO: 71; and
   HCDR3 having the amino acid sequence of SEQ ID NO: 72,
(6) a VH region comprising the following CDRs:
   HCDR1 having the amino acid sequence of SEQ ID NO: 74;
   HCDR2 having the amino acid sequence of SEQ ID NO: 75; and
   HCDR3 having the amino acid sequence of SEQ ID NO: 76,
(7) a VH region comprising the following CDRs:
   HCDR1 having the amino acid sequence of SEQ ID NO: 86;
   HCDR2 having the amino acid sequence of SEQ ID NO: 87; and
   HCDR3 having the amino acid sequence of SEQ ID NO: 88,
(8) a VH region comprising the following CDRs:
   HCDR1 having the amino acid sequence of SEQ ID NO: 94;
   HCDR2 having the amino acid sequence of SEQ ID NO: 95; and
   HCDR3 having the amino acid sequence of SEQ ID NO: 96, and
(9) a VH region comprising the following CDRs:
   HCDR1 having the amino acid sequence of SEQ ID NO: 98;
   HCDR2 having the amino acid sequence of SEQ ID NO: 99; and
   HCDR3 having the amino acid sequence of SEQ ID NO: 100,

In some cases, the CD3 antigen binding domain comprises a VL region according to any one of (1) to (3) below:
(10) a VL region comprising the following CDRs:
   HCDR1 having the amino acid sequence of SEQ ID NO: 46;
   HCDR2 having the amino acid sequence of SEQ ID NO: 47; and
   HCDR3 having the amino acid sequence of SEQ ID NO: 48,
(11) a VL region comprising the following CDRs:
   HCDR1 having the amino acid sequence of SEQ ID NO: 58;
   HCDR2 having the amino acid sequence of SEQ ID NO: 59; and
   HCDR3 having the amino acid sequence of SEQ ID NO: 60, and
(12) a VL region comprising the following CDRs:
   HCDR1 having the amino acid sequence of SEQ ID NO: 62;
   HCDR2 having the amino acid sequence of SEQ ID NO: 63; and
   HCDR3 having the amino acid sequence of SEQ ID NO: 64.

In some cases, the CD3 antigen binding domain comprises a VH region according to any one of (1) to (9) and a VL region according to any one of (10) to (12) above. For example, the CD3 antigen binding domain may comprise:
a VH region comprising the CDRs according to (1) and a VL region comprising the CDRs according to (10);
a VH region comprising the CDRs according to (2) and a VL region comprising the CDRs according to (10);
a VH region comprising the CDRs according to (3) and a VL region comprising the CDRs according to (10);
a VH region comprising the CDRs according to (4) and a VL region comprising the CDRs according to (10);
a VH region comprising the CDRs according to (5) and a VL region comprising the CDRs according to (10);
a VH region comprising the CDRs according to (6) and a VL region comprising the CDRs according to (10);
a VH region comprising the CDRs according to (7) and a VL region comprising the CDRs according to (10);
a VH region comprising the CDRs according to (8) and a VL region comprising the CDRs according to (10);
a VH region comprising the CDRs according to (9) and a VL region comprising the CDRs according to (10);
a VH region comprising the CDRs according to (1) and a VL region comprising the CDRs according to (11);
a VH region comprising the CDRs according to (2) and a VL region comprising the CDRs according to (11);
a VH region comprising the CDRs according to (3) and a VL region comprising the CDRs according to (11);
a VH region comprising the CDRs according to (4) and a VL region comprising the CDRs according to (11);
a VH region comprising the CDRs according to (5) and a VL region comprising the CDRs according to (11);
a VH region comprising the CDRs according to (6) and a VL region comprising the CDRs according to (11);
a VH region comprising the CDRs according to (7) and a VL region comprising the CDRs according to (11);
a VH region comprising the CDRs according to (8) and a VL region comprising the CDRs according to (11);
a VH region comprising the CDRs according to (9) and a VL region comprising the CDRs according to (11);
a VH region comprising the CDRs according to (1) and a VL region comprising the CDRs according to (12);
a VH region comprising the CDRs according to (2) and a VL region comprising the CDRs according to (12);
a VH region comprising the CDRs according to (3) and a VL region comprising the CDRs according to (12);
a VH region comprising the CDRs according to (4) and a VL region comprising the CDRs according to (12);
a VH region comprising the CDRs according to (5) and a VL region comprising the CDRs according to (12);
a VH region comprising the CDRs according to (6) and a VL region comprising the CDRs according to (12);
a VH region comprising the CDRs according to (7) and a VL region comprising the CDRs according to (12);
a VH region comprising the CDRs according to (8) and a VL region comprising the CDRs according to (12); or
a VH region comprising the CDRs according to (9) and a VL region comprising the CDRs according to (12).

In some cases, the CD3 antigen binding domain does not comprise a VH region comprising the CDRs according to according to (1) and a VL region comprising the CDRs according to (12).

In some cases, the CD3 antigen binding domain comprises:
(13) a VH region comprising the CDRs according to (3) and a VL region comprising the CDRs according to (12);
(14) a VH region comprising the CDRs according to (1) and a VL region comprising the CDRs according to (10); or
(15) a VH region comprising the CDRs according to (2) and a VL region comprising the CDRs according to (10).

In some cases, the CD3 antigen binding domain comprises a VH region according to any one of (16) to (24) below:
(16) a VH region comprising the CDRs according to (1) and comprising an amino acid sequence having at least 70% sequence identity, or at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 89;
(17) a VH region comprising the CDRs according to (2) and comprising an amino acid sequence having at least 70% sequence identity, or at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 81;
(18) a VH region comprising the CDRs according to (3) and comprising an amino acid sequence having at least 70% sequence identity, or at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 77;
(19) a VH region comprising the CDRs according to (4) and comprising an amino acid sequence having at least 70% sequence identity, or at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 65;
(20) a VH region comprising the CDRs according to (5) and comprising an amino acid sequence having at least 70% sequence identity, or at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 69;
(21) a VH region comprising the CDRs according to (6) and comprising an amino acid sequence having at least 70% sequence identity, or at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 73;
(22) a VH region comprising the CDRs according to (7) and comprising an amino acid sequence having at least 70% sequence identity, or at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 85;
(23) a VH region comprising the CDRs according to (8) and comprising an amino acid sequence having at least 70% sequence identity, or at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 93; and
(24) a VH region comprising the CDRs according to (9) and comprising an amino acid sequence having at least 70% sequence identity, or at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 97.

In some cases, the CD3 antigen binding domain comprises a VL region according to any one of (25) to (27) below:
(25) a VL region comprising the CDRs according to (10) and comprising an amino acid sequence having at least 70% sequence identity, or at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 45;
(26) a VL region comprising the CDRs according to (11) and comprising an amino acid sequence having at least 70% sequence identity, or at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 57; and
(27) a VL region comprising the CDRs according to (12) and comprising an amino acid sequence having at least 70% sequence identity, or at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 61;

In some cases, the CD3 antigen binding domain comprises a VH region according to any one of (16) to (24) and a VL region according to any one of (25) to (27) above. For example, the CD3 antigen binding domain may comprise:
a VH region according to (16) and a VL region according to (25);
a VH region according to (17) and a VL region according to (25);
a VH region according to (18) and a VL region according to (25);
a VH region according to (19) and a VL region according to (25);
a VH region according to (20) and a VL region according to (25);
a VH region according to (21) and a VL region according to (25);
a VH region according to (22) and a VL region according to (25);
a VH region according to (23) and a VL region according to (25);
a VH region according to (24) and a VL region according to (25);
a VH region according to (16) and a VL region according to (26);
a VH region according to (17) and a VL region according to (26);
a VH region according to (18) and a VL region according to (26);
a VH region according to (19) and a VL region according to (26);
a VH region according to (20) and a VL region according to (26);
a VH region according to (21) and a VL region according to (26);
a VH region according to (22) and a VL region according to (26);
a VH region according to (23) and a VL region according to (26);
a VH region according to (24) and a VL region according to (26);
a VH region according to (16) and a VL region according to (27);
a VH region according to (17) and a VL region according to (27);
a VH region according to (18) and a VL region according to (27);
a VH region according to (19) and a VL region according to (27);
a VH region according to (20) and a VL region according to (27);
a VH region according to (21) and a VL region according to (27);
a VH region according to (22) and a VL region according to (27);
a VH region according to (23) and a VL region according to (27); or
a VH region according to (24) and a VL region according to (27).

In some cases, the CD3 antigen binding domain does not comprise a VH region according to (16) and a VL region according to (27).

In some cases, the CD3 antigen binding domain comprises:
(28) a VH region according to (18) and a VL region according to (27);
(29) a VH region according to (16) and a VL region according to (25); or
(30) a VH region according to (17) and a VL region according to (25).

The antibodies described herein may exhibit reduced toxicity due to a lower binding affinity while maintaining T cell activation, thus ensuring T cell engagement. The antibody described herein may be characterized by CD3 antigen binding domain having a particular affinity for CD3. The binding affinity of an antibody molecule to a cognate antigen, such as human CD3 (e.g. recombinant human CD3εδ, such as that available from Acro Biosystems, CDD-H82W6) can be determined by surface plasmon resonance (SPR), using Octet analysis or Biacore, for example. The binding affinity can be determined using an antibody, for example as part of a multispecific antibody molecule that comprises the CD3 antigen binding domain and another antigen binding domain. Alternatively, the binding affinity can be determined using an antibody that is monospecific for CD3.

Binding affinity is typically measured by K_{d} (the equilibrium dissociation constant between the antigen binding domain and its antigen). As is well understood, the lower the K_{d} value, the higher the binding affinity of the antigen binding domain. For example, an antigen binding domain that binds to a target with a K_{d} of 10 nM would be considered to be binding said target with a higher affinity than an antigen binding domain that binds to the same target with a K_{d} of 100 nM.

In some instances, the CD3 antigen binding domain binds to human CD3 with an affinity having a K_{d} equal to or higher than 1×10⁸ M, equal to or higher than 9×10⁷ M, equal to or higher than 8×10⁷ M. In some instances, the CD3 antigen binding domain binds to human CD3 with an affinity having a K_{d} between 1×10⁸ M and 1×10⁶, between 9×10⁷ M and 1×10⁶, or between 8×10⁷ M and 1×10⁶. In some instances, the CD3 antigen binding domain binds to human CD3 with an affinity having a K_{d} between 1×10⁸ M and 2×10⁶, between 9×10⁷ M and 2×10⁶, or between 8×10⁷ M and 2×10⁶. Optionally the K_{d} is measured using surface plasmon resonance, e.g. using Octet analysis as described in the examples.

The CD3 antigen binding domain may bind to human CD3 with an affinity that is similar to that of an antigen binding domain according to (28), (29) or (30) as set out above. For example, the CD3 antigen binding domain may bind to human CD3 with an affinity having a K_{d} that is less than 5-fold different, less than 4-fold different, less than 3-fold different, less than 2-fold different, less than 1-fold different or less than 0.5-fold different than an antigen binding domain according to (28), (29) or (30) as set out above.

The CD3 antigen binding domain may classified as being able to specifically bind CD3. The term “specific” may refer to the situation in which the antigen binding domain will not show any significant binding to molecules other than its specific binding partner(s), here CD3. Such molecules are referred to as “non-target molecules”. The term “specific” is also applicable where the antibody molecule is specific for particular epitopes, such as epitopes on CD3, that are carried by a number of antigens in which case the antibody molecule will be able to bind to the various antigens carrying the epitope.

In some cases, the CD3 antigen binding domain is considered to not show any significant binding to a non-target molecule if the extent of binding to a non-target molecule is less than about 10% of the binding of the antibody to the target as measured, e.g., by ELISA, SPR, Bio-Layer Interferometry (BLI), MicroScale Thermophoresis (MST), or by a radioimmunoassay (RIA). Alternatively, the binding specificity may be reflected in terms of binding affinity, where the CD3 antigen binding domain described herein binds to CD3 with an affinity that is at least 0.1 order of magnitude greater than the affinity towards another, non-target molecule. In some cases, CD3 antigen binding domain described herein binds to CD3 with an affinity that is one of at least 0.2, 0.3, 0.4, 0.5, 0.6, 0.7, 0.8, 0.9, 1.0, 1.5, or 2.0 orders of magnitude greater than the affinity towards another, non-target molecule.

The CD3 antigen binding domain may additionally be classified by its ability to induce T cell activation. Methods of measuring T cell activation are well known in the art. For example, T cell activation may be assessed by measuring the up-regulation of activation markers, such as CD69 and/or CD25 on CD4+ T cells and/or CD8+ T cells using flow cytometry. CD69 is a membrane-bound, type II C-lectin receptor and is an early activation marker of T cell activation due to its rapid appearance on the surface of the plasma membrane after T cell activation (Cibrian et al., 2017). CD25 is component of the IL-2 receptor, important in T cell proliferation, and is a late activation marker is important in T cell proliferation and activation, and up-regulation of CD25 at the cell surface indicates late T cell activation. Alternative ways of measuring T cell activation are envisaged, such as, for example, measuring T cell proliferation, or measuring the production of effector cytokines (e.g. IFN-γ, and TNF-α) (Zappasodi, et al., 2020).

Accordingly, T cell activation can be measured by calculating the percentage of CD4+ T cells and/or CD8+ T cells that are positive for CD69 and/or CD25 using flow cytometry, as was done in the examples. For example, an antigen binding domain that induces CD69 and CD25 upregulation in 20% of CD4+ T cells has decreased T cell activity compared to an antigen binding domain that induces CD69 and CD25 upregulation in 80% of CD4+ T cells, as measured by flow cytometry.

As described herein, multispecific antibodies that bind to a target and CD3 aim to induce T cell activation only upon engagement of both a target (non-T cell) cell and a T cell. Off-target T cell activity can occur when the multispecific antibody induces T cell activation without engaging a target cell. Off-target T cell activation can be measured using a multispecific antibody molecule that comprises the CD3 antigen binding domain and another antigen binding domain that binds a different target antigen (e.g. a tumor associated antigen (TAA) antigen binding domain) in a T cell activation assay where cells expressing the target antigen are absent.

In some instances, the CD3 antigen binding domain described herein exhibits reduced off-target T cell activity as compared to a control antigen binding domain having a VH domain sequence shown as SEQ ID NO: 5 and a VL domain sequence shown as SEQ ID NO: 1. In some cases, the CD3 antigen binding domain described herein exhibits off-target T cell activity that is at least 2-fold, at least 3-fold, at least 4-fold lower as compared to a control antigen binding domain having a VH domain sequence shown as SEQ ID NO: 5 and a VL domain sequence shown as SEQ ID NO: 1. Off-target T cell activation can be determined using the antibody in a T cell activation assay in the absence of engagement with a target cell. In some instances, off-target T cell activation is determined by calculating the percentage of CD4+ T cells and/or CD8+ T cells that are positive for CD69 and/or CD25 using flow cytometry, in the absence of target cells expressing a target antigen that the antibody is capable of binding.

In some instances, the CD3 antigen binding domain described herein exhibits T cell activity as measured by cytokine release by CD4+ and/or CD8+ T cells upon CD3 binding, wherein the T cell activity is reduced as compared with a parental anti-CD3 control antibody. Cytokines may be selected from the group consisting of: IL-6, TNF-α, INF-γ, IL-10, FasL, Granzyme A, Granzyme B, IL-17A, and perforin. Methods of measuring cytokine release are well known in the art and include, for example, measuring the concentrations of cytokines in the supernatants of a T cell culture using an immunoassay, such as an ELISA. Keeping the release of cytokines, in particular pro-inflammatory cytokines, under control following T cell activation is beneficial as it reduces the risk of cytokine release syndrome (CRS).

Hyperactivation of immune cells, such as T cells, may lead to CRS, which is a serious condition characterized by the hypersecretion of pro-inflammatory cytokines. In some instances, the antibody described herein, exhibits reduced cytotoxicity compared with a parental anti-CD3 control antibody.

Assays to measure cytotoxicity can include measuring cell death by flow cytometry using Propidium lodide (Crowley et al., 2016), or dyes that bind covalently to free amines on the surface and inside of cells (Such as CellTrace™).

### Multispecific Antibodies

As noted above, CD3 antigen binding domains are useful when used in a multispecific antibody format. For example, a multispecific antibody may contain a CD3 antigen binding domain as described herein and a target antigen binding domain capable of binding a target that is not CD3. The target antigen binding domain may be capable of binding a tumor-associated antigen (TAA). Such multispecific antibodies can be used for simultaneous binding to the TAA expressed on cancer cells and CD3 on a T cell, thereby forcing a temporary interaction between the target cell and T cell, causing cross-linking, T-cell activation, and subsequent antigen-dependent T cell killing of the target cell.

In some cases, the antibody comprises a CD3 antigen binding domain and target antigen binding domain. In some cases, the antibody further comprises an antigen binding domain that is capable of binding a TAA (also referred to herein as a TAA antigen binding domain). The antibody may be capable of binding CD3 monovalently and the TAA monovalently (e.g. the antibody comprises one CD3 antigen binding domain and one TAA antigen binding domain).

Examples of TAAs include AFP, aₙb3 (vitronectin receptor), aₙb₆, B-cell maturation agent (BCMA), CA125 (MUC16), CD4, CD20, CD22, CD33, CD52, CD56, CD66e, CD80, CD140b, CD227 (MUC1), EGFR (HER1), EpCAM, GD3 ganglioside, HER2, prostate-specific membrane antigen (PSMA), prostate specific antigen (PSA), CD5, CD19, CD21, CD25, CD37, CD30, CD33, CD45, HLA-DR, anti-idiotype, carcinoembryonic antigen (CEA), e.g. carcinoembryonic antigen-related cell adhesion molecule 5 (CEACAM5), TAG-72, Folate-binding protein, A33, G250, ferritin, glycolipids such as gangliosides, carbohydrates such as CA-125, IL-2 receptor, fibroblast activation protein (FAP), IGF1 R, B7H3, B7H4, PD-L1, CD200, EphA2, c-Met, and mesothelin or variants thereof. In some aspects, the TAAs include EGFR, HER2, STEAP2, GPC3, and c-Met.

In some instances, the antibody comprises a CD3 antigen binding domain and a CD8 antigen binding domain. For example, the antibody may comprise a CD3 antigen binding domain, a TAA antigen binding domain and a CD8 antigen binding domain.

CD8 (cluster of differentiation 8) is a dimer consisting of a pair of CD8 chains. The most common form of CD8 is composed of a CD8-α and CD8-β chain. CD8 serves as the coreceptor on MCHI-restricted T-cells and acts to enhance the antigen sensitivity of CD8⁺ T-cells by binding to a largely invariant region of MHCI at a site distinct from where the T-cell receptor binds. Without wishing to be bound by theory, including an antigen binding domain that is capable of binding CD8 in the multispecific antibody is believed to allow for preferential activation of CD8⁺ T-cells, which may provide superior therapeutic efficacy.

In some instances, the multispecific antibody is in a ‘2+1’ format. In the ‘2+1 format’ the antibody contains an antigen binding domain that binds a first epitope monovalently, and two further antigen binding domains that bind to a second epitope bivalently, where the second epitope is different to the first epitope. The 2+1 format of multispecific antibodies is well-suited for CD3 binding as the goal is to bond only monovalently to the CD3 protein such that the T-cell receptor is only cross-linked and activated upon binding of the target cell. Accordingly, in some instances, the antibody comprises the CD3 antigen binding domain described herein, and two target antigen binding domains that are capable of binding the same target (e.g. a TAA).

In the ‘2+1’ format, one of the two target antigen binding domains is fused to any of the other antigen binding domains present in the antibody, typically via a peptide linker. Suitable peptide linkers are known in the art and may consist of 5 to 100 amino acids, 5 to 50 amino acids, 5 to 25 amino acids, or 5 to 15 amino acids. Peptide linkers are formed mainly from glycine and serine amino acid residues and can comprise the amino acid sequences GGGGS (SEQ ID NO: 113) or SGGGGS (SEQ ID NO: 114). In one aspect, the peptide linker comprises or consists of (GGGGS)2 (SEQ ID NO: 115). In some instances, one of the target antigen binding domains is fused (e.g. via a peptide linker) to the CD3 antigen binding domain.

Additionally described herein are multispecific antibodies comprising a CD8 antigen binding region, in addition to the antigen binding arms of the ‘2+1’ bispecific antibody. Such antibodies may be a trispecific, tetravalent antibody: one CD3 antigen binding domain binds CD3 monovalently, two target antigen binding domains bind a target (e.g. a TAA) bivalently, while the CD8 antigen binding domain (e.g. VHH) binds CD8 monovalently.

In some instances, the CD8 antigen binding domain is a single domain antibody, such as a heavy chain variable (VH) domain that lacks a CH1 and a light chain. The heavy-chain variable domain derived from a heavy-chain antibody that naturally lacks a light chain is referred to as VHH herein to distinguish it from the conventional VH of a four-chain immunoglobulin. This VHH molecule can be derived from antibodies produced in camelidae species such as camels, alpacas, dromedaries, llamas, and guanaco. Species other than camelidae can also produce heavy-chain antibodies that naturally lack a light chain, and such VHHs are also encompassed.

Similar with other non-human antibody fragments, the amino acid sequence of the camelidae VHH can be altered recombinantly to obtain a sequence that more closely mimics a human sequence, i.e., “humanized”, thereby reducing the antigenicity of the camelidae VHH to humans. In addition, key elements derived from the camelidae VHH can also be transferred to the human VH domain to obtain a camelized human VH domain.

The CD8 antigen binding domain (e.g. VHH) may be fused to any of the other antigen binding domains present in the antibody, typically via a peptide linker. Suitable peptide linkers are known in the art and may consist of 5 to 100 amino acids, 5 to 50 amino acids, 5 to 25 amino acids, or 5 to 15 amino acids. Peptide linkers are formed mainly from glycine and serine amino acid residues and can comprise the amino acid sequences GGGGS (SEQ ID NO: 113) or SGGGGS (SEQ ID NO: 114). In one example, the peptide linker comprises or consists of (GGGGS)2 (SEQ ID NO: 115). In some instances, the CD8 antigen binding domain is fused to the one of the target antigen binding domains.

In some aspects, the CD8 antigen binding domain (e.g. VHH) comprises the following complementarity determining regions (CDRs):
i. HCDR1 having the amino acid sequence of SEQ ID NO: 109, 116 or 117;
ii. HCDR2 having the amino acid sequence of SEQ ID NO: 110;
iii. HCDR3 having the amino acid sequence of SEQ ID NO: 111;

In some aspects, the first antigen binding arm comprises a VH region comprising an amino acid sequence having at least 70% sequence identity, or at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 112, 118, 119 or 120.

The VHH proteins of SEQ ID NOs: 118-120 are derivatives of SEQ ID NO: 112, which have been modified to remove deamidation sites, including in the CDR1 sequence (see modified CDR1 sequences of SEQ ID NOs: 116 and 117, which are derivatives of SEQ ID NO: 109). Thus these modified VHHs may display improved stability compared to the original VHH of SEQ ID NO: 112.

Additionally described herein are multispecific antibodies that are trispecific, trivalent antibodies. In this format, the antibody contains three antigen binding domains that each bind a different epitope monovalently. Accordingly, in some instances, the antibody comprises the CD3 antigen binding domain described herein, the target (e.g. TAA) antigen binding domain and a third antigen binding domain. The third antigen binding domain may be a CD8 antigen binding domain, or may be a target (e.g. TAA) antigen binding domain that binds a different target (e.g. TAA) to the target antigen binding domain. In this trispecific format, each antigen binding domain comprises a light chain, a VH and CH1 (i.e. at least one constant and one variable domain of each of the and light chain), where the light chain is disulfide linked to the CH1 (i.e. each antigen binding domain is an antigen binding arm).

In some aspects, one of the antigen binding arms of an Antigen 1/Antigen 2/CD3 TriMab is capable of binding to an epitope on Antigen 1 or Antigen 2 that does not induce cell cytotoxicity. In some aspects, Antigen 1 binding arm of an Antigen 1/Antigen 2/CD3 TriMab will induce cell cytotoxicity and Antigen 2 binding arm will act as an anchoring arm with no ability to induce cell cytotoxicity in cells expressing only Antigen 2. In some aspects, Antigen 2 binding arm of an Antigen 1/Antigen 2/CD3 TriMab will induce cell cytotoxicity and Antigen 1 binding arm will act as an anchoring arm with no ability to induce cell cytotoxicity in cells expressing only Antigen 1. The inability of the anchoring arm to induce cell cytotoxicity may be, for example, due to binding to a membrane distal epitope that prevents the ability to form an active immunological synapse.

The newly identified lambda charge pairs could be used in conjunction with kappa charge pairs to produce trispecific antibodies containing three different antigen binding arms. In particular, it was recognized that:
efficient pairing of a first antigen binding arm could be achieved by using a lambda charge pair between the CH1 and the CLλ of the first antigen binding arm (e.g. the CD3 antigen binding arm described herein);
efficient pairing of a second antigen binding arm could be achieved by using a kappa charge pair between the CH1 and the CLκ of the second antigen binding arm (e.g. the target antigen binding arm described herein); and
efficient pairing of a third antigen binding arm could be achieved by using a kappa charge pair between the CH1 and the CLκ of the third antigen binding arm (e.g. a CD8 target antigen binding arm, or a target (e.g. TAA) antigen binding domain that binds a different target than the second antigen binding arm), where the charged amino acid residues are in the opposite arrangement to that of the second antigen binding arm.

Having the kappa charge pairs in the opposite arrangement to that of the second antigen binding arm means that if the kappa charge pair of second antigen binding arm contains a positively charged amino acid residue on the CH1 and a negatively charged amino acid residue on the CLκ, then the kappa charge pair of the third antigen binding arm contains a negatively charged residue on the CH1 and a positively charged amino acid residue on the CLκ.

### Constant Regions

As described herein, the antigen binding domains (e.g. the VH and VL regions defined above) may additionally comprise constant domains from a heavy and light chain. An antigen binding domain that further comprises a CH1 and a constant light chain, where the constant light chain is disulfide linked to the CH1 is also referred to herein as a “antigen binding arm” or a “Fab region”.

In some instances, the antibody described herein comprises one or more immunoglobulin heavy chain constant (CH) region. In some instances the CH is, or is derived from, the heavy chain constant sequence of an IgG (e.g. IgG1, IgG2, IgG3, IgG4), IgA (e.g. IgA1, IgA2), IgD, IgE or IgM.

IgG heavy chains are composed of a heavy variable (VH) region and three heavy constant regions (CH1, CH2 and CH3), with an additional “hinge region” between CH1 and CH2. An example of an IgG1 CH1 region amino acid sequence is provided as SEQ ID NO: 101. An example of an IgG1 CH2 amino acid sequence is provided as SEQ ID NO: 102. An example of an IgG1 CH3 amino acid sequence is provided as SEQ ID NO: 103. An example of a heavy chain amino acid sequence comprising a CH1, hinge, CH2 and CH3 is provided as SEQ ID NO: 104.

Each antigen binding domain may further comprise additional heavy chain regions, i.e. one or more of CH1, hinge, CH2 and CH3. In some cases, the antigen binding domain comprises a complete heavy chain (i.e. a VH, CH1, hinge, CH2 and CH3).

In some cases, the antigen binding domain comprises a CH1 region having an amino acid sequence having at least 70% sequence identity, or at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 101.

In some cases, the antigen binding domain comprises a CH2 region having an amino acid sequence having at least 70% sequence identity, or at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 102.

In some cases, the antigen binding domain comprises a CH3 region having an amino acid sequence having at least 70% sequence identity, or at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 103.

In some cases, the antigen binding domain comprises a heavy chain constant region having an amino acid sequence having at least 70% sequence identity, or at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 104.

In some cases, the antibody described herein comprises an immunoglobulin light chain constant (CL) region or a fragment thereof. In some cases, the CL region comprises a lambda constant (CLλ) region, e.g. having at least 70% sequence identity, or at least 75%, 80%, 85%, 86%, 87%, 88%, 89%, 90%, 91%, 92%, 93%, 94%, 95%, 96%, 97%, 98%, 99%, or 100%, sequence identity to SEQ ID NO: 105. In some cases, the CL region comprises a kappa constant (CLκ) region, e.g. having at least 70% sequence identity, or at least 75%, 80%, 85%, 86%, 87%, 88%, 89%, 90%, 91%, 92%, 93%, 94%, 95%, 96%, 97%, 98%, 99%, or 100%, sequence identity to SEQ ID NO: 106.

In some cases, the antibody comprises a complete light chain that comprises or consists of a VL region as described herein and a CL region as described herein. For example, the antibody may comprise a heavy chain containing the VL region of a CD3 antigen binding domain described herein and a CL region as described herein (e.g. a CLλ or CLκ region).

The CH, CL, heavy chain and/or light chain of the antibodies described herein may comprise one or more modifications, for example to abrogate or reduce Fc effector functions, promote formation of a heterodimeric antibody molecule, to increase the efficacy of cognate heavy and light chain pairing, and/or to assist with conjugate formation as described in more detail below.

### Charge Pairs

In some instances, an antigen binding arm (“Fab region”) of the antibody described herein comprises a charge pair, located in a light chain region (e.g. constant light chain region) and the other in a heavy chain region (e.g. constant heavy chain region 1 (CH1)) of an antigen binding arm, located at positions intended to promote association of the light and heavy chains. By “lambda charge pair”, it is meant a charge pair where a positively or negatively charged amino acid residue is located in a lambda light chain. By “kappa charge pair”, it is meant a charge pair where a positively or negatively charged amino acid residue in the light chain is located in a kappa light chain.

Without wishing to be bound by theory, it is believed that the oppositely charged amino acid residues in the charge pair increase the attraction of the heavy chain to the light chain in an antigen binding arm, thereby promoting formation of the antigen binding arm with the correct heavy and light chain.

In some aspects, at least one of the amino acid residues of the charge pair have been engineered into the antigen binding arm (i.e. at least one amino acid residue in the pair is not a wild-type amino acid residue). In some aspects, both amino acid residues in the charge pair are engineered into the antigen binding arm (i.e. both amino acid residues in the pair are not wild-type amino acid residues).

The amino acid residues of the charge pair are typically naturally occurring. Naturally occurring positively charged amino acid residues according to the present disclosure include arginine, lysine and histidine. Naturally occurring negatively charged amino acid residues according to the present disclosure include glutamic acid, serine, threonine and aspartic acid. Although serine and threonine are often described in the art as ‘uncharged’, they have an isoelectric point below 6 and therefore are partially negatively charged at neutral pH. For the purposes of the charge pairs described herein, serine and threonine are examples of negatively charged amino acid residues (together with glutamic acid and aspartic acid). Hence, a charge pair may comprise a positively charged amino acid residue selected from arginine, lysine or histidine located at one of the positions in the charge pair and a negatively charged amino acid residue selected from aspartic acid, glutamic acid, serine or threonine located at the other position in the charge pair. For example, the charge pair may comprise any one of the following pairs of amino acid residues:
arginine and aspartic acid;
arginine and glutamic acid;
arginine and serine;
arginine and threonine;
lysine and aspartic acid;
lysine and glutamic acid;
lysine and serine;
lysine and threonine;
histidine and aspartic acid;
histidine and glutamic acid;
histidine and serine; and
histidine and threonine

In some instances, the positively charged amino acid residue in the charge pair is located on the light chain and the negatively charged amino acid residue in the charge pair is located on the heavy chain. In other instances, the negatively charged amino acid residue is located on the light chain and the positively charged amino acid residue in the charge pair is located on the heavy chain. Charge pairs can be introduced at several positions to improve pairing of the correct light and heavy chains in the antigen binding arm.

In some instances, the antigen binding arm (e.g. CD3 antigen binding arm) comprises a lambda charge pair. As demonstrated herein, amino acid residues at the interface between a lambda LC and HC where charge pairs could be introduced were identified, demonstrating that the introduction of these lambda charge pairs could advantageously improve chain pairing beyond what was achieved in a previous antibody format that lacked a lambda charge pair. In some instances, the lambda charge pair comprises a positively or negatively charged amino acid residue at position 117, 119, 134, 136 or 178 of the constant light chain lambda region (CLλ). In some aspects, the lambda charge pair comprises a positively or negatively charged amino acid residue at position 141, 185, 128, 145, 183, 185, 173, or 187 of the CH1, wherein the numbering is according to EU numbering.

In some instances, the lambda charge pair is located at one or more of the following pairs of positions:
(i) position 117 in the CLλ and position 141 in the CH1;
(ii) position 117 in the CLλ and position 185 in the CH1;
(iii) position 119 in the CLλ and position 128 in the CH1;
(iv) position 134 in the CLλ and position 128 in the CH1;
(v) position 134 in the CLλ and position 145 in the CH1;
(vi) position 134 in the CLλ and position 183 in the CH1;
(vii) position 136 in the CLλ and position 185 in the CH1;
(viii) position 178 in the CLλ and position 173 in the CH1; and
(ix) position 117 in the CLλ and position 187 in the CH1.

In some instances, the lambda charge pair is located at charge pair is located at position 117 in the CLλ and position 141 in the CH1. For example, the lambda charge pair can be selected from the following list:
a. arginine at position 117 of the CLλ and aspartic acid at position 141 of the CH1;
b. arginine at position 117 of the CLλ and glutamic acid at position 141 of the CH1;
c. arginine at position 117 of the CLλ and serine at position 141 of the CH1;
d. arginine at position 117 of the CLλ and threonine at position 141 of the CH1;
e. lysine at position 117 of the CLλ and aspartic acid at position 141 of the CH1;
f. lysine at position 117 of the CLλ and glutamic acid at position 141 of the CH1;
g. lysine at position 117 of the CLλ and serine at position 141 of the CH1; and
h. lysine at position 117 of the CLλ and threonine at position 141 of the CH1.

In some instances, the lambda charge pair is selected from any one of a. to f. of the above list. In some aspects, the lambda charge pair is selected from any one of a. to e. of the above list. In some aspects, the lambda charge pair is selected from any one of a., b., and e. of the above list. In some aspects, the lambda charge pair is a.

In some instances, the lambda charge pair is located at charge pair is located at position 117 in the CLλ and position 185 in the CH1. For example, the lambda charge pair can be selected from the following list:
a. arginine at position 117 of the CLλ and aspartic acid at position 185 of the CH1;
b. arginine at position 117 of the CLλ and glutamic acid at position 185 of the CH1;
c. arginine at position 117 of the CLλ and serine at position 185 of the CH1;
d. arginine at position 117 of the CLλ and threonine at position 185 of the CH1;
e. lysine at position 117 of the CLλ and aspartic acid at position 185 of the CH1;
f. lysine at position 117 of the CLλ and glutamic acid at position 185 of the CH1;
g. lysine at position 117 of the CLλ and serine at position 185 of the CH1; and
h. lysine at position 117 of the CLλ and threonine at position 185 of the CH1.

In some instances, the lambda charge pair is located at charge pair is located at position 119 in the CLλ and position 128 in the CH1. For example, the lambda charge pair can be selected from the following list:
a. arginine at position 119 of the CLλ and aspartic acid at position 128 of the CH1;
b. arginine at position 119 of the CLλ and glutamic acid at position 128 of the CH1;
c. arginine at position 119 of the CLλ and serine at position 128 of the CH1;
d. arginine at position 119 of the CLλ and threonine at position 128 of the CH1;
e. lysine at position 119 of the CLλ and aspartic acid at position 128 of the CH1;
f. lysine at position 119 of the CLλ and glutamic acid at position 128 of the CH1;
g. lysine at position 119 of the CLλ and serine at position 128 of the CH1; and
h. lysine at position 119 of the CLλ and threonine at position 128 of the CH1.

In some aspects, the lambda charge pair is located at charge pair is located at position 134 in the CLλ and position 128 in the CH1. For example, the lambda charge pair can be selected from the following list:
a. arginine at position 134 of the CLλ and aspartic acid at position 128 of the CH1;
b. arginine at position 134 of the CLλ and glutamic acid at position 128 of the CH1;
c. arginine at position 134 of the CLλ and serine at position 128 of the CH1;
d. arginine at position 134 of the CLλ and threonine at position 128 of the CH1;
e. lysine at position 134 of the CLλ and aspartic acid at position 128 of the CH1;
f. lysine at position 134 of the CLλ and glutamic acid at position 128 of the CH1;
g. lysine at position 134 of the CLλ and serine at position 128 of the CH1; and
h. lysine at position 134 of the CLλ and threonine at position 128 of the CH1.

In some instances, the lambda charge pair is located at charge pair is located at position 134 in the CLλ and position 145 in the CH1. For example, the lambda charge pair can be selected from the following list:
a. arginine at position 134 of the CLλ and aspartic acid at position 145 of the CH1;
b. arginine at position 134 of the CLλ and glutamic acid at position 145 of the CH1;
c. arginine at position 134 of the CLλ and serine at position 145 of the CH1;
d. arginine at position 134 of the CLλ and threonine at position 145 of the CH1;
e. lysine at position 134 of the CLλ and aspartic acid at position 145 of the CH1;
f. lysine at position 134 of the CLλ and glutamic acid at position 145 of the CH1;
g. lysine at position 134 of the CLλ and serine at position 145 of the CH1; and
h. lysine at position 134 of the CLλ and threonine at position 145 of the CH1.

In some instances, the lambda charge pair is located at charge pair is located at position 134 in the CLλ and position 183 in the CH1. For example, the lambda charge pair can be selected from the following list:
a. arginine at position 134 of the CLλ and aspartic acid at position 183 of the CH1;
b. arginine at position 134 of the CLλ and glutamic acid at position 183 of the CH1;
c. arginine at position 134 of the CLλ and serine at position 183 of the CH1;
d. arginine at position 134 of the CLλ and threonine at position 183 of the CH1;
e. lysine at position 134 of the CLλ and aspartic acid at position 183 of the CH1;
f. lysine at position 134 of the CLλ and glutamic acid at position 183 of the CH1;
g. lysine at position 134 of the CLλ and serine at position 183 of the CH1; and
h. lysine at position 134 of the CLλ and threonine at position 183 of the CH1.

In some instances, the lambda charge pair is a lysine at position 134 of the CLλ, and an aspartic acid or a serine at position 183 of the CH1.

In some instances, the lambda charge pair is located at charge pair is located at position 136 in the CLλ and position 185 in the CH1. For example, the lambda charge pair can be selected from the following list:
a. arginine at position 136 of the CLλ and aspartic acid at position 185 of the CH1;
b. arginine at position 136 of the CLλ and glutamic acid at position 185 of the CH1;
c. arginine at position 136 of the CLλ and serine at position 185 of the CH1;
d. arginine at position 136 of the CLλ and threonine at position 185 of the CH1;
e. lysine at position 136 of the CLλ and aspartic acid at position 185 of the CH1;
f. lysine at position 136 of the CLλ and glutamic acid at position 185 of the CH1;
g. lysine at position 136 of the CLλ and serine at position 185 of the CH1; and
h. lysine at position 136 of the CLλ and threonine at position 185 of the CH1.

In some instances, the lambda charge pair is located at charge pair is located at position 178 in the CLλ and position 173 in the CH1. For example, the lambda charge pair can be selected from the following list:
a. arginine at position 178 of the CLλ and aspartic acid at position 173 of the CH1;
b. arginine at position 178 of the CLλ and glutamic acid at position 173 of the CH1;
c. arginine at position 178 of the CLλ and serine at position 173 of the CH1;
d. arginine at position 178 of the CLλ and threonine at position 173 of the CH1;
e. lysine at position 178 of the CLλ and aspartic acid at position 173 of the CH1;
f. lysine at position 178 of the CLλ and glutamic acid at position 173 of the CH1;
g. lysine at position 178 of the CLλ and serine at position 173 of the CH1; and
h. lysine at position 178 of the CLλ and threonine at position 173 of the CH1.

In some instances, the antigen binding arm comprising a lambda charge pair comprises more than one lambda charge pair. For example, the antigen binding arm may comprise two, three, four, five, six, seven, eight or nine lambda charge pairs at positions (i) to (ix) described above.

In some instances, an antigen binding arm in the antibody comprises a kappa charge pair. Kappa charge pairs can be introduced at several positions to improve pairing of the correct light and heavy chains in the antigen binding arm. As described above, kappa charge pairs refer to a positively charged amino acid residue and a negatively charged amino acid residue, one of which is located in the kappa light chain (e.g. CLκ) and the other in the heavy chain (e.g. CH1) of an antigen binding arm, located at positions intended to promote association of the light chain and CH1 of the antigen binding arm.

In some instances, the antigen binding arm comprises a kappa charge pair located at position 133 in the CLκ and position 183 in the CH1. In some instances, the negatively charged amino acid residue in the kappa charge pair is at position 133 of the CLκ and the positively charged amino acid residue in the kappa charge pair 183 of the CH1. In other instances, the positively charged amino acid residue in the kappa charge pair is at position 133 of the CLκ and the negatively charged amino acid residue in the kappa charge pair 183 of the CH1. In some instances, the negatively charged amino acid residue (e.g. at position 133 of the CLκ) is a glutamic acid, and wherein the positively charged amino acid residue (e.g. at position 183 of the CH1) is a lysine. As noted elsewhere, this numbering is according to EU numbering.

In some instances, one of the antigen binding arms in the antibody comprises a lambda charge pair and another (different) antigen binding arm in the antibody comprises a kappa charge pair. As exemplified herein, the CD3 antigen binding arm may comprise a lambda charge pair and the target antigen binding arm (e.g. the TAA antigen binding arm) comprises a kappa charge pair. Alternatively, the target antigen binding arm (e.g. the TAA antigen binding arm) may comprise a lambda charge pair and the CD3 antigen binding arm may comprise a kappa charge pair.

Reference herein to the singular (e.g. “a” or “the”) charge pair or domain also encompasses multiple charge pairs or multiple domains, unless context clearly dictates otherwise.

The charge pairs described herein may be combined with other strategies for promoting heterodimerization in order to further increase the correct pairing of heavy and light chain polypeptides.

Non-limiting examples of strategies for promoting heterodimerization are described in more detail below and include using disulfide engineering at the CH1/CL interface, introducing additional charge pairs (e.g. kappa charge pairs) and Fc region modifications such as knobs-into-holes and allow fractionated purification strategies.

### Fc Region Modifications

In some instances, the antibodies described herein comprise one or more modifications in one or more of the CH1, CH2 and CH3 domains that promotes formation of a heterodimeric antibody molecule by facilitating formation of the Fc regions present in the antibody. This may involve a Knobs into Holes (KiH) strategy based on single amino acid substitutions in the CH3 domains that promote heavy chain heterodimerization as described in Ridgway, 1996. The knob variant heavy chain CH3 has a small amino acid that has been replaced with a larger one, thereby generating a protuberance (knob) on the surface of said CH3 domain, and the hole variant has a large amino acid has replaced with a smaller one thereby generating a cavity (hole) on the surface of said CH3 domain. Additional modifications may also be introduced to stabilize the association between the heavy chains.

CH3 modifications to enhance heterodimerization include, for example, “hole” mutations Y407V/T366S/L368A on one Fc region and “knob” mutation T366W on the other Fc region. These may further include stabilizing cystine mutations Y349C (e.g. on the Fc region with the “hole” mutation) and stabilizing S354C mutation on the other Fc region (e.g. on the Fc region with the “knob” mutation”.

Accordingly, in one instance, the substitution to generate a knob is a substitution to tryptophan at position 366 and the substitution to generate a hole is one or more of the following:
i) a substitution to valine at position 407;
ii) a substitution to serine at position 366; and
iii) a substitution to alanine at position 368.

For example, the one Fc region may include a modification to allow fractionated elution by protein A chromatography as described in Tustian, 2016. Briefly, one of the Fc regions may comprise a modification that ablates binding to protein A (termed Fc*), allowing for selective purification of the heterodimeric FcFc* bispecific product. Examples of suitable modifications for generating an Fc* region include substitution of H435 with arginine and Y436 with phenylalanine.

Other Fc modifications that can be used in addition to those used for enhancing heterodimerization are those that reduce or abrogate binding of the antibody molecule to one or more Fcγ receptors, such as FcγRI, FcγRIIa, FcγRIIb, FcγRIII and/or to complement. Such mutations reduce or abrogate Fc effector functions. Mutations that reduce or abrogate binding of an antibody molecule to one or more Fcγ receptors and/or complement are known and include the “triple mutation” or “TM” of L234F/L235E/P331S (according to European Union numbering convention) described for example in Organesyan et al., Acta Crystallogr D Biol Crystallogr 64(6): 700-704, 2008.

In some aspects, the CH2 domain of either or both immunoglobulin heavy chain constant domains comprises the following substitutions: E233P/L234V/L235A/G236del/S267K. This combination of mutations may be referred to herein as the “Fc effector null mutation”.

Other suitable Fc region amino acid substitutions or modifications are known in the art and include, for example, the triple substitution methionine (M) to tyrosine (Y) substitution in position 252, a serine (S) to threonine (T) substitution in position 254, and a threonine (T) to glutamic acid (E) substitution in position 256, numbered according to the EU index as in Rabat (M252Y/S254T/T256E; referred to as “YTE” or “YTE mutation”) (see, e.g., U.S. Pat. No. 7,658,921; U.S. Patent Application Publication 2014/0302058; and Yu et al., Antimicrob. Agents Chemother., 61(1): e01020-16 (2017), each of which is herein incorporated by reference in its entirety). This combination of mutations may extend the half-life of the antibody.

The triple mutation, Fc effector null mutation and YTE mutation, when present, may be present in one or both heavy chain constant domains. Typically, if included, they are included in both heavy chain constant domains.

In some aspects the Fc region comprises the YTE mutation and the triple mutation. In other aspects the Fc region comprises the YTE mutation and the Fc effector null mutation.

### Engineered Disulfides

In some instances, the antibodies contain engineered disulfides. By “engineered disulfides” it is meant that a native inter-chain disulfide bond at the CH1-CL interface (e.g. at 220 of the CH1 and 212 of the LC) of the antigen binding arm, and optionally one or more additional antigen binding arms, has been replaced by an engineered (non-native) interchain disulfide. An engineered disulfide is typically formed by engineering cysteines into the CL of a light chain and the CH1 of the corresponding heavy chain and replacing the cysteines that normally form the interchain disulfide. Disclosure related to the introduction of engineered disulfide into antibodies for the purpose of promoting heterodimerization can be found e.g., in U.S. Pat. No. 9,527,927, which is herein incorporated by reference in its entirety.

In some instances, the disulfide link between the light chain and CH1 in at least one of the antigen binding arms is formed between a pair of cysteines engineered into the light chain and CH1 of that antigen binding arm.

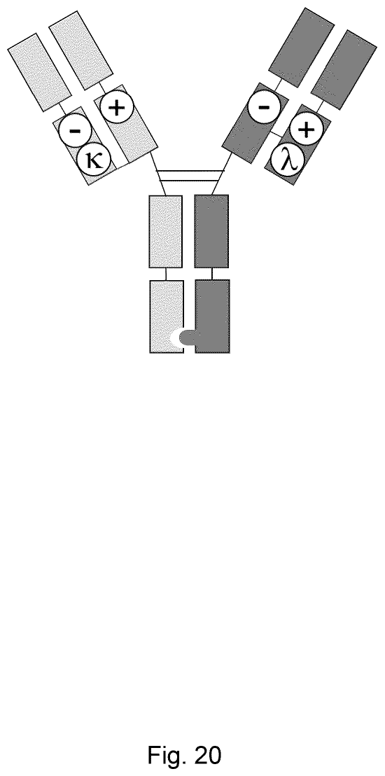

In some instances:
(i) the disulfide link between the light chain and CH1 in the CD3 antigen binding arm is formed between a pair of cysteines engineered into the light chain and the CH1 of the CD3 antigen binding arm, and the disulfide link between the light chain and CH1 in the target antigen binding arm is formed between a pair of native cysteines. A schematic of this example is provided in FIG. 20; or
(ii) the disulfide link between the light chain and CH1 in the target antigen binding arm is formed between a pair of cysteines engineered into the light chain and the CH1 of the target antigen binding arm, and the disulfide link between light chain and CH1 in the CD3 antigen binding arm is formed between a pair of native cysteines.

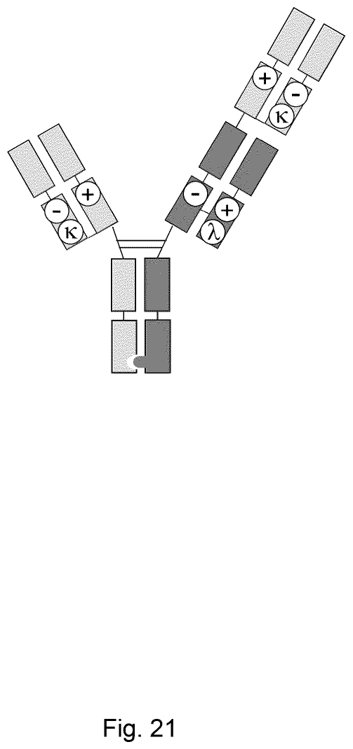

In some instances, where the antibody is in a ‘2+1’ format described herein:
(i) the disulfide link between the light chain and CH1 in the CD3 antigen binding arm is formed between a pair of cysteines engineered into the light chain and the CH1 in the CD3 antigen binding arm, and the disulfide links between the light chains and CH1 in the two target antigen binding arms are formed between pairs of native cysteines. A schematic of this example is provided in FIG. 21;
(ii) the disulfide links between the light chains and CH1 in the two target antigen binding arms are formed between pairs of cysteines engineered into the light chains and CH1 in the two target antigen binding arms, and the disulfide link between the light chain and CH1 in the CD3 antigen binding arm is formed between a pair of native cysteines; or
(iii) all three antigen binding arms contain engineered disulfides, but the cysteines engineered into the CD3 antigen binding arm are at different positions (e.g. in the light chain) to the cysteines engineered into the target antigen binding arms.

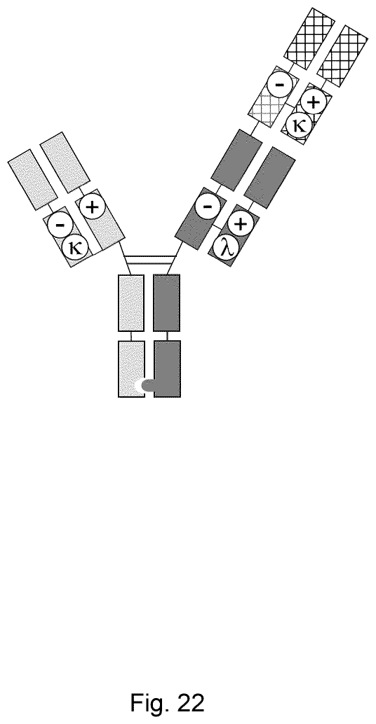

In some instances, where the antibody is in a trispecific format described herein the disulfide link between the light chain and CH1 of the first antigen binding arm (e.g. the CD3 antigen binding arm) is formed between a pair of cysteines engineered into the light chain and CH1 of the first antigen binding arm, the disulfide link formed between the light chain and CH1 of the second antigen binding arm (e.g. the target antigen binding arm) is formed between a pair of native cysteines of the second antigen binding arm, and the disulfide link formed between the light chain and CH1 of the third antigen binding arm (e.g. a CD8 target antigen binding arm, or a target antigen binding domain that binds a different target than the second antigen binding arm) is formed between a pair of cysteines engineered into the light chain and CH1 of the first antigen binding arm. A schematic of this example is provided in FIG. 22.

In cases where both the first and third antigen binding arms contain engineered disulfides, it is desirable that the engineered disulfide introduced into the third antigen binding arm is different to the engineered disulfide introduced in the first antigen binding arm as this is believed to further improve correct pairing. Hence, optionally the pair of cysteines inserted into the third antigen binding arm are at different amino acid residue positions to the pair of cysteines inserted into the first antigen binding arm

As described above, the light chains may comprise a CLλ or a CLκ. In some instances, the pair of cysteines engineered into the CLλ and CH1 are located at position 122 of the CLλ and position 126 of the CH1, and wherein the same CLλ comprises a non-cysteine residue at position 212 and the same CH1 comprises a non-cysteine residue at position 220. In some instances, the non-cysteine residues are valines.

An exemplary amino acid sequence of a CLλ comprising an engineered cysteine is provided as SEQ ID NO: 107 and an exemplary amino acid sequence of the CH1 comprising the corresponding engineered cysteine to form the engineered disulfide is provided as SEQ ID NO: 108.

In some instances, the pair of cysteines engineered into a constant light chain kappa region (CLκ) and CH1 are located at position 121 of the CLκ and position 126 of the CH1, and wherein the same CLκ comprises a non-cysteine residue at position 214 and the same CH1 comprises a non-cysteine residue at position 220. In some instances, the non-cysteine residues are valines.

In some instances, the antibody is in a ‘DuetMab’ format, e.g. as described in U.S. Pat. No. 9,527,927. A DuetMab includes engineered disulfides to enhance correct heavy/light chain pairing and Fc region modifications to enhance correct heterodimerization of heavy chains.

In some aspects, the antibody comprises:
a CD3 antigen binding arm as described above comprising a first Fc region; and
a target antigen binding arm as described above comprising a second Fc region,
wherein the first and second Fc regions comprise modifications to facilitate heterodimerization of the first and second Fc regions.

In some aspects, the antibody comprises:
a CD3 antigen binding arm as described above comprising a first Fc region; and
a target antigen binding arm as described above comprising a second Fc region,
wherein the first and second Fc regions comprise modifications to facilitate heterodimerization of the first and second Fc regions, and wherein the antibody comprises engineered disulfides.

In some aspects, the antibody comprises:
a CD3 antigen binding arm comprising a lambda charge pair as described above and a first Fc region; and
a target antigen binding arm as described above comprising a second Fc region,
wherein the first and second Fc regions comprise modifications to facilitate heterodimerization of the first and second Fc regions.

In some aspects, the antibody comprises:
a CD3 antigen binding arm comprising a lambda charge pair as described above and a first Fc region; and
a target antigen binding arm as described above comprising a second Fc region,
wherein the first and second Fc regions comprise modifications to facilitate heterodimerization of the first and second Fc regions, and wherein the antibody comprises engineered disulfides.

In some aspects, the antibody comprises:
a CD3 antigen binding arm comprising a lambda charge pair as described above and a first Fc region; and
a target antigen binding arm comprising a kappa charge pair as described above and a second Fc region,
wherein the first and second Fc regions comprise modifications to facilitate heterodimerization of the first and second Fc regions.

In some aspects, the multispecific antibody comprises:
a CD3 antigen binding arm comprising a lambda charge pair as described above and a first Fc region; and
a target antigen binding arm comprising a kappa charge pair as described above and a second Fc region,
wherein the first and second Fc regions comprise modifications to facilitate heterodimerization of the first and second Fc regions, and wherein the multispecific antibody comprises engineered disulfides.

Non-limiting examples of antibodies comprising a lambda charge pair, a kappa charge pair, engineered disulfides and modifications to facilitate heterodimerization of the first and second Fc regions are provided in the examples.

### Sequence Identity and Mutations

In some instances, the CLλ described herein comprises an amino acid sequence having at least 90%, 91%, 92%, 93%, 94%, 95%, 96%, 97%, 98%, or 99% identity to SEQ ID NO: 105 or SEQ ID NO: 107. In some instances, the CLλ comprises an amino acid sequence of SEQ ID NO: 105 or SEQ ID NO: 107 with 1, 2, 3, 4, 5, 6, 7, 8, 9 or 10 amino acid modifications.

In some instances, the CLκ described herein comprises an amino acid sequence having at least 90%, 91%, 92%, 93%, 94%, 95%, 96%, 97%, 98%, or 99% identity to SEQ ID NO: 106. In some instances, the CLκ comprises an amino acid sequence of SEQ ID NO: 106 with 1, 2, 3, 4, 5, 6, 7, 8, 9 or 10 amino acid modifications.

In some instances, the CH1 comprises an amino acid sequence having at least 90%, 91%, 92%, 93%, 94%, 95%, 96%, 97%, 98%, 99%, or 100% identity to SEQ ID NO: 101 or SEQ ID NO: 108. In some instances, the CH1 comprises an amino acid sequence of SEQ ID NO: 101 or SEQ ID NO: 108 with 1, 2, 3, 4, 5, 6, 7, 8, 9 or 10 amino acid modifications.

In some instances, the CH2 comprises an amino acid sequence having at least 90%, 91%, 92%, 93%, 94%, 95%, 96%, 97%, 98%, 99%, or 100% identity to SEQ ID NO: 102. In some instances, the CH2 comprises an amino acid sequence of SEQ ID NO: 102 with 1, 2, 3, 4, 5, 6, 7, 8, 9 or 10 amino acid modifications.

In some instances, the CH3 comprises an amino acid sequence having at least 90%, 91%, 92%, 93%, 94%, 95%, 96%, 97%, 98%, 99%, or 100% identity to SEQ ID NO: 103. In some instances, the CH3 comprises an amino acid sequence of SEQ ID NO: 103 with 1, 2, 3, 4, 5, 6, 7, 8, 9 or 10 amino acid modifications.

The 1, 2, 3, 4, 5, 6, 7, 8, 9 or 10 amino acid modifications in the constant regions (CLλ, CLκ, CH1, CH2 and CH3 may be in addition to the modifications described above to introduce the charge pairs, engineered disulfides and/or Fc region modifications described above. For example, compared to the wild type CLλ set forth in SEQ ID NO: 105, the CLλ used in the antibody may contain a lambda charge pair mutation, an engineered disulfide (e.g. S122C and C212V) and 1, 2, 3, 4, 5, 6, 7, 8, 9 or 10 further amino acid modifications. As another example, compared to the wild type CH1 provided in SEQ ID NO: 101, a CH1 used in the antibody may contain a lambda charge mutation, an engineered disulfide (e.g. F126C, C220V) and 1, 2, 3, 4, 5, 6, 7, 8, 9 or 10 further amino acid modifications.

In some instances, the CD3 antigen binding domain comprises a VH region having at least 90%, 91%, 92%, 93%, 94%, 95%, 96%, 97%, 98%, or 99% to the amino acid sequences defined in (16)-(24) above. In some instances, the CD3 antigen binding domain comprises a VH region having the with amino acid sequences defined in (16)-(24) above with 1, 2, 3, 4, 5, 6, 7, 8, 9 or 10 amino acid modifications.

In some instances, the CD3 antigen binding domain comprises a VL region having at least 90%, 91%, 92%, 93%, 94%, 95%, 96%, 97%, 98%, or 99% to the amino acid sequences defined in (25)-(27) above. In some instances, the CD3 antigen binding domain comprises a VL region having the with amino acid sequences defined in (25)-(27) above with 1, 2, 3, 4, 5, 6, 7, 8, 9 or 10 amino acid modifications.

The CLλ regions described herein comprises an amino acid sequence having at least 90%, 91%, 92%, 93%, 94%, 95%, 96%, 97%, 98%, or 99% identity to SEQ ID NO: 105 or SEQ ID NO: 107. In some instances, the CLλ comprises an amino acid sequence of SEQ ID NO: 105 or SEQ ID NO: 107 with 1, 2, 3, 4, 5, 6, 7, 8, 9 or 10 amino acid modifications.

In some cases, the CD3 antigen binding domain comprises a VH region according to any one of (16) to (24) below:

An amino acid modification may be an insertion, a substitution, or a deletion. In some aspects, the amino acid modification is a substitution of an amino acid residue to any other naturally occurring or non-naturally occurring amino acid residue.

Naturally occurring residues may be divided into classes based on common side chain properties:
1) nonpolar, aliphatic: glycine (G), methionine (M), alanine (A), valine (V), leucine (L), isoleucine (I);
2) polar: cysteine (C), asparagine (N), glutamine (Q), proline (P);
3) polar, partially negatively charged: serine (S), threonine (T);
4) acidic (negatively charged): aspartic acid (D), glutamic acid (E);
5) basic (positively charged): histidine (H), lysine (K), arginine I;
6) aromatic: tryptophan (W), tyrosine (Y), phenylalanine (F).

As described above, serine (S) and threonine (T) have an isoelectric point below 6 and are partially negatively charged at neutral pH, hence they are classed here as ‘polar, partially negatively charged’.

The amino acid substitution may be a conservative amino acid substitution. Conservative amino acid substitutions may involve exchange of a member of one of these classes with another member of the same class. For example, a conservative amino acid substitution may be a substitution of the acidic amino acid glutamic acid (E) for the acidic amino acid aspartic acid (D).

### Nucleic Acids, Vectors and Host Cells

Also provided herein is one or more nucleic acid(s) encoding the antibody described herein. In some instances, the nucleic acid(s) is/are purified or isolated, e.g. from other nucleic acid, or naturally-occurring biological material. The skilled person would have no difficulty in preparing such nucleic acid molecules using methods well-known in the art.

In some instances, the one or more nucleic acids encode a light chain as described herein and/or a CH1 as described herein. The one or more nucleic acid(s) encoding the CH1 may further encode other heavy chain domains, e.g. the hinge, CH2 and CH3, and may encode a complete heavy chain.

The present disclosure also provides one or more vector(s) comprising nucleic acid(s) encoding an antibody or fragment thereof described herein. Suitable vectors can be chosen or constructed, containing appropriate regulatory sequences, including promoter sequences, terminator fragments, polyadenylation sequences, enhancer sequences, marker genes and other sequences as appropriate. Preferably, the vector contains appropriate regulatory sequences to drive the expression of the nucleic acid in a host cell. Vectors may be plasmids, viral e.g. phage, or phagemid, as appropriate.

The antibody may be produced from a light chain vector and a heavy chain vector. A light chain vector may contain the nucleic acid encoding the one of the light chains in the antibody and the nucleic acid encoding the other light chain acid, which may be present on the vector as separate cassettes (e.g. each operably connected to a different promoter). Similarly, a heavy chain vector may contain may be used to encode both the CH1 (and Fc region, if present) of one antigen binding arm and the CH1 (and Fc region, if present) of the other antigen binding arm, which may be present on the vector as separate cassettes. Alternatively, separate vectors may be used to encode each of the light chains, CH1s and Fc regions (if present).

A nucleic acid molecule or vector as described herein may be introduced into a host cell. Techniques for the introduction of nucleic acid or vectors into host cells are well established in the art and any suitable technique may be employed. A range of host cells suitable for the production of recombinant antibody molecules are known in the art, and include bacterial, yeast, insect or mammalian host cells. In some aspects, the host cell is a mammalian cell, such as a CHO, NS0, or HEK cell, for example a HEK293 cell. In some aspects, the host cell is a CHO cell.

### Methods of Producing the Antibodies

Also provided herein is a method of producing the antibody described herein. Techniques for the purification of recombinant antibody molecules are well-known in the art and include, for example high performance liquid chromatography, fast protein liquid chromatography, ion exchange chromatography, and affinity chromatography, e.g. using Protein A or Protein L or by binding to an affinity tag. In some instances, purification is carried out using affinity chromatography (e.g. Protein A affinity chromatography). In some instances, purification further comprises (e.g. in addition to Protein A chromatography) light chain affinity chromatography.

The method may also comprise formulating the antibody molecule into a pharmaceutical composition, optionally with a pharmaceutically acceptable excipient or other substance as described below.

### Treatment

The antibodies described herein may be useful for therapeutic applications, such as in treatment of cancer.

An antibody as described herein may be used in a method of treatment of the human or animal body. Related aspects of the of the disclosure provide;
(i) an antibody described herein for use as a medicament,
(ii) an antibody described herein for use in a method of treatment of a disease or disorder,
(iii) an antibody described herein in the manufacture of a medicament for use in the treatment of a disease or disorder; and,
(iv) a method of treating a disease or disorder in an individual, wherein the method comprises administering to the individual a therapeutically effective amount of an antibody described herein.

The individual may be a patient, preferably a human patient.

Treatment may be any treatment or therapy in which some desired therapeutic effect is achieved, for example, the inhibition or delay of the process of the condition and includes a reduction in the rate of progress, a halt in the rate of progress, amelioration of the condition, cure or remission (whether partial or total) of the condition, preventing, ameliorating, delaying, abating or arresting one or more symptoms and/or signs of the condition or prolonging survival of an individual or patient beyond that expected in the absence of treatment.

Treatment as a prophylactic measure (i.e. prophylaxis) is also included. For example, an individual susceptible to or at risk of the occurrence or re-occurrence of a disease such as cancer may be treated as described herein. Such treatment may prevent or delay the occurrence or re-occurrence of the disease in the individual.

Whilst an antibody may be administered alone, antibodies or fragments thereof will usually be administered in the form of a pharmaceutical composition, which may comprise at least one component in addition to the antibody. Another aspect of the disclosure therefore provides a pharmaceutical composition comprising an antibody as described herein. A method comprising formulating an antibody into a pharmaceutical composition is also provided.

Pharmaceutical compositions may comprise, in addition to the antibody, a pharmaceutically acceptable excipient, carrier, buffer, stabilizer or other materials well known to those skilled in the art. The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio.

Administration may be in a “therapeutically effective amount”, this being sufficient to show benefit to an individual. The actual amount administered, and rate and time-course of administration, will depend on the nature and severity of what is being treated, the particular individual being treated, the clinical condition of the individual, the cause of the disorder, the site of delivery of the composition, the type of antibody molecule, the method of administration, and the scheduling of administration.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilized for realizing the disclosure in diverse forms thereof.

While the disclosure has been described in conjunction with the exemplary instances described above, many equivalent modifications and variations will be apparent to those skilled in the art when given this disclosure. Accordingly, the exemplary instances of the disclosure set forth above are considered to be illustrative and not limiting. Various changes to the described instances may be made without departing from the spirit and scope of the disclosure.

For the avoidance of any doubt, any theoretical explanations provided herein are provided for the purposes of improving the understanding of a reader. Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another aspect includes from the one particular value and/or to the other particular value.

Similarly, when values are expressed as approximations, by the use of the antecedent “about,” it will be understood that the particular value forms another aspect. The term “about” in relation to a numerical value is optional and means for example +/−10%.

### Sequences

| SEQ ID | | |
|---|---|---|
| NO: | Description | Sequence |
| 1 | V_{K} Parent | DIVMTQTPLSLSVTPGQPASISCKSSQSLVHNNANTYLSWYLQ |
| | | KPGQSPQSLIYKVSNRFSGVPDRFSGSGSGTDFTLKISRVEAE |
| | | DVGVYYCGQGTQYPFTFGSGTKVEIK |
| 2 | V_{K} Parent LCDR1 | KSSQSLVHNNANTYLS |
| 3 | V_{K} Parent LCDR2 | KVSNRFS |
| 4 | V_{K} Parent LCDR3 | GQGTQYPFT |
| 5 | VH Parent | QVQLVESGGGVVQPGRSLRLSCAASGFTFTKAWMHWVRQA |
| | | PGKQLEWVAQIKDKSNSYATYYADSVKGRFTISRDDSKNTLYL |
| | | QMNSLRAEDTAVYYCRGVYYALSPFDYWGQGTLVTVSS |
| 6 | VH HCDR1 | KAWMH |
| 7 | VH HCDR2 | QIKDKSNSYATYYADSVKG |
| 8 | VH HCDR3 | RGVYYALSPFDY |
| 9 | V_{K} 3 | DIVMTQSPLSLPVTPGEPASISCRSSQSLVHNQANTYLSWYLQ |
| | | KPGQSPQSLIYKVSNRFSGVPDRFSGSGSGTDFTLKISRVEAE |
| | | DVGVYYCGQGTQYPFTFGSGTKVEIK |
| 10 | V_{K} 3 LCDR1 | RSSQSLVHNQANTYLS |
| 11 | V_{K} 3 LCDR2 | KVSNRFS |
| 12 | V_{K} 3 LCDR3 | GQGTQYPFT |
| 13 | V_{K} 7 | DIVMTQSPLSLPVTPGEPASISCRSSQSLVHNTGNTYLSWYLQ |
| | | KPGQSPQSLIYKVSNRASGVPDRFSGSGSGTDFTLKISRVEAE |
| | | DVGVYYCGQGTQYPFTFGSGTKVEIK |
| 14 | V_{K} 7 LCDR1 | RSSQSLVHNTGNTYLS |
| 15 | V_{K} 7 LCDR2 | KVSNRAS |
| 16 | V_{K} 7 LCDR3 | GQGTQYPFT |
| 17 | Vₖ 11 | DIVMTQSPLSLPVTPGEPASISCRSSQSLVHNQANTYLSWYLQ |
| | | KPGQSPQSLIYKVSNRYSGVPDRFSGSGSGTDFTLKISRVEAE |
| | | DVGVYYCGQGTQYPFTFGSGTKVEIK |
| 18 | V_{K} 11 LCDR1 | RSSQSLVHNQANTYLS |
| 19 | V_{K} 11 LCDR2 | KVSNRYS |
| 20 | V_{K} 11 LCDR3 | GQGTQYPFT |
| 21 | VH 1 | EVQLVESGGALVKPGGSLRLSCAASGFTFTKAWMHWVRQAP |
| | | GKQLEWVAQIKDKSNSYATYYAESVKGRFTISRDDSKNTLYLQ |
| | | MNSLKTEDTAVYYCRGVYYALSPFDYWGQGTLVTVSS |
| 22 | VH 1 HCDR1 | KAWMH |
| 23 | VH2 HCDR2 | QIKDKSNSYATYYAESVKGR |
| 24 | VH3 HCDR3 | RGVYYALSPFDY |
| 25 | VH 7 | EVQLVESGGALVKPGGSLRLSCAASGFTFTKAYMHWVRQAP |
| | | GKQLEWVAQIKDKSNSYATYYAESVKGRFTISRDDSKNTLYLQ |
| | | MNSLKTEDTAVYYCRGVYYALSPFDYWGQGTLVTVSS |
| 26 | VH 7 HCDR1 | KAYMH |
| 27 | VH7 HCDR2 | QIKDKSNSYATYYAESVKGR |
| 28 | VH7 HCDR3 | RGVYYALSPFDY |
| 29 | VH 8 | EVQLVESGGALVKPGGSLRLSCAASGFTFSNAWMHWVRQAP |
| | | GKQLEWVAQIKDKSNSYATYYAESVKGRFTISRDDSKNTLYLQ |
| | | MNSLKTEDTAVYYCRGVYYALSPFDYWGQGTLVTVSS |
| 30 | VH 8 HCDR1 | NAWMH |
| 31 | VH 8 HCDR2 | QIKDKSNSYATYYAESVKGR |
| 32 | VH 8 HCDR3 | RGVYYALSPFDY |
| 33 | VH 9 | EVQLVESGGALVKPGGSLRLSCAASGFTFTKAWMSWVRQAP |
| | | GKQLEWVAQIKDKSNSYATYYAESVKGRFTISRDDSKNTLYLQ |
| | | MNSLKTEDTAVYYCRGVYYALSPFDYWGQGTLVTVSS |
| 34 | VH 9 HCDR1 | KAWMS |
| 35 | VH9 HCDR2 | QIKDKSNSYATYYAESVKGR |
| 36 | VH9 HCDR3 | RGVYYALSPFDY |
| 37 | VH 13 | EVQLVESGGALVKPGGSLRLSCAASGFTFTKAYMHWVRQAP |
| | | GKQLEWVAQIKDRSQSYATYYAESVKGRFTISRDDSKNTLYLQ |
| | | MNSLKTEDTAVYYCRGVYYALSPFDYWGQGTLVTVSS |
| 38 | VH 13 HCDR1 | KAYMH |
| 39 | VH 13 HCDR2 | QIKDRSQSYATYYAESVKGR |
| 40 | VH 13 HCDR3 | RGVYYALSPFDY |
| 41 | VH 21 | EVQLVESGGALVKPGGSLRLSCAASGFTFSNAYMHWVRQAP |
| | | GKQLEWVAQIKDRSQSYATYYAESVKGRFTISRDDSKNTLYLQ |
| | | MNSLKTEDTAVYYCRGVYYALSPFDYWGQGTLVTVSS |
| 42 | VH 21 HCDR1 | NAYMH |
| 43 | VH 21 HCDR2 | QIKDRSQSYATYYAESVKGR |
| 44 | VH 21 HCDR3 | RGVYYALSPFDY |
| 45 | Vₖ 17 | DIVMTQSPLSLPVTPGEPASISCRSSQSLVHNTGNTYLSWYLQ |
| | | KPGQSPQSLIYKVSNRASGVPDRFSGSGSGTDFTLKISRVEAE |
| | | DVGVYYCGQGTQAPFTFGSGTKVEIK |
| 46 | Vₖ 17 LCDR1 | RSSQSLVHNTGNTYLS |
| 47 | Vₖ 17 LCDR2 | KVSNRAS |
| 48 | Vₖ 17 LCDR3 | GQGTQAPFT |
| 49 | VH 35 | EVQLVESGGALVKPGGSLRLSCAASGFTFSNAWMHWVRQAP |
| | | GKQLEWVAQIKDRSQTYATYYAESVKGRFTISRDDSKNTLYLQ |
| | | MNSLKTEDTAVYYCRGVYYALAPFDYWGQGTLVTVSS |
| 50 | VH 35 HCDR1 | NAWMH |
| 51 | VH 35 HCDR2 | QIKDRSQTYATYYAESVKGR |
| 52 | VH 35 HCDR3 | RGVYYALAPFDY |
| 53 | VH 37 | EVQLVESGGALVKPGGSLRLSCAASGFTFTKAYMHWVRQAP |
| | | GKQLEWVAQIKDRSQSYATYYAESVKGRFTISRDDSKNTLYLQ |
| | | MNSLKTEDTAVYYCRGVYYALAPFDYWGQGTLVTVSS |
| 54 | VH 37 HCDR1 | KAYMH |
| 55 | VH 37 HCDR2 | QIKDRSQS |
| 56 | VH 37 HCDR3 | RGVYYALAPFDY |
| 57 | Vₖ 26 | DIVMTQSPLSLPVTPGEPASISCRSSQSLVHNTGNTYLSWYLQ |
| | | KPGQSPQSLIYKVSNRASGVPDRFSGSGSGTDFTLKISRVEAE |
| | | DVGVYYCGQGTQAPFSFGSGTKVEIK |
| 58 | Vₖ 26 LCDR1 | RSSQSLVHNTGNTYLS |
| 59 | Vₖ 26 LCDR2 | KVSNRAS |
| 60 | Vₖ 26 LCDR3 | GQGTQAPFS |
| 61 | Vₖ 29 | DIVMTQSPLSLPVTPGEPASISCRSSQSLVHNTGNTYLSWYLQ |
| | | KPGQSPQSLIYKVSNRASGVPDRFSGSGSGTDFTLKISRVEAE |
| | | DVGVYYCGQGTQFPFTFGSGTKVEIK |
| 62 | Vₖ 29 LCDR1 | RSSQSLVHNTGNTYLS |
| 63 | Vₖ 29 LCDR2 | KVSNRAS |
| 64 | Vₖ 29 LCDR3 | GQGTQFPFT |
| 65 | VH R75 | EVQLVESGGALVKPGGSLRLSCAASGFTFTRAWMHWVRQAP |
| | | GKQLEWVAQIKDRSQTYATYYAESVKGRFTISRDDSKNTLYLQ |
| | | MNSLKTEDTAVYYCRGVYYANAPFDYWGQGTLVTVSS |
| 66 | VH R75 HCDR1 | RAWMH |
| 67 | VH R75 HCDR2 | QIKDRSQTYATYYAESVKGR |
| 68 | VH R75 HCDR3 | RGVYYANAPFDY |
| 69 | VH R79 | EVQLVESGGALVKPGGSLRLSCAASGFTFTRAWMHWVRQAP |
| | | GKQLEWVAQIKDRSQTYATYYAESVKGRFTISRDDSKNTLYLQ |
| | | MNSLKTEDTAVYYCRGVYYAVTPFDYWGQGTLVTVSS |
| 70 | VH R79 HCDR1 | RAWMH |
| 71 | VH R79 HCDR2 | QIKDRSQTYATYYAESVKGR |
| 72 | VH R79 HCDR3 | RGVYYAVTPFDY |
| 73 | VH R82 | EVQLVESGGALVKPGGSLRLSCAASGFTFTRAWMHWVRQAP |
| | | GKQLEWVAQIKDRSQTYATYYAESVKGRFTISRDDSKNTLYLQ |
| | | MNSLKTEDTAVYYCRGVYYANTPFDYWGQGTLVTVSS |
| 74 | VH R82 HCDR1 | RAWMH |
| 75 | VH R82 HCDR2 | QIKDRSQTYATYYAESVKGR |
| 76 | VH R82 HCDR3 | RGVYYANTPFDY |
| 77 | VH SN75 | EVQLVESGGALVKPGGSLRLSCAASGFTFSNAWMHWVRQAP |
| | | GKQLEWVAQIKDRSQTYATYYAESVKGRFTISRDDSKNTLYLQ |
| | | MNSLKTEDTAVYYCRGVYYANAPFDYWGQGTLVTVSS |
| 78 | VH SN75 HCDR1 | NAWMH |
| 79 | VH SN75 HCDR2 | QIKDRSQTYATYYAESVKGR |
| 80 | VH SN75 HCDR3 | RGVYYANAPFDY |
| 81 | VH SN79 | EVQLVESGGALVKPGGSLRLSCAASGFTFSNAWMHWVRQAP |
| | | GKQLEWVAQIKDRSQTYATYYAESVKGRFTISRDDSKNTLYLQ |
| | | MNSLKTEDTAVYYCRGVYYAVTPFDYWGQGTLVTVSS |
| 82 | VH SN79 HCDR1 | NAWMH |
| 83 | VH SN79 HCDR2 | QIKDRSQTYATYYAESVKGR |
| 84 | VH SN79 HCDR3 | RGVYYAVTPFDY |
| 85 | VH SN82 | EVQLVESGGALVKPGGSLRLSCAASGFTFSNAWMHWVRQAP |
| | | GKQLEWVAQIKDRSQTYATYYAESVKGRFTISRDDSKNTLYLQ |
| | | MNSLKTEDTAVYYCRGVYYANTPFDYWGQGTLVTVSS |
| 86 | VH SN82 HCDR1 | NAWMH |
| 87 | VH SN82 HCDR2 | QIKDRSQTYATYYAESVKGR |
| 88 | VH SN82 HCDR3 | RGVYYANTPFDY |
| 89 | VH E75 | EVQLVESGGALVKPGGSLRLSCAASGFTFTEAWMHWVRQAP |
| | | GKQLEWVAQIKDRSQTYATYYAESVKGRFTISRDDSKNTLYLQ |
| | | MNSLKTEDTAVYYCRGVYYANAPFDYWGQGTLVTVSS |
| 90 | VH E75 HCDR1 | EAWMH |
| 91 | VH E75 HCDR2 | QIKDRSQTYATYYAESVKGR |
| 92 | VH E75 HCDR3 | RGVYYANAPFDY |
| 93 | VH E79 | EVQLVESGGALVKPGGSLRLSCAASGFTFTEAWMHWVRQAP |
| | | GKQLEWVAQIKDRSQTYATYYAESVKGRFTISRDDSKNTLYLQ |
| | | MNSLKTEDTAVYYCRGVYYAVTPFDYWGQGTLVTVSS |
| 94 | VH E79 HCDR1 | EAWMH |
| 95 | VH E79 HCDR2 | QIKDRSQTYATYYAESVKGR |
| 96 | VH E79 HCDR3 | RGVYYAVTPFDY |
| 97 | VH E82 | EVQLVESGGALVKPGGSLRLSCAASGFTFTEAWMHWVRQAP |
| | | GKQLEWVAQIKDRSQTYATYYAESVKGRFTISRDDSKNTLYLQ |
| | | MNSLKTEDTAVYYCRGVYYANTPFDYWGQGTLVTVSS |
| 98 | VH E82 HCDR1 | EAWMH |
| 99 | VH E82 HCDR2 | QIKDRSQTYATYYAESVKGR |
| 100 | VH E82 HCDR3 | RGVYYANTPFDY |
| 101 | IgG1 CH1 | ASTKGPSVFPLAPSSKSTSGGTAALGCLVKDYFPEPVTVSWN |
| | | SGALTSGVHTFPAVLQSSGLYSLSSVVTVPSSSLGTQTYICNV |
| | | NHKPSNTKVDKRVEPKSC |
| 102 | IgG1 CH2 | LLGGPSVFLFPPKPKDTLMISRTPEVTCVVVDVSHEDPEVKFN |
| | | WYVDGVEVHNAKTKPREEQYNSTYRVVSVLTVLHQDWLNGK |
| | | EYKCKVSNKALPAPIEKTIS |
| 103 | IgG1 CH3 | GQPREPQVYTLPPSREEMTKNQVSLTCLVKGFYPSDIAVEWE |
| | | SNGQPENNYKTTPPVLDSDGSFFLYSKLTVDKSRWQQGNVF |
| | | SCSVMHEALHNHYTQKSLSLSPGK |
| 104 | IgG1 heavy chain | ASTKGPSVFPLAPSSKSTSGGTAALGCLVKDYFPEPVTVSWN |
| | constant | SGALTSGVHTFPAVLQSSGLYSLSSVVTVPSSSLGTQTYICNV |
| | | NHKPSNTKVDKRVEPKSCDKTHTCPPCPAPELLGGPSVFLFP |
| | | PKPKDTLMISRTPEVTCVVVDVSHEDPEVKFNWYVDGVEVHN |
| | | AKTKPREEQYNSTYRVVSVLTVLHQDWLNGKEYKCKVSNKAL |
| | | PAPIEKTISKAKGQPREPQVYTLPPSREEMTKNQVSLTCLVKG |
| | | FYPSDIAVEWESNGQPENNYKTTPPVLDSDGSFFLYSKLTVDK |
| | | SRWQQGNVFSCSVMHEALHNHYTQKSLSLSPGK |
| 105 | lambda constant | GQPKAAPSVTLFPPSSEELQANKATLVCLISDFYPGAVTVA |
| | (CLλ) region | WKADSSPVKAGVETTTPSKQSNNKYAASSYLSLTPEQWKS |
| | | HRSYSCQVTHEGSTVEKTVAPTECS |
| 106 | Kappa (CLκ) | RTVAAPSVFIFPPSDEQLKSGTASVVCLLNNFYPREAKVQWKV |
| | constant region | DNALQSGNSQESVTEQDSKDSTYSLSSTLTLSKADYEKHKVY |
| | | ACEVTHQGLSSPVTKSFNRGEC |
| 107 | ‘V12’ CLλ region | GQPKAAPSVTLFPPCSEELQANKATLVCLISDFYPGAVTVA |
| | modified to form | WKADSSPVKAGVETTTPSKQSNNKYAASSYLSLTPEQWKS |
| | an engineered | HRSYSCQVTHEGSTVEKTVAPTEVS |
| | disulfide bridge | |
| 108 | ‘V12’ CH1 region | ASTKGPSVCPLAPSSKSTSGGTAALGCLVKDYFPEPVTVSW |
| | modified to form | NSGALTSGVHTFPAVLQSSGLYSLSSVVTVPSSSLGTQTYIC |
| | an engineered | NVNHKPSNTKVDKRVEPKSV |
| | disulfide bridge | |
| 109 | CD8 VHH HCDR1 | DDYAIG |
| 110 | CD8 VHH HCDR2 | CIRVSDGSTYYADSVKG |
| 111 | CD8 VHH HCDR3 | GSLYTCVQSIVWPARPYYDMDY |
| 112 | CD8 VHH | EVQLLESGGGLVQPGGSLRLSCAASGFTFDDYAIGWFRQAPG |
| | | KEREGVSCIRVSDGSTYYADSVKGRFTISRDNSKNTLYLQMNS |
| | | LRAEDTAVYYCAAGSLYTCVQSIVWPARPYYDMDYWGQGTL |
| | | VTVSS |
| 113 | Linker | GGGGS |
| 114 | Linker | SGGGGS |
| 115 | Linker | GGGGSGGGGS |
| 116 | CD8 VHH HCDR1 | TDYAIG |
| 117 | CD8 VHH HCDR1 | SDYAIG |
| 118 | CD8 VHH | EVQLLESGGGLVQPGGSLRLSCAASGFTFTDYAIGWFRQAPG |
| | | KEREGVSCIRVSDGSTYYADSVKGRFTISRDSSKNTLYLQMNS |
| | | LRAEDTAVYYCAAGSLYTCVQSIVWPARPYYDMDYWGQGTL |
| | | VTVSS |
| 119 | CD8 VHH | EVQLLESGGGLVQPGGSLRLSCAASGFTFTDYAIGWFRQAPG |
| | | KEREGVSCIRVSDGSTYYADSVKGRFTISRDNAKNTLYLQMNS |
| | | LRAEDTAVYYCAAGSLYTCVQSIVWPARPYYDMDYWGQGTL |
| | | VTVSS |
| 120 | CD8 VHH | EVQLLESGGGLVQPGGSLRLSCAASGFTFSDYAIGWFRQAPG |
| | | KEREGVSCIRVSDGSTYYADSVKGRFTISRDTSKNTLYLQMNS |
| | | LRAEDTAVYYCAAGSLYTCVQSIVWPARPYYDMDYWGQGTL |
| | | VTVSS |

### EXAMPLES

### Example 1—Material and Methods

### Cells and Media

The CD3-expressing cell lines, including HPB-ALL, HSC-F, Jurkat, Jurkat TCR KO, and EGFR-expressing cell lines, including NCI H358 and MDA-MB468, were obtained from the American Type Culture Collection. All cell lines except MDA-MB-468 cells were maintained in RPMI 1640 media supplied with 10% heat-inactivated FBS and cultured in 37° C. incubator supplied with 80% humidity and 5% CO2. MDA-MB-468 cells were maintained in Leibovitz's L-15 media supplied with 10% heat-inactivated FBS and cultured in 37° C. incubator supplied with 80% humidity.

### Antibody Expression Constructs

Preliminary anti-CD3 variants were generated in bivalent monospecific IgG1 TM (L234F/L235E/P331S) format for affinity and cross-reactivity assessments. DNA encoding Vκ and VH of prospective anti-CD3 variants were obtained from Integrated DNA Technologies (IDT, Coralville, IA) and assembled into appropriately digested pOE-IgG1 TM (κLC) using NEBuilder HiFi DNA Assembly Master Mix (New England BioLabs, cat no E2621).

For production of monovalent bispecific antibodies, we used the DuetMab platform (Mazor et al., 2015). EGFR-CD3 DuetMab expression constructs encoding the variable domains of anti-EGFR GA201 (Gerdes et al., 2013) and variable domains from the generated CD3 sequences. Because both anti-EGFR and anti-CD3 were derived from κ-containing parental antibodies, pDuetLight vectors were constructed that encoded the anti-EGFR GA201 VL with the κ constant domain and wild-type cysteine and the anti-CD3 VL with the λ constant domain with S121C. In addition, pDuetHeavy vectors were constructed that encoded the anti-EGFR GA201 VH and anti-CD3 VH variants. These pDuetHeavy vectors encoded IgG1 TM (L234F/L235E/P331S) to abolish Fc-mediated effector function. Only sequence-verified expression constructs were used in transient transfections for production of antibodies.

### Expression and Purification of Antibodies

Preliminary anti-CD3 variants were produced from transient transfection of HEK293F cells using 293Fectin™ (Life Technologies) in serum-free FreeStyle293™ media according to the manufacturer's protocol. HEK293F cell culture supernatants that contained antibodies were harvested 6 days after transfection, filtered through a 0.22 μm sterile filter and the concentrations of antibodies were measured by protein A biosensors on an Octet384 instrument (ForteBio) according to the manufacturer's protocol. Antibodies were purified using MabSelect SuRe resin (GE Healthcare), eluted with Pierce IgG Elution Buffer, pH 2.0, and buffer-exchanged into phosphate-buffered saline (PBS), pH 7.2 (Life Technologies). Antibody purity was assessed by HP-SEC and commonly yielded >95% monomer.

DuetMabs were produced by transient co-transfection of CHO-K1 cells with pDuetHeavy- and pDuetLight-based expression vectors using lipofectamine and proprietary media. CHO-K1 cell culture supernatants were harvested at 10 or 12 days post-transfection, filtered and antibodies in culture supernatants were quantified. DuetMabs were initially purified by MabSelect SuRe affinity chromatography and buffer exchanged into PBS, pH 7.2. DuetMabs were then assessed for light chain mispairing by Bioanalyzer A analysis (Agilent) and when required, further purified by LambdaFabSelect (Cytiva) affinity chromatography according to the manufacturer's protocol. After DuetMabs had been buffer-exchanged into PBS, pH 7.2, DuetMabs were loaded on a Superdex 200 16/600 column and fractions containing monomers were collected. DuetMabs were then assessed for purity and light chain mispairing by HP-SEC and Bioanalyzer A, respectively. All DuetMabs exhibited >98% monomer, >95% correct light chain pairing and <0.5 mg/Eu endotoxin.

### HP-SEC

Antibodies were analyzed using HP-SEC to determine purity, including levels of aggregate, monomer, and fragment. Samples (100 μg in PBS) were injected on an Agilent 1200 series high-performance liquid chromatography (HPLC) instrument and separated using a TSKgel G3000SWxl size-exclusion column (Tosoh Bioscience #08541). The mobile phase was 100 mM sodium phosphate (pH 6.8), and sample flow rate was 1 mL/min. Proteins were detected using absorbance at 280 nm.

### Flow Cytometry Measurement

Flow Cytometry was used to determine specific cell surface antigen binding. The human leukemia T cell lines HPB-ALL, Jurkat, Jurkat TCR KO, and cynomolgus T cell line HSC-F were used to assess human CD3 and cynomolgus CD3 binding, respectively. Briefly, 1×10⁵ cells were suspended in FACS buffer (1×PBS, pH 7.2 supplemented with 2% heat-inactivated FBS, 0.1% sodium azide and 2 mM EDTA) and placed into each well of a round-bottom 96-well plate. Antibodies were diluted to various concentrations using FACS buffer and added to the cells. Antibodies and cells were washed twice with FACS buffer after incubation at 4° C. for 30 min. Cell surface antigen binding of antibodies was detected with Alexa Fluor 647-conjugated goat F(ab′)₂ fragment specifically against human IgG Fcγ fragment. Data was collected using a BD FACSymphony A3 Cell Analyzer (BD Biosciences). Data was analyzed with FlowJo v10.6.1 and plotted using GraphPad Prism v9.0.0.

### Analysis of EGF Receptor Density

Quantitative analysis of receptor density on the tumor cell lines was performed by flow cytometry on a BD FACSymphony™ A3 Cell Analyzer (BD Biosciences). Briefly, anti-EGFR human IgG1 was first labelled with Alexa Fluor 647 using protein labeling kit (Invitrogen) according to manufacturer's instruction. Antibody concentration and fluorochrome to protein (F:P) ratio were determined by a ND-1000 spectrophotometer (NanoDrop). Detached tumor cells were washed and resuspended with ice-cold FACS buffer (1×PBS pH 7.2, 2% heat-inactivated FBS, 2 mM EDTA and 0.1% sodium azide). 2×106 cells were then incubated with Alexa Fluor 647 conjugated antibodies at saturating concentrations (≥20 μg/mL) for 1 h at 4° C. After washing with FACS buffer, cells were fixed in ice-cold 1.8% paraformaldehyde (PFA) and detection of bound antibodies was performed on BD FACSymphony A3 Cell Analyzer using BD FACSDiva™ software. Results were analyzed using the FlowJo analysis software (Tree Star). For quantitation of EGFR receptor density on cells, Quantum Alexa Fluor 647 MESF (Molecules of Equivalent Soluble Fluorochrome) beads (Bangs Laboratories) were analyzed on the flow cytometer using similar settings to establish a standard curve. Using the QuickCal program (Bangs Laboratories) the calculated MESF was then divided by the antibody F:P ratio to give a corrected Antibody Binding Capacity (ABC).

### Cytotoxicity and T Cell Activation

T cell-mediated cytotoxicity and T cell activation were assessed by flow cytometry measurement. Cell-specific growth media (RPMI 1640+10% FBS for NCI H358 or L-15+10% FBS for MDA-MB-468) supplemented with 50 μM β-mercaptoethanol was used as assay media. A total of 1×10⁴ EGFR+ target cells (NCI H358 or MDA-MB-468) were stained with CellTrace Violet (ThermoFisher Scientific) and seeded in each well of a tissue culture-treated round-bottom 96-well plate. Effector cells, PBMCs from a healthy donor, were added at E:T ratio of 10:1 and antibodies were added at various concentrations. After one day of incubation at 37° C. in humidified incubator (5% CO2 for NCI H358 or 0% CO2 for MDA-MB-468), all cells from the cytotoxicity assay were harvested, stained and analyzed by flow cytometry using a Symphony A3 (BD). EGFR+ target cells were identified by CellTrace Violet staining and T cells were identified by CD4+ or CD8+ staining. T cell activation was determined by CD69+ and CD25+. Data was analyzed using FlowJo v10.6.1. Cell cytotoxicity was normalized to the minimum cytotoxicity value without antibody treatment and plotted using GraphPad Prism v9.0.0.

### Cytokine Release Assay

Cytokines released into supernatants from cytotoxicity assays were evaluated using ProcartaPlex Human, NHP, and Canine Mix & Match Panels Luminex Kit (ThermoFisher Scientific) according to the manufacturer's protocol. The plates were read on Bio-Plex 3D Suspension Array System (Bio-Rad) using Luminex xPONENT software. Data was analyzed and plotted using GraphPad Prism v9.0.0.

### Assessment of T Cell Activation in the Absence of EGFR+ Cells

To assess the level of T cell activation induced by anti-CD3 variants in the absence of EGFR+ cells, we used a plate-based PBMC incubation assay. Flat-bottom 96-well plates were coated overnight at 4° C. with varied concentrations of T cell engager (50, 5.0, 0.5 nM) in 50 μL. Plates were then washed twice with PBS, pH 7.2 before addition of 150,000 PBMCs in 200 μL AIM V media. PBMCs were incubated for 48 hours at 37° C. PBMCs were then transferred to a 96-well round bottom plate, centrifuged, and cell pellets were washed twice in PBS and stained for CD69 and CD25 expression using an anti-CD2, -CD3, -CD4, -CD8, -CD25, -CD69 panel of antibodies by flow cytometry.

### Kinetics Measurements

The binding kinetics of anti-CD3 variant-based DuetMabs were determined using Octet analysis. Biotinylated recombinant human CD380 (Acro Biosystems, CDD-H82W6) was immobilized onto streptavidin sensors at a concentration of 1 μg/mL followed by 2-fold dilutions of anti-CD3 variant DuetMabs. For the parental anti-CD3 DuetMab, concentrations from 300 to 4.7 nM were evaluated. For anti-CD3 variant DuetMabs, concentrations from either 5 μM to 78.1 nM or 8 μM to 125 nM were used. Kinetics were analyzed using a 1:1 antibody:antigen binding model.

### AC-SINS

Affinity-capture self-interaction nanoparticle spectroscopy (AC-SINS) was used to assess antibody self-association and was performed as described elsewhere (Dippel et al., 2023). Briefly, 5 μL of nanoparticles were mixed with 45 μL of purified antibody at 50 ug/mL in PBS, pH 7.2 or HSA buffer [20 mM histidine, 120 mM sucrose, 80 mM arginine, pH 6] in a 384-well plate. Nanoparticles were mixed with buffer only (no antibody) as a control. Absorbance was measured on a SPECTROstar Nano UV/vis plate reader from 490 to 700 nm. The wavelength of peak absorbance was calculated in the MARS data analysis software and used to determine the wavelength shift compared to the nanoparticle-only control.

### Differential Scanning Fluorimetry

Differential-scanning fluorimetry (DSF) was used to assess the thermal melting temperatures of DuetMabs. Measurements were performed using a previously established method with minor modifications (Shan et al., 2018). Samples were prepared by combining 20 μL of protein sample at 1 mg/mL in PBS, pH 7.2 with 5 μL of SYPRO Orange dye (Invitrogen S-6651) diluted to 40× in PBS (pH 7.2) in a 96-well PCR plate in duplicate. The plate was sealed and measurements performed in a QuantStudio 7 Flex Real-Time PCR System (Applied Biosystems). Samples were subjected to an initial equilibration step at 25° C. for 2 m, followed by a temperature ramp to 99° C. at 0.05° C./sec increments. The fluorescence emission was monitored using the FAM filter set. The Tm value for each sample was calculated in the Protein Thermal Shift software (Applied Biosystems) using the Boltzmann method.

### Accelerated Stability and Heat Stress Study

For accelerated stability testing, samples were diluted to 1 mg/mL in PBS, pH 7.2 and incubated for 2 weeks at either 4° C. or 45° C. Samples were then analyzed by HP-SEC. The monomer, aggregate, and fragment percentages for each sample were calculated based on curve integration using the HPLC ChemStation software (Agilent). The change in monomer, aggregate, and fragment content was calculated from the difference between each sample incubated at 45° C. versus 4° C.

### Example 2—Construction of Parental Antibody

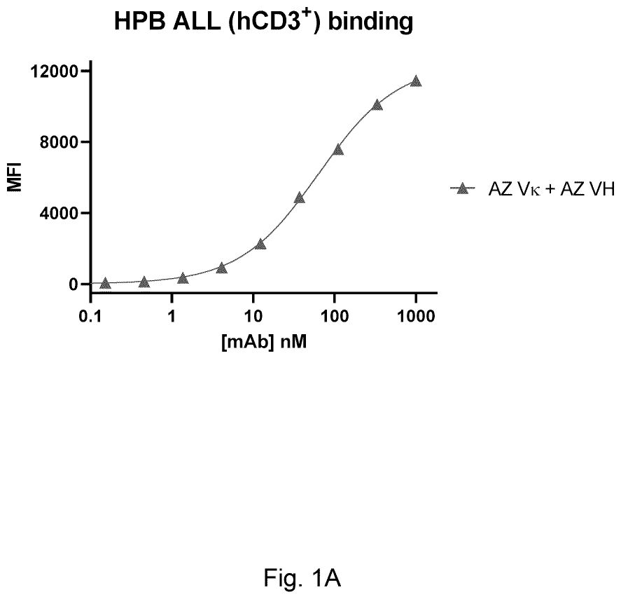

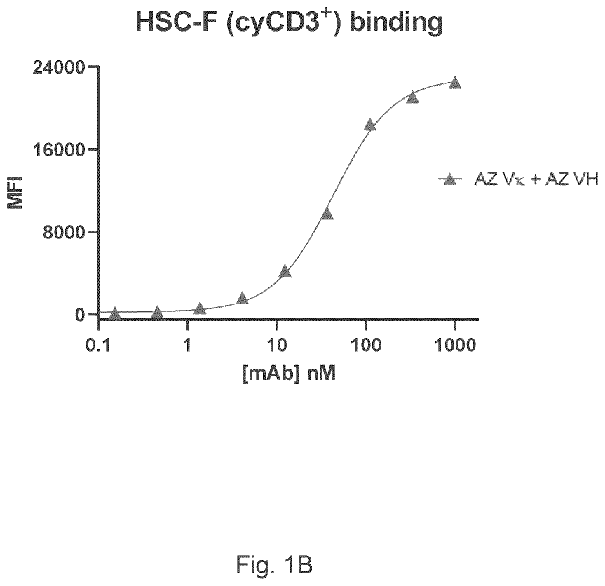

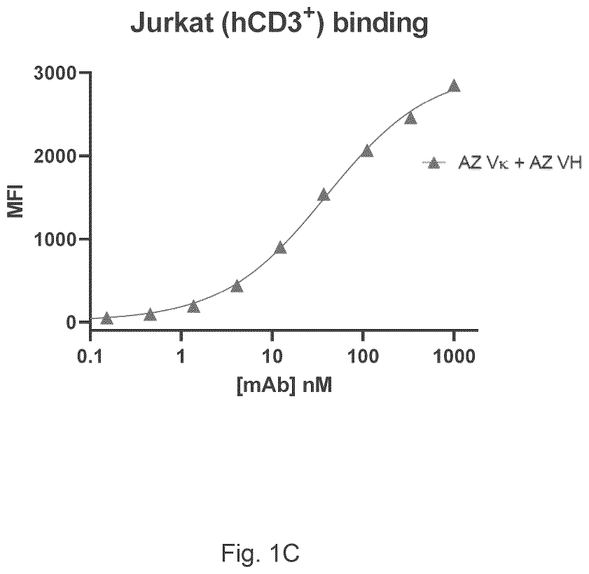

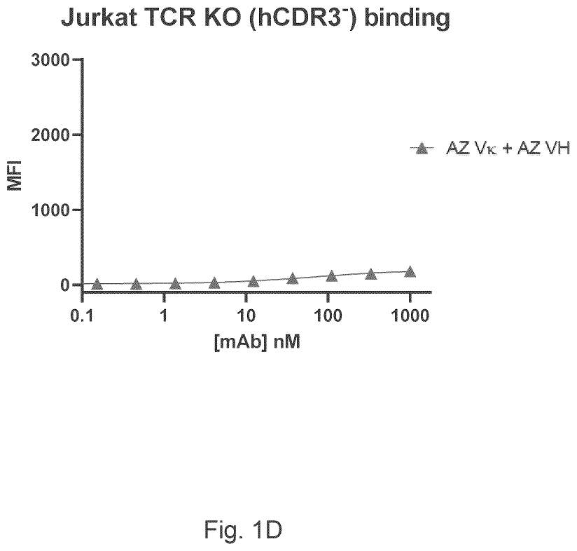

We generated AZ Vκ+AZ VH IgG1-TM, which was evaluated for binding CD3+ cells, including HPB ALL (high human CD3+), Jurkat (human CD3+), Jurkat (TCR KO, human CD3−), and HSC-F (high cynomolgus monkey CD3+) by flow cytometry measurement. Results are summarized in FIG. 1. FIG. 1 shows that AZ Vκ+AZ VH IgG1-TM maintains human and cynomolgus monkey CD3 cross-reactivity of the parental antibody (FIGS. 1A and B), and exhibits selective binding to CD3⁺ cells as demonstrated by a lack of binding to TCR KO Jurkat cells (see FIGS. 1C and D).

### Example 3—Mitigating Liabilities Within CDRs

Potential sequence liabilities pose a risk for large-scale manufacturing. For example, NN and NG deamidation sites were found within CDR L1, a tryptophan oxidation site within CDR H1, and a DK isomerization site and a NS deamidation site within CDR H2. Therefore, initial CDR engineering efforts focused on mitigating these liabilities.

To mitigate the NN and NG deamidation sites within CDR L1, constructs were generated that mutated the NNG sequences to NQA or NTG where the underlined amino acids differ from the parental anti-CD3. In addition, constructs were generated that also mutated the phenylalanine in CDR L2 to alanine or tyrosine, where alanine is present in the human IGKV2D-28*01 germline and tyrosine is a hydrophobic amino acid with similar structure to phenylalanine.

Similarly, to mitigate the tryptophan oxidation site within CDR H1, constructs were generated that mutated the tryptophan to tyrosine. In addition, constructs were generated that mutated threonine-lysine at the FW1-CDR H1 junction and histidine at the end of CDR H1 to serine-asparagine and serine, respectively, which are present in the human IGHV3-15*02 germline. To mitigate potential liabilities within CDR H2, DK was mutated to DR and NS to QS or QT.

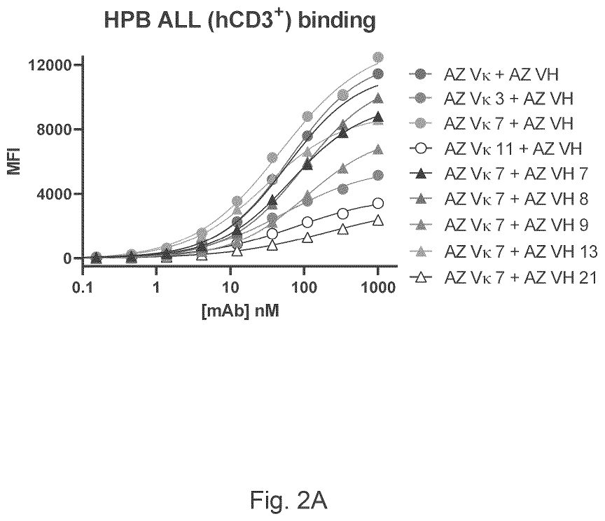

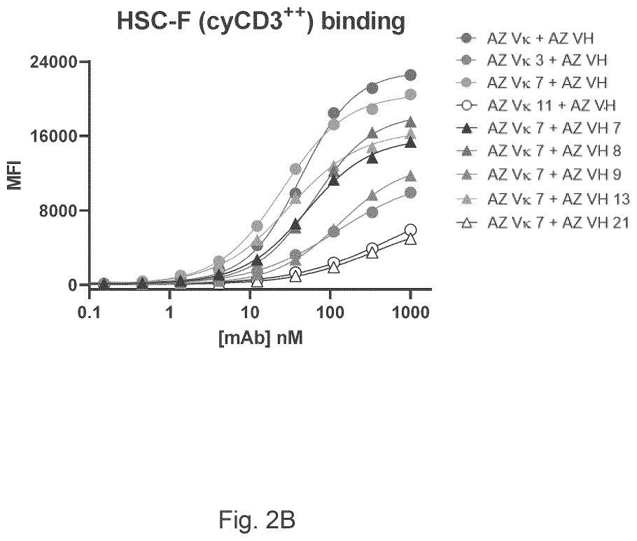

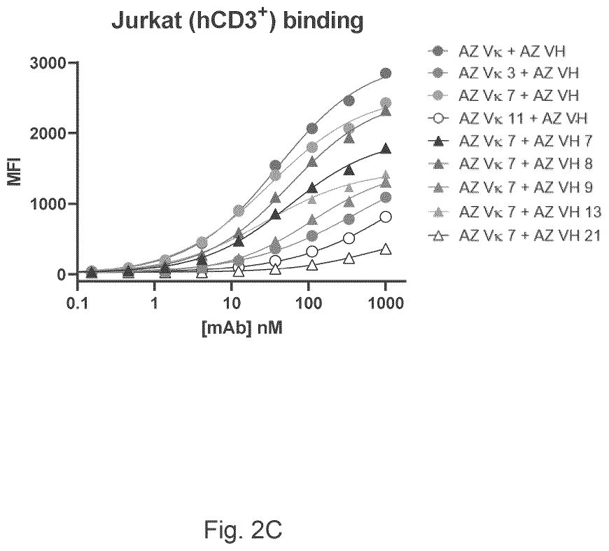

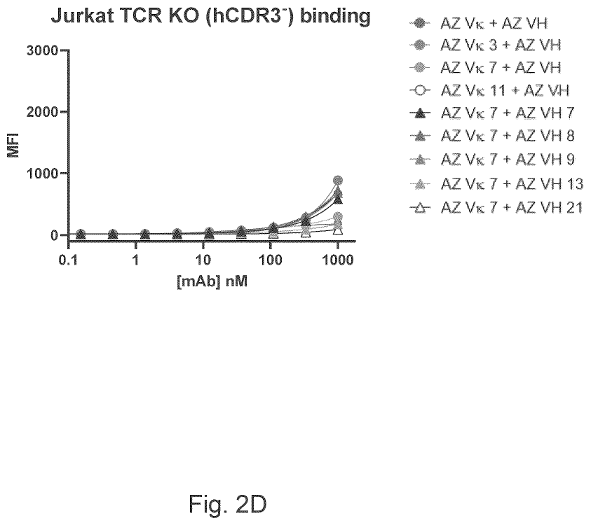

Combinations of the Vκ and VH variants were then generated and binding to HPB ALL (high human CD3+), Jurkat (human CD3+), Jurkat (TCR KO, human CD3−), HSC-F (high cynomolgus CD3+) was evaluated by flow cytometry measurement. Results are summarized in FIG. 2, which shows that variants exhibit a spectrum of affinities for human and cynomolgus monkey CD3+ cells; and importantly, variants maintain binding to cynomolgus CD3+ cells.

Leads that maintained binding to CD3+ cells were prioritized and additionally identified variants that specifically modified CDR L3 and CDR H3 to impart desirable cytotoxicity, T cell activation, and cytokine release profiles.

### Example 4—Alanine Scanning of CDR L3 and CDR H3

To identify the residues within CDR L3 and CDR H3 that were directly and indirectly involved in the anti-CD3 paratope, alanine-scanning mutagenesis of these regions was performed and binding of variants to CD3+ cells was evaluated by flow cytometry. The results were interpreted as follows: (1) variants that abolished binding to CD3+ cells contained mutations that disrupted the anti-CD3 paratope; (2) variants that exhibited diminished binding contained mutations that merely perturbed the paratope; (3) variants that exhibited unaffected binding contained mutations that were not involved in the paratope.

Results show that Vκ variants that contained G91A or F96A (Kabat numbering) abolished CD3+ cell binding, variants that contained G89A, Q90A, T92A showed modestly diminished binding, and variants that contained Q93A, Y94A, P95A or T97A did not affect binding. Similarly, VH variants that contained Y97A and A98L abolished CD3+ cell binding, whereas variants that contained R93A, G94A, V95A, Y96A, P100aA, F100bA or Y102A exhibited modestly diminished binding, and variants that contained L99A, S100A, or D101A showed unaffected binding profiles.

### Example 5—Functional Characterization of Anti-CD3 Variants in a Relevant Bispecific Format

Binding to CD3+ cells is only a pre-requisite for T cell engager function and the aim is to optimize the functional activity of the CD3-binding arm within the context of a relevant bispecific T cell engager. Therefore, monovalent bispecific antibodies were constructed based on the well-established DuetMab platform (Mazor et al., 2015; Wang et al., 2020; Dovedi et al., 2021) to determine the functional effects of anti-CD3 variants on affinity, cytotoxicity, T cell activation, and cytokine release. These DuetMabs were composed of anti-EGFR and anti-CD3 variant binding arms.

It is important to note that functional characterization of anti-CD3 variants was necessary to identify the variants with the appropriate affinity, cytotoxicity, T cell activation and cytokine release. Ultimately, three light chain variants (AZ Vκ 17, AZ Vκ 26, and AZ Vκ 29) and nine heavy chain variants (AZ VH R75, AZ VH R79, AZ VH R82, AZ VH SN75, AZ VH SN79, AZ VH SN82, AZ VH E75, AZ VH E79, and AZ VH E82) were chosen as the composition of anti-CD3 variants for further investigation. The twenty-seven anti-CD3 variants that derived from these Vκ and VH combinations were extensively characterized.

EGFR-CD3 variant DuetMabs were characterized for CD3+ cellular binding affinity and functionally profiled for cytotoxicity, T cell activation, and cytokine release upon exposure to EGFR+ cells. To enable direct comparisons, all functional assays were completed with a single batch of frozen peripheral blood mononuclear cells (PBMCs) to avoid the inherent donor-to-donor heterogeneity of freshly isolated PBMCs. The batch of PBMCs selected reflects the median reactivity of PBMCs surveyed. In addition, we assessed functional activity for all variants using low EGFR+ NCI H358 cells (3.1×104±receptors/cell) (Mazor et al., 2017) and select variants using high EGFR+ MDA-MB-468 (1.1×106±2.8×104 receptors/cell).

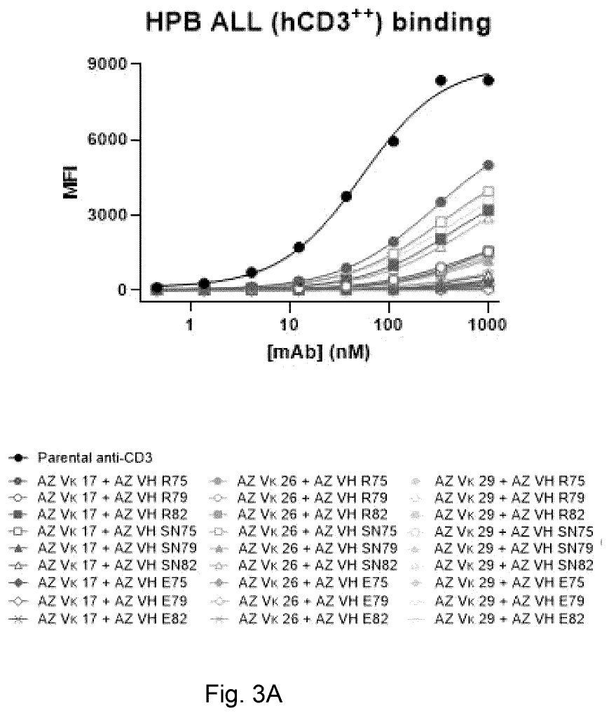

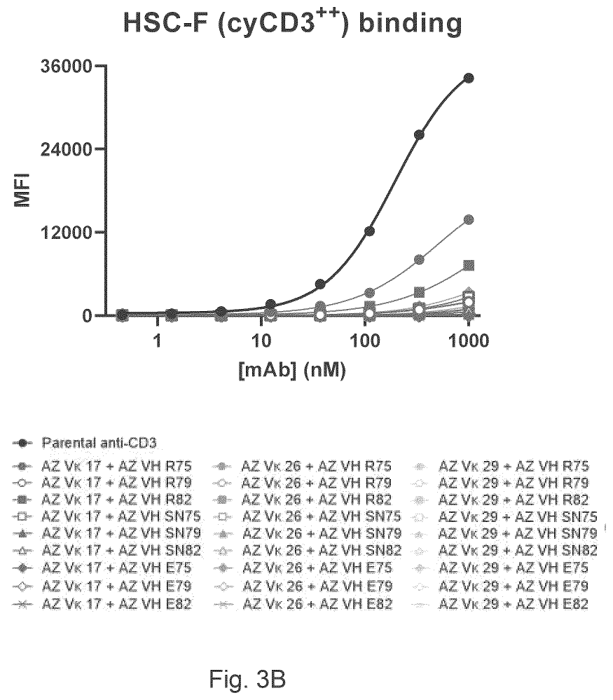

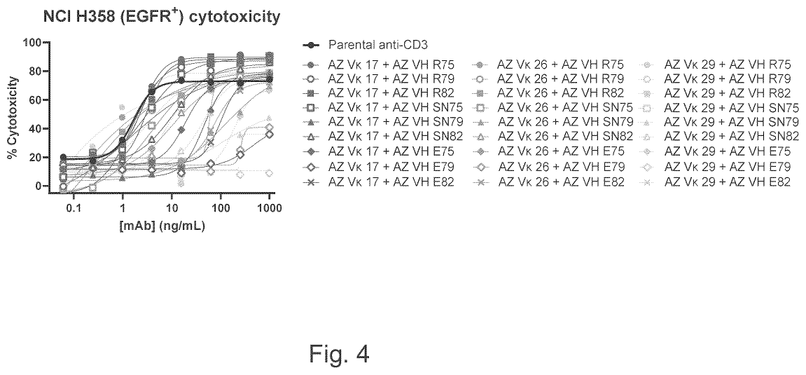

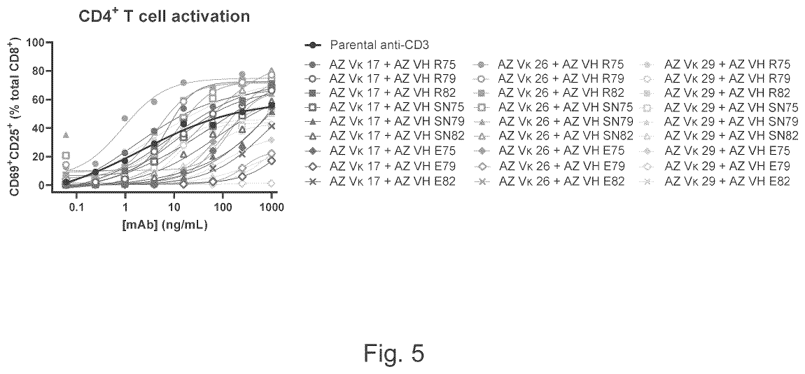

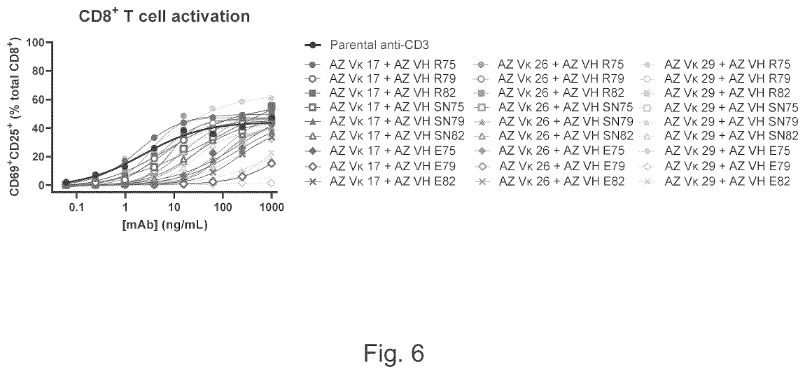

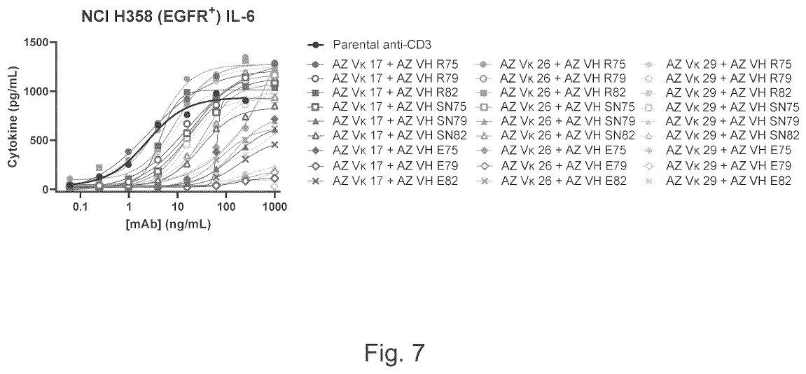

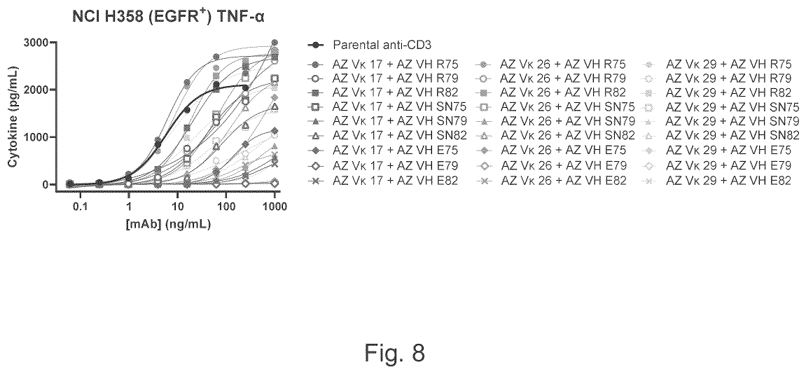

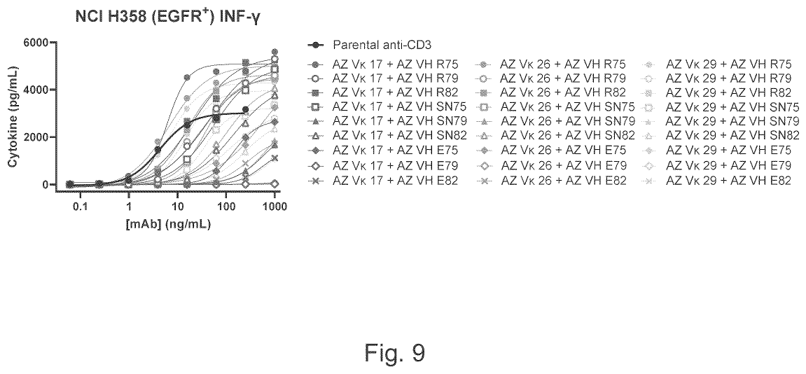

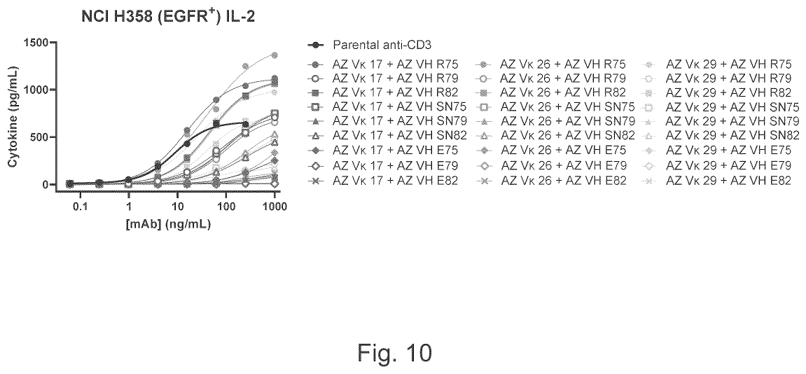

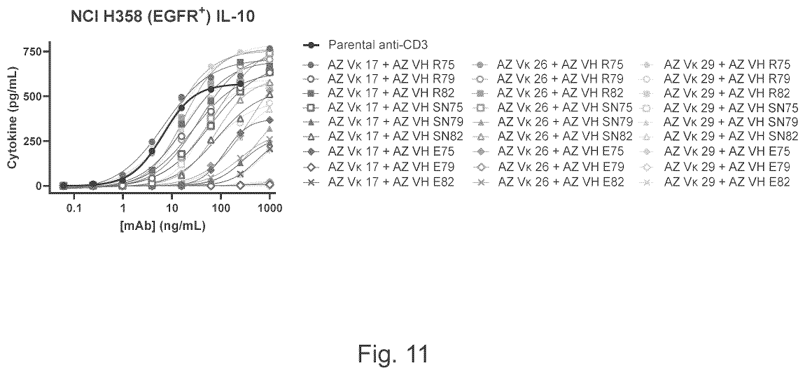

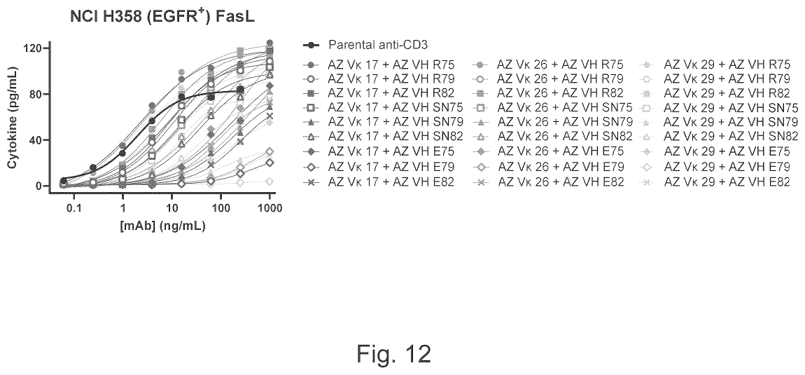

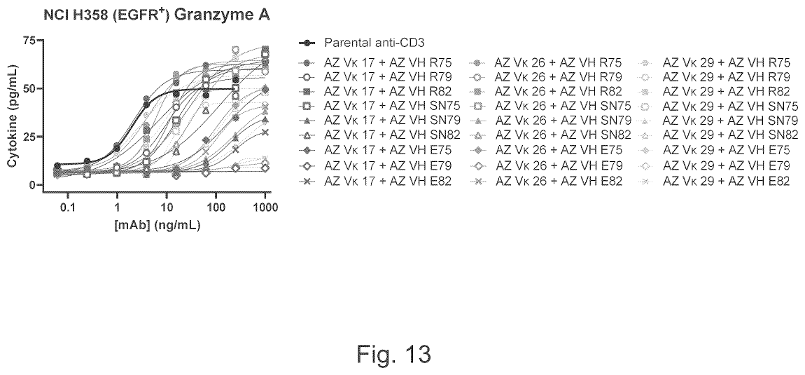

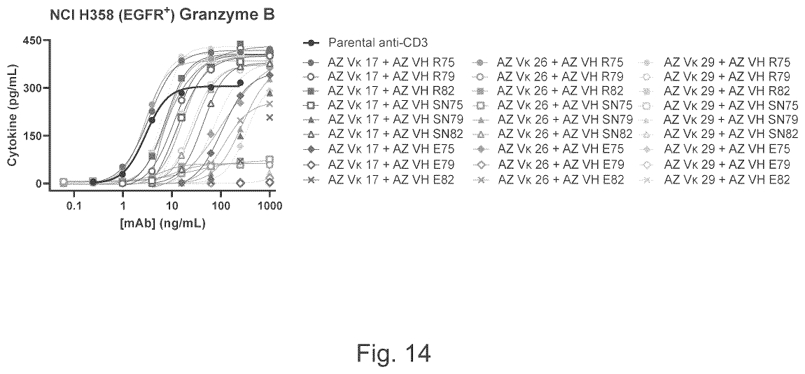

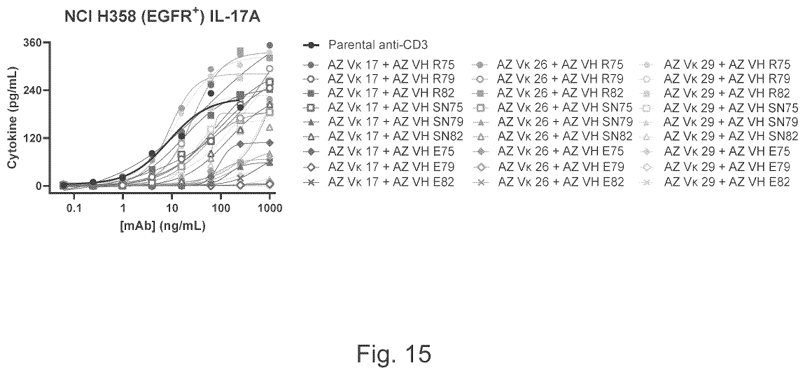

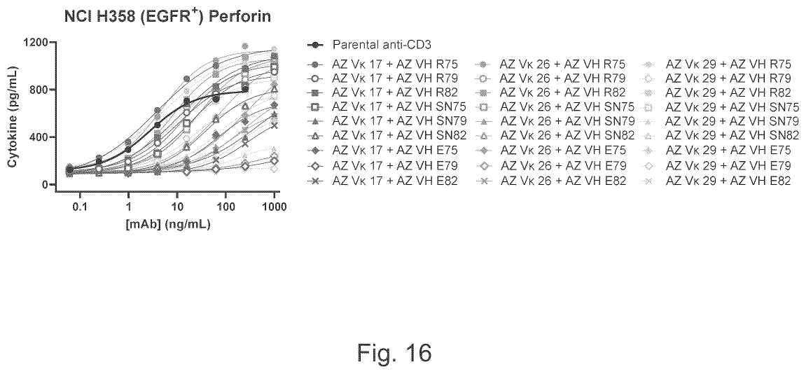

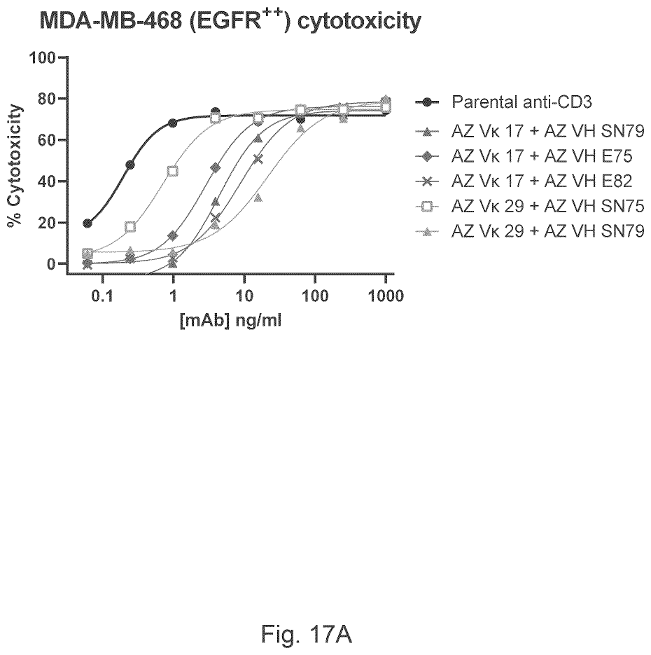

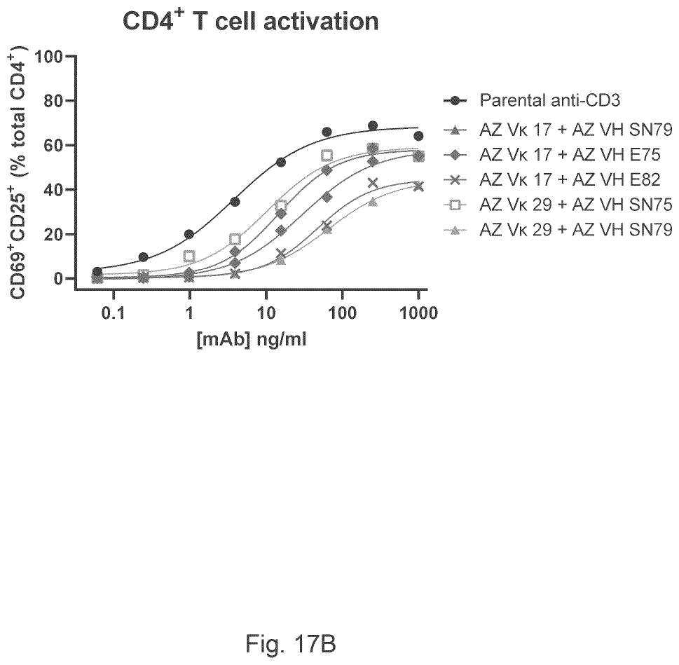

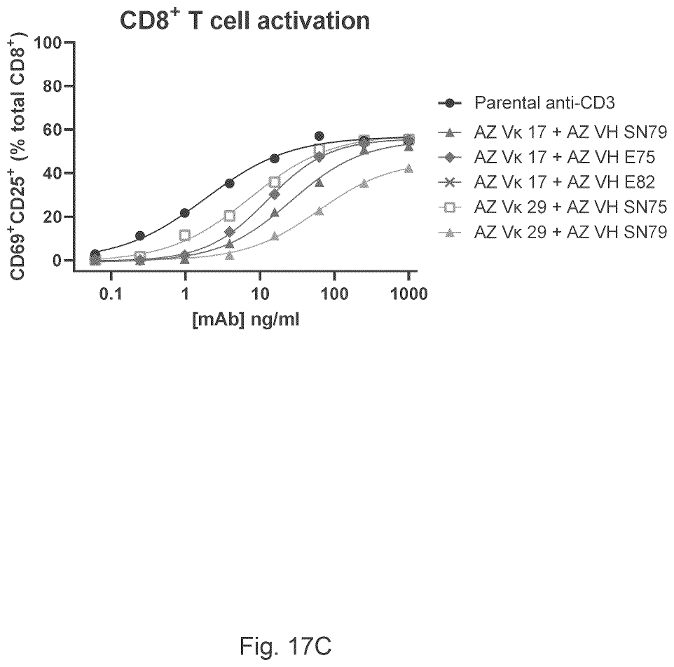

Results are shown in FIGS. 3 to 17. DuetMabs containing anti-CD3 variants exhibit variable affinity for CD3+ cells, cytotoxicity of EGFR+ cells, T cell activation and cytokine release profiles. Furthermore, the functional responses elicited by EGFR-CD3 variant DuetMabs were EGFR-dependent. For example, DuetMabs that exhibited very little activity upon exposure to the low EGFR+ NCI H358 exhibited enhanced activity on the high EGFR+ MDA-MB-468. Therefore, the optimal anti-CD3 variant composition of T cell engagers can be selected based on the desired TAA density or TAA-CD3 format (1+1, 2+1, 1+1+1, etc.).

### Example 6—Assessment of T Cell Activation in the Absence of Target Cells

The anti-CD3 variant-based T cell engagers were evaluated for performance in the presence of EGFR+ target cells and T cells. However, it was also important to understand the behavior of anti-CD3 variant-containing T cell engagers in the absence of EGFR+ cells as premature activation of T cells can lead to undesirable toxicities and cytokine release syndrome (CRS) in vivo.

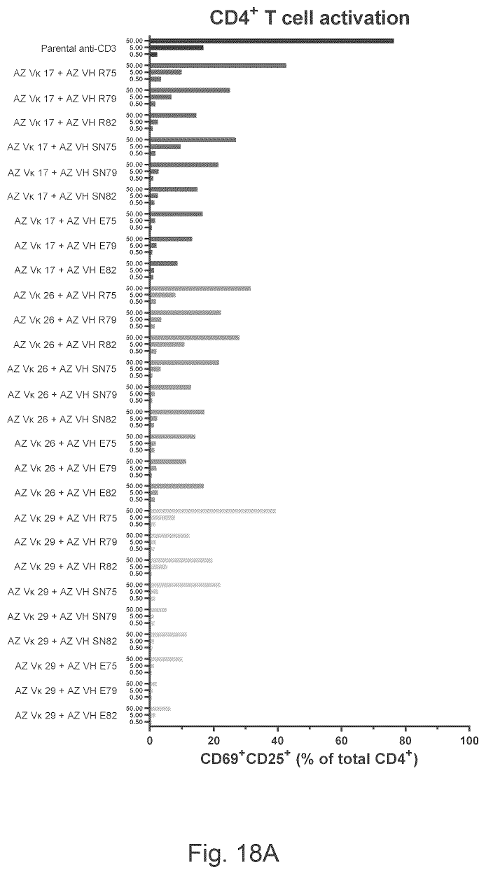

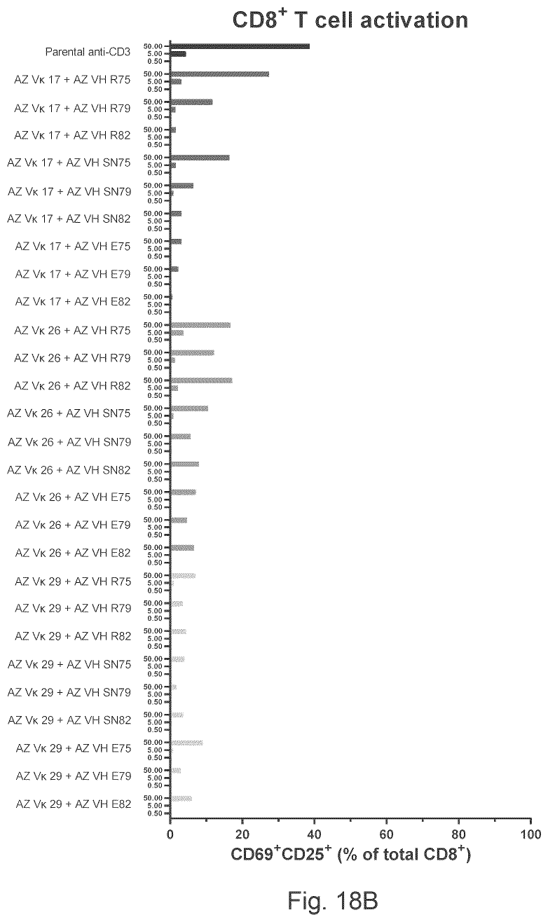

In order to assess the level of T cell activation induced by our anti-CD3 variant-containing T cell engagers in the absence of EGFR+ cells, we used a plate-based PBMC incubation assay. In this assay, PBMCs were incubated in 96-well plates that had been pre-coated with varied concentrations of candidate T cell engagers (50, 5.0, 0.5 nM). After exposure for 48 hours, the level of T cell activation of CD4+ and CD8+ T cells were characterized by flow cytometry using CD25 and CD69 markers. Results are summarized in FIG. 18 and indicate that all anti-CD3 variant-containing T cell engagers exhibit reduced T cell activation compared with the anti-CD3-based control.

### Example 7—Kinetic Measurement

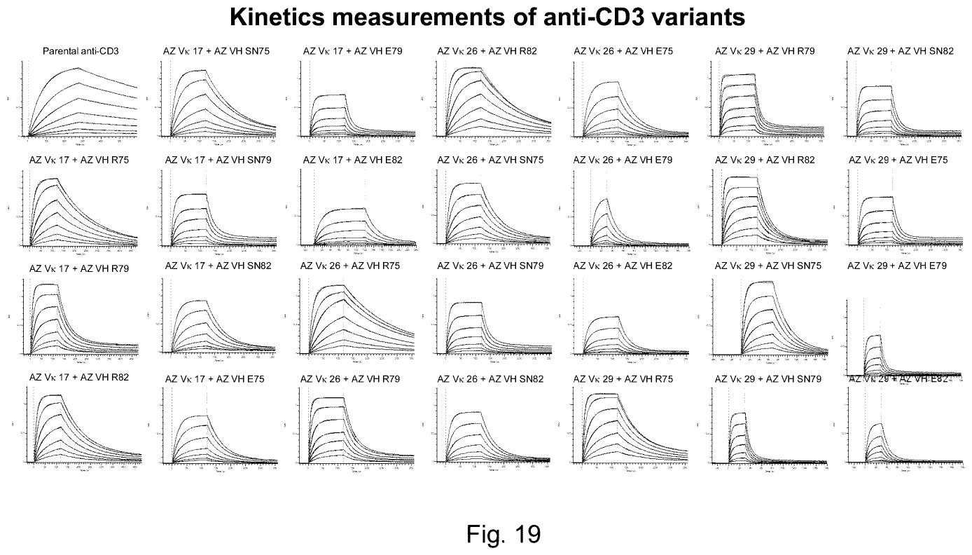

The binding kinetics of anti-CD3 variant antibodies were determined using Octet analysis. Results are shown in FIG. 19 and Table 1. Variants exhibited between 10-to 100-fold reduction in human CD3 affinity.

TABLE 1
Kinetics measurements to the soluble form of CD3 were obtained using an Octet384 instrument. The dissociation
constants, KD, were calculated as a ratio of k_{off}/kₒₙ from a non-linear fit of the data.

| | K_{D} | K_{D} | ka | ka | kdis | kdis | Full | Full |
|---|---|---|---|---|---|---|---|---|
| αCD3 variant | (M) | Error | (1/Ms) | Error | (1/s) | Error | X\hat{ }2 | R\hat{ }2 |
| Parental anti-CD3 | 2.43E−08 | 1.89E−11 | 4.33E+04 | 2.39E+01 | 1.05E−03 | 5.78E−07 | 0.3325 | 0.9999 |
| AZ Vk 17 + AZ VH R75 | 2.66E−07 | 9.61E−10 | 2.42E+04 | 4.84E+01 | 6.44E−03 | 1.93E−05 | 0.4807 | 0.9994 |
| AZ Vk 17 + AZ VH R79 | 7.34E−07 | 1.60E−09 | 2.46E+04 | 4.51E+01 | 1.80E−02 | 2.13E−05 | 0.3476 | 0.9996 |
| AZ Vk 17 + AZ VH R82 | 5.50E−07 | 1.52E−09 | 1.69E+04 | 3.12E+01 | 9.28E−03 | 1.90E−05 | 0.4113 | 0.9994 |
| AZ VK 17 + AZ VH SN75 | 8.53E−07 | 1.36E−09 | 1.08E+04 | 1.18E+01 | 9.23E−03 | 1.07E−05 | 0.1095 | 0.9998 |
| AZ VK 17 + AZ VH SN79 | 2.05E−06 | 6.69E−09 | 1.56E+04 | 4.25E+01 | 3.20E−02 | 5.73E−05 | 0.1498 | 0.9996 |
| AZ VK 17 + AZ VH SN82 | 1.64E−06 | 4.56E−09 | 9.10E+03 | 1.73E+01 | 1.49E−02 | 3.03E−05 | 0.0948 | 0.9998 |
| AZ Vk 17 + AZ VH E75 | 2.79E−06 | 9.34E−09 | 7.10E+03 | 1.94E+01 | 1.98E−02 | 3.85E−05 | 0.1017 | 0.9997 |
| AZ VK 17 + AZ VH E79 | 5.62E−06 | 4.17E−08 | 8.85E+03 | 5.51E+01 | 4.97E−02 | 2.00E−04 | 0.1967 | 0.9991 |
| AZ VK 17 + AZ VH E82 | 4.61E−06 | 2.34E−08 | 7.14E+03 | 3.28E+01 | 3.30E−02 | 7.14E−05 | 0.0948 | 0.9995 |
| AZ Vk 26 + AZ VH R75 | 2.17E−07 | 7.79E−10 | 2.79E+04 | 5.32E+01 | 6.05E−03 | 1.84E−05 | 0.5034 | 0.9994 |
| AZ Vk 26 + AZ VH R79 | 7.12E−07 | 2.07E−09 | 3.27E+04 | 8.39E+01 | 2.33E−02 | 3.20E−05 | 0.5156 | 0.9992 |
| AZ Vk 26 + AZ VH R82 | 4.11E−07 | 1.03E−09 | 1.73E+04 | 2.54E+01 | 7.11E−03 | 1.44E−05 | 0.3284 | 0.9996 |
| AZ Vk 26 + AZ VH SN75 | 9.49E−07 | 2.84E−09 | 1.63E+04 | 3.09E+01 | 1.55E−02 | 3.59E−05 | 0.1873 | 0.9997 |
| AZ Vk 26 + AZ VH SN79 | 1.93E−06 | 6.31E−09 | 1.87E+04 | 5.19E+01 | 3.61E−02 | 6.26E−05 | 0.1571 | 0.9996 |
| AZ Vk 26 + AZ VH SN82 | 1.71E−06 | 4.44E−09 | 1.24E+04 | 2.43E+01 | 2.12E−02 | 3.63E−05 | 0.0922 | 0.9998 |
| AZ Vk 26 + AZ VH E75 | 2.28E−06 | 4.23E−09 | 7.54E+03 | 1.05E+01 | 1.72E−02 | 2.10E−05 | 0.0403 | 0.9999 |
| AZ Vk 26 + AZ VH E79 | 4.66E−06 | 5.20E−08 | 1.10E+04 | 1.06E+02 | 5.11E−02 | 2.86E−04 | 0.0884 | 0.9988 |
| AZ Vk 26 + AZ VH E82 | 3.92E−06 | 1.70E−08 | 7.33E+03 | 2.79E+01 | 2.87E−02 | 5.88E−05 | 0.0846 | 0.9995 |
| AZ Vk 29 + AZ VH R75 | 2.24E−07 | 4.97E−10 | 5.33E+04 | 8.83E+01 | 1.19E−02 | 1.76E−05 | 0.3829 | 0.9995 |
| AZ Vk 29 + AZ VH R79 | 8.78E−07 | 5.08E−09 | 5.55E+04 | 2.82E+02 | 4.87E−02 | 1.35E−04 | 0.8738 | 0.9984 |
| AZ Vk 29 + AZ VH R82 | 5.95E−07 | 1.46E−09 | 3.88E+04 | 8.40E+01 | 2.31E−02 | 2.64E−05 | 0.4141 | 0.9994 |
| AZ VK 29 + AZ VH SN75 | 1.01E−06 | 3.03E−09 | 2.79E+04 | 7.63E+01 | 2.82E−02 | 3.44E−05 | 0.3159 | 0.9994 |
| AZ VK 29 + AZ VH SN79 | 2.67E−06 | 3.17E−08 | 4.28E+04 | 4.64E+02 | 1.14E−01 | 5.61E−04 | 0.2298 | 0.9976 |
| AZ Vk 29 + AZ VH SN82 | 1.86E−06 | 5.97E−09 | 2.27E+04 | 6.33E+01 | 4.22E−02 | 6.76E−05 | 0.1423 | 0.9996 |
| AZ VK 29 + AZ VH E75 | 2.22E−06 | 9.28E−09 | 1.82E+04 | 6.59E+01 | 4.04E−02 | 8.41E−05 | 0.2000 | 0.9994 |
| AZ VK 29 + AZ VH E79 | 4.58E−06 | 6.12E−08 | 2.58E+04 | 3.20E+02 | 1.18E−01 | 5.91E−04 | 0.1327 | 0.9978 |
| AZ Vk 29 + AZ VH E82 | 5.00E−06 | 6.23E−08 | 1.51E+04 | 1.70E+02 | 7.55E−02 | 4.02E−04 | 0.0926 | 0.9982 |

### Example 8—Developability

Poor behaving antibodies have been correlated with poor clinical success and as a result, it has become increasingly common to identify and mitigate risks prior to lead selection. To this end, anti-CD3 variants were further characterized for expression titer, non-specific binding, reversible self-association, thermal stability, and aggregation and fragmentation propensity post-thermal stress. Results of these assessments are summarized in Table 2.

TABLE 2
Summary of the developability profiles of EGFR-CD3 variant antibodies. Prior to biochemical, biophysical
and biological characterization, antibodies were purified by protein A affinity chromatography followed
by light chain affinity chromatography, and then subjected to preparative SEC for aggregate removal.

| | | | | | | | | Accelerated stability (45° C., 14 d) | | | |
|---|---|---|---|---|---|---|---|---|---|---|---|
| | | | HEK | | | | | | | | ΔRT |
| | BVP ELISA | | binding | | | DSF | | Monomer | Aggregate | Fragment | from |
| αCD3 | BVP | BSA/Plate | Cell | AC−SINS | | Tₒₙₛₑₜ | Tₘ | loss | increase | increase | NIP228 |
| variant | score | binding | binding? | PBS (nm) | HSA (nm) | (° C.) | (° C.) | (%) | (%) | (%) | (min) |
| Parental anti- | 6.42 | 0.10 | N | 1.75 | 1.25 | 49.57 | 61.22 | −2.38 | 0.49 | 1.88 | 0.07 |
| CD3 | | | | | | | | | | | |
| AZ VK 17 + AZ | 3.85 | 0.06 | N | 1.75 | 2.25 | 52.02 | 61.18 | −3.71 | 0.59 | 3.12 | 0.07 |
| VH R75 | | | | | | | | | | | |
| AZ Vk 17 + AZ | 2.87 | 0.04 | N | 1.75 | 0.25 | 49.74 | 60.81 | −2.40 | 1.16 | 1.25 | 0.09 |
| VH R79 | | | | | | | | | | | |
| AZ Vk 17 + AZ | 2.87 | 0.05 | N | 1.75 | 0.75 | 50.45 | 61.2 | −3.18 | 0.83 | 2.35 | 0.06 |
| VH R82 | | | | | | | | | | | |
| AZ VK 17 + AZ | 2.06 | 0.03 | N | 1.25 | 0.75 | 50.27 | 61.57 | −1.67 | 0.00 | 1.67 | 0.06 |
| VH SN75 | | | | | | | | | | | |
| AZ VK 17 + AZ | 2.75 | 0.04 | N | 1.75 | 0.75 | 50.09 | 61.09 | −1.72 | 0.35 | 1.37 | 0.08 |
| VH SN79 | | | | | | | | | | | |
| AZ Vk 17 + AZ | 1.89 | 0.03 | N | 1.75 | 0.75 | 50.97 | 61.47 | −1.40 | 0.00 | 1.40 | 0.06 |
| VH SN82 | | | | | | | | | | | |
| AZ VK 17 + AZ | 1.97 | 0.03 | N | 1.75 | 0.25 | 50.8 | 60.69 | −1.89 | 0.55 | 1.34 | 0.05 |
| VH E75 | | | | | | | | | | | |
| AZ VK 17 + AZ | 2.20 | 0.03 | N | 1.25 | 0.25 | 49.57 | 60.87 | −2.14 | 0.59 | 1.55 | 0.07 |
| VH E79 | | | | | | | | | | | |
| AZ VK 17 + AZ | 2.42 | 0.04 | N | 1.25 | 0.75 | 50.09 | 61.41 | −1.67 | 0.23 | 1.43 | 0.05 |
| VH E82 | | | | | | | | | | | |
| AZ Vk 26 + AZ | 2.12 | 0.04 | N | 1.75 | 0.75 | 51.15 | 60.58 | −2.15 | 0.79 | 1.37 | 0.06 |
| VH R75 | | | | | | | | | | | |
| AZ Vk 26 + AZ | 2.17 | 0.05 | N | 1.75 | 1.25 | 49.92 | 60.1 | −2.47 | 0.97 | 1.50 | 0.08 |
| VH R79 | | | | | | | | | | | |
| AZ Vk 26 + AZ | 2.57 | 0.05 | N | 1.75 | 0.75 | 49.74 | 60.53 | −2.33 | 0.70 | 1.64 | 0.06 |
| VH R82 | | | | | | | | | | | |
| AZ Vk 26 + AZ | 1.68 | 0.03 | N | 1.25 | 0.75 | 50.79 | 61.2 | −1.81 | 0.24 | 1.56 | 0.06 |
| VH SN75 | | | | | | | | | | | |
| AZ VK 26 + AZ | 2.26 | 0.04 | N | 1.75 | 0.75 | 49.92 | 60.32 | −1.94 | 0.46 | 1.48 | 0.08 |
| VH SN79 | | | | | | | | | | | |
| AZ Vk 26 + AZ | 2.27 | 0.04 | N | 1.75 | 1.25 | 50.27 | 60.72 | −1.31 | 0.00 | 1.31 | 0.06 |
| VH SN82 | | | | | | | | | | | |
| AZ Vk 26 + AZ | 1.64 | 0.03 | N | 1.75 | 0.75 | 50.45 | 60.3 | −3.40 | 1.20 | 2.20 | 0.05 |
| VH E75 | | | | | | | | | | | |
| AZ VK 26 + AZ | 2.08 | 0.04 | N | 1.75 | 1.25 | 50.97 | 59.85 | −5.45 | 3.34 | 2.11 | 0.07 |
| VH E79 | | | | | | | | | | | |
| AZ Vk 26 + AZ | 3.07 | 0.05 | N | 0.75 | 1.25 | 50.62 | 60.33 | −4.80 | 2.58 | 2.22 | 0.05 |
| VH E82 | | | | | | | | | | | |
| AZ Vk 29 + AZ | 5.64 | 0.07 | N | 1.75 | 0.75 | 49.39 | 60.46 | −6.13 | 2.83 | 3.30 | 0.07 |
| VH R75 | | | | | | | | | | | |
| AZ Vk 29 + AZ | 3.08 | 0.04 | N | 1.75 | 0.25 | 50.27 | 60.34 | −3.20 | 1.45 | 1.74 | 0.10 |
| VH R79 | | | | | | | | | | | |
| AZ Vk 29 + AZ | 2.61 | 0.04 | N | 1.75 | 0.75 | 50.09 | 60.58 | −4.43 | 1.80 | 2.63 | 0.07 |
| VH R82 | | | | | | | | | | | |
| AZ VK 29 + AZ | 2.13 | 0.03 | N | 1.75 | 1.25 | 50.62 | 60.79 | −2.18 | 0.00 | 2.18 | 0.06 |
| VH SN75 | | | | | | | | | | | |
| AZ VK 29 + AZ | 3.08 | 0.05 | N | 1.75 | 1.25 | 49.92 | 60.36 | −3.27 | 1.05 | 2.22 | 0.08 |
| VH SN79 | | | | | | | | | | | |
| AZ Vk 29 + AZ | 1.73 | 0.06 | N | 1.75 | 0.75 | 50.27 | 60.66 | −2.11 | 0.23 | 1.88 | 0.06 |
| VH SN82 | | | | | | | | | | | |
| AZ Vk 29 + AZ | 1.82 | 0.03 | N | 1.25 | 0.75 | 49.92 | 60.59 | −3.00 | 1.56 | 1.44 | 0.05 |
| VH E75 | | | | | | | | | | | |
| AZ Vk 29 + AZ | 2.12 | 0.03 | N | 1.25 | 0.75 | 50.09 | 60.21 | −3.83 | 1.65 | 2.18 | 0.07 |
| VH E79 | | | | | | | | | | | |
| AZ Vk 29 + AZ | 1.67 | 0.03 | N | 1.75 | 0.75 | 49.92 | 60.46 | −1.86 | 0.44 | 1.42 | 0.05 |
| VH E82 | | | | | | | | | | | |

Purified anti-CD3 variant-containing DuetMabs that exhibited >95% correctly-paired light chains and >98% monomer were thoroughly characterized for non-specific binding, reversible self-association, and thermal stability. To assess non-specific binding, anti-CD3 variant-based DuetMabs were evaluated using a baculovirus particle (BVP) ELISA and a human embryonic kidney 293 (HEK293) cell binding assay by flow cytometry. CD3 is neither expressed in the insect cells used to generate the baculovirus particles nor HEK293; instead, these assays assess the ‘stickiness’ of the antibody tested. Our assessment of the parental antibody showed a modest risk for non-specific binding to baculovirus particles but did not show non-specific binding to HEK293 cells. In assays assessing non-specific binding of the anti-CD3 variants, only the AZ Vκ 29+AZ VH R75 variant showed a risk for non-specific binding, where the pattern (non-specific binding to baculovirus particles but not HEK293) was similar to the parental antibody albeit at lower levels. Then, the thermal stability of anti-CD3 variants were assessed by differential scanning fluorimetry. Both the parental antibody and all anti-CD3 variants exhibited melting temperatures (Tₘ) of approximately 61° C. with the temperatures at the onset of melting (Tₒₙₛₑₜ) of ˜49-50° C.

In addition to these developability assessments, anti-CD3 variants were assessed in an accelerated stability study, where antibodies were incubated at 45° C. for 14 days and samples pre- and post-thermal stress were assessed for fragmentation and aggregation.

### Example 9—Multispecific Antibodies Containing Charge Pairs

To improve correct chain pairing beyond what alternative disulfides can achieve in DuetMab setting (see WO 2013/096291, incorporated herein by reference), charge pairs were designed using amino acids that participate in lambda light chain (LC)-heavy chain (HC) interface. The following positions were evaluated as lambda light chain amino acids participating in interface formation with CH1 domain: T117, F119, S122, E124, E125, K130, T132, V134, L136, S138, D139, E161, T163, S166, Q168, A174, S176, Y178, S180, in connection with the following heavy chain CH1 domain amino acids participating in interface formation with lambda light chain CL domain: S124, F126, L128, A129, S131, S132, K133, S134, A141, G143, L145, K147, D148, H168, F170, P171, V173, Q175, S176, S181, S183, V185, T187, V211, K213.

These amino acids were explored pairwise or alone, one pair at a time or in combinations, with alternative interchain disulfides or keeping disulfides native. Introduction of positively or partially positively charged amino acid means substituting existing amino acids at that position with lysine and arginine and in some cases with asparagine or glutamine or histidine. Introduction of negatively or partially negatively charged amino acid means substituting existing amino acids at that position with aspartic acid, glutamic acid, serine, threonine and in some cases with asparagine or glutamine. Addition of histidine residue at some of these positions will allow to introduce pH dependent CH1-CL interaction.

Nine sets of pair combinations in the lambda LC-HC interface meeting the criteria mentioned above are provided in Table 3 as a non-exhaustive example and were tested for improved pairing in this specification.

TABLE 3
All presented here mutations are specific for lambda light chain
containing molecules and expected to function as lambda charge
pairs. In addition, opposite charge pairs [i.e., V134(D, E, S,
T)-L128(R, K, H)] are also expected to provide preferential
pairing. Net no charge side chain containing amino acids like asparagine
and glutamine can be used for substitutions for either bearing
positive or negative partial charge as they have been found to
participate in formation of hydrogen bonds with both positively
and negatively charged amino acids as well as to each other.

| Set | Cλ (+) | CH1(−) |
|---|---|---|
| #1 | V134 (R, K, H) | L128 (D, E, S, T) |
| #2 | V134 (R, K, H) | L145 (D, E, S, T) |
| #3 | L136 (R, K, H) | V185 (D, E, S, T) |
| #4 | T117 (R, K, H) | V185 (D, E, S, T) |
| #5 | T117 (R, K, H) | A141 (D, E, S, T) |
| #6 | F119 (R, K, H) | L128 (D, E, S, T) |
| #7 | Y178 (R, K, H) | V173 (D, E, S, T) |
| #8 | V134 (R, K, H) | S183 (D, E, T) |
| #9 | T117 (R, K, H) | T187 (D, E) |

For construction of DuetMab antibodies with charge pair mutations in heavy chain-light chain interface, the pDuet-Heavy and pDuet-Light plasmids described in (WO 2013/096291 and in Mazor et. al mAbs 2015) were used as backbone vectors. Briefly, the pDuet-Heavy vector contained two human gamma1 heavy chain (HC) cassettes to support HC heterodimerization, where the former heavy chain carried the “Hole” set of mutations (T366S/L368A/Y407V) and a stabilizing mutation (Y349C) in CH3 domain, while the latter carried the complement “Knob” mutation (T366W) and a stabilizing mutation (S354C) in CH3, although the order of the cassettes could readily be reversed. The pDuet-Light vector contained two human light chain (LC) cassettes, where the former light chain carried a kappa constant domain (Cκ), while the latter carried a lambda constant domain (Cλ). The pDuet-Heavy and pDuet-Light vectors also contained the mutations to remove the native interchain disulfide bond in CH1/Cλ and provide the alternative disulfide bond which is denoted as “V12 DS” or “V12” in this specification, where the mutations F126C/C220V were introduced in the CH1 domain of the “Knob” heavy chain, and mutations S122C/C212V were introduced in the lambda constant domain. The amino acid sequences of the constant domains in the exemplified DuetMab antibody backbones (prior to the introduction of charge mutations) is provided as follows:

The mutations of the “Knob-and-Hole” set and the stabilizing/alternative disulfide bonds utilized herein were provided merely as an example. One skilled in the art can use any other combinations of mutations for “Knob-and-Hole” technique and/or stabilizing/alternative disulfide bonds known in this field to support HC heterodimerization.

For construction of the pDuet-Heavy vector with charge mutations, the “Hole” heavy chain was cloned into the pDuet-Heavy vector by a synthesized DNA fragment of VH-CH1-CH2-CH3 domains containing the above-mentioned mutations for “Hole” heavy chain using restriction cloning technique by BssHII/HindIII. Optionally, the “Hole” heavy chain contained the charge mutation S183K in CH1 domain. The “Knob” heavy chain was cloned into the vector by a synthesized DNA fragment of VH-CH1-CH2-CH3 domains containing the above-mentioned mutations for “Knob” heavy chain using restriction cloning technique by BsrGI/EcoRI. Optionally, the “Knob” heavy chain contained one of the charge mutations in CH1 domain: L128D, L128E, L128S, L128T, A141D, A141E, A141S, A141T, L145D, L145E, L145S, L145T, S183D, V185D, V185E, V185S, V185T, V173D, V173E, V173S, and V173T.

For construction of the pDuet-Light with charge mutations, the kappa light chain was cloned into the pDuet-Light vector by a synthesized DNA fragment of VL-Cκ domains using restriction cloning technique by BssHII/NheI. Optionally, the constant kappa (Cκ) domain contained the charge mutation V133E. The lambda light chain was cloned into the pDuet-Light vector by a synthesized DNA fragment of VL-Cλ domains containing the above-mentioned S122C/C212V mutations for lambda light chain using restriction cloning technique by BsrGI/EcoRI. Optionally, the constant lambda (Cλ) domain contained one of the charge mutations: V117R, V117K, F119R, F119K, V134R, V134K, L136R, L136K, Y178R, and Y178K. The light chain variable domain (VL) could be either variable kappa domain (Vκ) or variable lambda domain (Vλ).

All constructs were transiently expressed in CHO cells in suspension using PEI-MAX (Polysciences, Inc., Warrington, PA) as a transfection reagent and grown in an in-house made CHO medium. The vectors containing the following combinations of charge pairs were used for the expression of the antibodies in these studies. A schematic of the constructed DuetMabs containing charge pairs is provided in FIG. 20. Table 4 below provides details of various lambda charge pair DuetMabs targeting CD3 and EGFR that were produced.

TABLE 4
Details of EGFR/CD3 DuetMabs produced containing lambda charge pairs

| | pDuet-Heavy | | | | | | | |
|---|---|---|---|---|---|---|---|---|
| | | | Knob Heavy | | pDuet-Light | | | |
| | | | Chain | | Kappa Light | | Lambda Light Chain | |
| Sample | Hole Heavy Chain | | | CH1 | Chain | | | CL |
| # | Target | CH1 | Target | (V12) | Target | CL | Target | (V12) |
| 1 | EGFR | WT | CD3 | WT | EGFR | WT | CD3 | WT |
| 2 | EGFR | S183K | CD3 | WT | EGFR | V133E | CD3 | WT |
| 33 | EGFR | S183K | CD3 | A141D | EGFR | V133E | CD3 | T117R |
| 34 | EGFR | S183K | CD3 | A141E | EGFR | V133E | CD3 | T117R |
| 35 | EGFR | S183K | CD3 | A141S | EGFR | V133E | CD3 | T117R |
| 36 | EGFR | S183K | CD3 | A141T | EGFR | V133E | CD3 | T117R |
| 41 | EGFR | S183K | CD3 | A141D | EGFR | V133E | CD3 | T117K |

The culture medium was collected 7 to 13 days after transfection and filtered through a 0.22 μm sterile filter. Antibody concentration in culture supernatants was measured by an Octet384 instrument using protein A sensors (Sartorius, Göttingen, Germany) according to the manufacturer's protocol. Antibodies were purified by either protein A magnetic bead affinity purification (Genscript, Piscataway, NJ) or standard protein A affinity chromatography (Cytiva, Marlborough, MA), followed by light chain affinity chromatography if necessary, in accordance with the manufacturer's protocol, and were subsequently buffer exchanged in PBS (pH 7.2). The purity and oligomeric state of purified molecules was determined by microfluidics-based electrophoresis and analytical size exclusion chromatography (see methods below). Protein aggregates were removed by preparative SEC. The concentrations of the purified antibodies were determined by reading the absorbance at 280 nm using theoretically determined extinction coefficients.

Analytical SEC-HPLC (Agilent 1260 Infinity HPLC system) was performed using a TSK-gel G3000SWxL column (Tosoh Biosciences, King of Prussia, PA) to determine the oligomeric state of purified molecules. Preparative SEC-HPLC was carried out using a Superdex 200 column (Cytiva) to remove protein aggregates.

Microfluidics-based electrophoresis was performed using Bioanalyzer in accordance with the manufacturer's protocol (Agilent, Santa Clara, CA), in order to assess the ratio of kappa and lambda light chains of an antibody, based on which the percentage of correct light chain ratio was calculated.

Table 5 summarizes the expression and biochemical profiles of selected charge pair variants in EGFR/CD3 DuetMabs. Charge pair variants #33, #34, #35, #36, and #41 showed improved correct LC ratio compared to controls #1 and #2.

TABLE 5
Summary of expression and biochemical profiles of
selected charge pair variants in EGFR/CD3 DuetMabs.

| | | | | | | Day 10/12 | | |
|---|---|---|---|---|---|---|---|---|
| | | Hole arm | | Knob arm | | Titer | % Correct | |
| DuetMab | Sample # | CH1 | Cκ | CH1 (V12) | Cλ (V12) | (μg/mL) | LC Ratio | % Monomer |
| EGFR/CD3 | 1 | WT | WT | WT | WT | 364.2 | 38.2 | 94.8 |
| | 2 | S183K | V133E | WT | WT | 313.6 | 66.4 | 97.3 |
| | 33 | S183K | V133E | A141D | T117R | 289.9 | 89.8 | 96.5 |
| | 34 | S183K | V133E | A141E | T117R | 365.5 | 93.6 | 96.2 |
| | 35 | S183K | V133E | A141S | T117R | 262.7 | 78.4 | 197.4 |
| | 36 | S183K | V133E | A141T | T117R | 241.1 | 79.2 | 197.1 |
| | 41 | S183K | V133E | A141D | T117K | 252.7 | 92.1 | 97.1 |

### Example 10—Generation of 2+1 Bispecific Antibodies Containing Lambda Charge Pairs

Vector p2+1-Heavy was constructed on the backbone of pDuet-Heavy described in Example 9. For construction of p2+1-Heavy vector with charge mutations, the “Hole” heavy chain was cloned into the vector by BssHII/HindIII as previously described. The “Knob” heavy chain was cloned into the vector by a synthesized DNA fragment of VH-CH1-VH-CH1-CH2-CH3 domains using restriction cloning technique by BsrGI/EcoRI, where the preceding VH-CH1 segment corresponded to the sequence found on the “Hole” heavy chain, and the subsequent VH-CH1 segment contained the VH against another target as well as the charge mutation A141D and V12 DS in CH1. The pDuet-Light vector is common for both the Duet2 (2+1) Bispecific and DuetMab constructs.

All Duet2 (2+1) Bispecific constructs were transiently expressed and purified as described above for DuetMab molecules.

A schematic of the constructed Duet2 (2+1) containing charge pairs is provided in FIG. 21.

Table 6 summarizes the expression and biochemical profiles of EGFR/CD3 and NIP228/CD3 Duet2 (2+1) bispecific molecules carrying the selected sets of charge pairs produced in 500 mL cell culture. NIP228 is a non-specific control binding domain. For additional analysis, the DuetMabs were further purified by light chain affinity chromatography to remove mispaired byproducts, and aggregates were removed by preparative SEC.

TABLE 6
Production of Duet2 (2 + 1) Bispecific with charge mutations.

| | | | | | | | | | Profiles after | | | | |
|---|---|---|---|---|---|---|---|---|---|---|---|---|---|
| | | | | | | | | | purification | | Profiles of after Light chain | | |
| | | | | | | | | | protein A | | purification | | |
| | Antigen 1/ | | Antigen 1/ | | CD3 (Knob | | Titer | | Ratio of | | Ratio of | | |
| Duet2 | NIP228 (Hole | | NIP228 | | arm) | | (ug/mL) | | Kappa | | Kappa | | |
| (2 + 1) | arm) | | (Knob arm) | | CH1 | Cλ | Day | Day | and | % | and | % | Endotoxin |
| Bispecific | CH1 | C_{K} | CH1 | C_{K} | (V12) | (v12) | 7 | 14 | Lambda | Monomer | Lambda | Monomer | (EU/mg) |
| EGFR/CD3 | S183K | V133E | S183K | V133E | A141D | T117R | 55 | 130 | 2.49:1 | 90 | 2:1 | >99 | <0.5 |
| 2 + 1 | | | | | | | | | | | | | |
| NIP228/ | S183K | V133E | S183K | V133E | A141D | T117R | 96 | 380 | 2.39:1 | 91 | 1.9:1 | >99 | <0.5 |
| CD3 2 + 1 | | | | | | | | | | | | | |

Table 7 summarizes the thermal stabilities of EGFR/CD3 and NIP228/CD3 Duet2 (2+1) Bispecific molecules by differential scanning fluorimetry (DSF) and their accelerated stability profiles. The Duet2 (2+1) Bispecific molecules showed no flags for aggregation or fragmentation after heat stress.

TABLE 7
Developability Summary of Duet2 (2+1)
Bispecific with Charge Mutations

| | Accelerated Stability (45° C., 14 d) | | | | | |
|---|---|---|---|---|---|---|
| | | | | ΔRT | | |
| Duet2 | Monomer | Aggregate | Fragment | from | | |
| (2 + 1) | Loss | Increase | Increase | NIP228 | DSF | |
| Bispecific | (%) | (%) | (%) | (m) | Tₒₙₛₑₜ | Tₘ |
| EGFR/CD3 | 6.18 | 0.30 | 5.87 | 6.18 | 50 | 60.6 |
| 2 + 1 | | | | | | |
| NIP228/ | 3.19 | 0.69 | 2.49 | 3.19 | 47 | 58.3 |
| CD3 2 + 1 | | | | | | |

Table 8 shows the sub-unit mass spectrum data of EGFR/CD3 and NIP228/CD3 Duet2 (2+1) Bispecific molecules. The alignment of theoretical mass and measured mass confirmed molecule integrity and LC/HC association identity of each variant.

TABLE 8
MS results of sub-unit LC/MS analysis.

| EGFR/ | measured | theoretical |
|---|---|---|
| CD3 2 + 1 | mass | mass |
| Fc with 2 G0F | 53095 | 53095 |
| Antigen 1 LC + Antigen 1 HC Fab (Hole arm) | 47545 | 47544 |
| Antigen 1 LC + Antigen 1 HC Fab + CD3 | 95876 | 95876 |
| LC + CD3 HC Fab (Knob arm) | | |

| NIP 228/ | measured | theoretical |
|---|---|---|
| CD3 2 + 1 | mass | mass |
| Fc with 2 G0F | 53095 | 53095 |
| NIP228-LC + NIP228 HC Fab (Hole arm) | 46991 | 46991 |
| NIP228 LC + NIP228 HC Fab + CD3 | 95322 | 95322 |
| LC + CD3 HC Fab (Knob arm) | | |

Table 9 show the thermal stability studies of EGFR/CD3 and NIP228/CD3 Duet2 (2+1) Bispecific molecules using differential scanning calorimetry (DSC) analysis. Table 14 lists the deconvoluted TM and approximated Tonset values of Duet2 (2+1) Bispecific molecules.

TABLE 14
MS results of sub-unit LC/MS analysis.

| | T_{M1} | T_{M2} | T_{M3} | Approximated Tₒₙₛₑₜ |
|---|---|---|---|---|
| Name | (° C.) | (° C.) | (° C.) | (° C.) ± S.D. |
| EGFR/CD3 Duet2 (2:1) | 64.6 | 70.5 | 79.0 | 51.8 ± 0.4 |
| NIP228/CD3 Duet2 (2:1) | 66.7 | 71.8 | 79.3 | 53 ± 1.3 |

The EGFR/CD3 Duet2 (2+1) bispecific molecules were tested in a cytotoxicity assay containing T cells and EGFR-expressing target cells and activated CD8 and CD4 T cells, demonstrating that this Duet2 (2+1) is functional.

### Example 11—Generation of Trispecific Antibodies Containing Lambda and Kappa Charge Pairs

Vector pTriMab-Heavy was constructed on the backbone of pDuet-Heavy described in Example 9 above. For construction of pTriMab-Heavy vector with charge mutations, the “Hole” heavy chain was cloned into the vector by BssHII/HindIII as previously described, where the VH-CH1 segment in the “Hole” heavy chain was defined as “Fab1” with the VH against a first target and the charge mutation S183K in CH1. The “Knob” heavy chain was cloned into the vector by a synthesized DNA fragment of VH-CH1-VH-CH1-CH2-CH3 domains using restriction cloning technique by BsrGI/EcoRI. The preceding VH-CH1 segment of “Knob” heavy chain was defined as “Fab2” with the VH against a second target as well as the charge mutation S183E and optionally the V12 DS in CH1. The subsequent VH-CH1 segment of “Knob” heavy chain was defined as “Fab3” with the VH against a third target as well as the charge mutation A141D and the V12 DS in CH1.

Vectors pTriMab-Light1 and pTriMab-Light2 were constructed on the backbone of pDuet-Light described in Example 1 above. For construction of the pTriMab-Light1 with charge mutation, the kappa light chain for Fab1 was cloned into the vector by BssHII/NheI as previously described, where the Cκ domain contained the charge mutation V133E. The second (lambda) LC cassette in pTriMab-Light1 was removed.

For construction of the pTriMab-Light2 with charge mutations, the kappa light chain for Fab2 was cloned into the vector by BssHII/NheI as previously described, where the Cκ domain contained the charge mutation V133K and optionally the V12 DS (S121C/C214V for Cκ). The lambda light chain for Fab3 was cloned into the vector by BsrGI/EcoRI as previously described, where the Cλ domain contained the charge mutation T117R and the V12 DS.

All TriMab constructs were transiently expressed and purified as described above for DuetMab molecules. A schematic of the constructed TriMabs containing charge pairs is provided in FIG. 22.

Table 15 summarizes the expression and biochemical profiles of EGFR/Her2/CD3 TriMab carrying the selected sets of charge pairs produced in 500 mL cell culture. For additional analysis, the TriMabs were further purified by protein A affinity chromatography, and aggregates were removed by preparative CHT column (an incompressible mixed-mode chromatography medium using cation exchange and calcium-affinity interactions).

TABLE 15
EGFR/Her2/CD3 TriMab trispecific with charge mutations. Ratio of kappa and lambda were calculated by band density from
capillary gel electrophoresis under reducing conditions using Agilent Protein 80 Chip. Monomer content was calculated
by analytical size-exclusion chromatogram of intact. CHT ceramic hydroxyapatite is a purification mixed-mode method.

| | | | | | | | | Profiles after protein A | | Profiles of after | |
|---|---|---|---|---|---|---|---|---|---|---|---|
| | | | | | Fab3-CD3 | | | purification | | CHT | |
| | Fab1-HER2 | | Fab2-EGFR- | | (Knob arm) | | Titer | Ratio of | | Ratio of | |
| | (Hole arm) | | (knob arm) | | CH1 | Cλ | (μg/mL) | Kappa and | 1% | Kappa and | 1% |
| TriMab | CH1 | C_{K} | CH1(v12) | C_{K}(v12) | (v12) | (v12) | Day 12 | Lambda | Monomer | Lambda | Monomer |
| EGFR/HER2 | S183K | V133E | S183E | V133K | A141D | T117R | 176 | 2.12 | 95 | 2.41 | >99 |
| CD3 TriMab | | | | | | | | | | | |

Table 16 summarizes the thermal stabilities of the EGFR/Her2/CD3 TriMab by differential scanning fluorimetry (DSF) and their accelerated stability profiles. The EGFR/Her2/CD3 TriMab molecule showed no flags for aggregation or fragmentation after heat stress.

TABLE 16
Developability summary of EGFR/Her2/CD3 TriMab with
charge mutations. Ratio of kappa and lambda was calculated
by band density from capillary gel electrophoresis
under reducing conditions using Agilent Protein 80
Chip. Monomer content was calculated by analytical
size-exclusion chromatogram of intact.

| | Accelerated stability heat (45° C., 14 d) | | | DSF | |
|---|---|---|---|---|---|
| Molecule | Monomer | Aggregate | Fragment | | Tₒₙₛₑₜ |
| name | loss (%) | increase(%) | increase (%) | Tₘ(° C.) | (° C.) |
| EGFR/HER2 | 2.21 | 0 | 2.21 | 60.6 | 54.1 |
| CD3 TriMab | | | | | |

Table 17 reports the sub-unit mass spectrum data of the EGFR/Her2/CD3 TriMab. The alignment of theoretical mass and measured mass confirmed molecule integrity and LC/HC association identity of each peak.

TABLE 17
MS results of sub-unit LC/MS analysis
of EGFR/Her2/CD3 TriMab.

| | Measured | Theoretical | |
|---|---|---|---|
| peak | Mass (Da) | Mass (Da) | Annotation |
| 1 | 23588 | 23589 | Antigen 2 LC + Cys |
| 2 | 53096 | 53096 | Fc (2 × G0F) |
| | 53126 | 53128 | Fc (2 × G0F) + Trisulfide |
| 3 | 50205 | 50205 | Fc (non-glycan) |
| | 51650 | 51650 | Fc (1 × G0F) |
| 4 | 100680 | 100677 | Fab(Antigen 2) + Fc |
| | 100710 | 100709 | Fab(Antigen 2) + Fc + Trisulfide |
| 5 | 47599 | 47599 | Fab(Antigen 2) |
| 6 | 148953 | 148951 | Fab(Antigen 1-CD3) + Fc + Trisulfide |
| 7 | 95842 | 95841 | Fab(Antigen 1-CD3) |

Table 18 reports the thermal stability studies of the EGFR/Her2/CD3 TriMab using differential scanning calorimetry (DSC) analysis and lists the deconvoluted TM and approximated Tonset values of the EGFR/Her2/CD3 TriMab.

TABLE 18
DSC thermostability measurements captured transitions
for the Fab, CH1, CH2, and CH3 domains under the Tₘ1, Tₘ2,
and Tₘ3 descriptions. Some transitions are summated
in the same Tm peak transition

| | | | | | Approximated |
|---|---|---|---|---|---|
| | T_{M}1 | T_{M}2 | T_{M}3 | Total Enthalpy | Tonset |
| Name | (° C.) | (° C.) | (° C.) | (kcal/mol) FIO | (° C.) ± S.D. |
| EGFR/HER2 | 64.0 | 68.3 | 77.0 | 1549.74 | 49.6 ± 3.1 |
| CD3 TriMab | | | | | |

The EGFR/Her2/CD3 TriMab molecules were tested in a cytotoxicity assay containing T cells and cells positive for EGFR only (“single-positive” cells) or positive for both EGFR and HER2 (“double-positive” cells). The EGFR/Her2/CD3 TriMab showed cell killing, CD8 and CD4 T cell activation of both single-positive and double-positive cells, demonstrating that this TriMab format is functional.

1. An antibody comprising an antigen binding domain that is capable of binding to a CD3 protein or a fragment thereof, wherein the CD3 antigen binding domain comprises a heavy chain variable (VH) region according to any one of the following:
a VH region comprising the following CDRs:
HCDR1 having the amino acid sequence of SEQ ID NO: 90;
HCDR2 having the amino acid sequence of SEQ ID NO: 91; and
HCDR3 having the amino acid sequence of SEQ ID NO: 92,
a VH region comprising the following CDRs:
HCDR 1 having the amino acid sequence of SEQ ID NO: 82;
HCDR2 having the amino acid sequence of SEQ ID NO: 83; and
HCDR3 having the amino acid sequence of SEQ ID NO: 84,
a VH region comprising the following CDRs:
HCDR 1 having the amino acid sequence of SEQ ID NO: 78;
HCDR2 having the amino acid sequence of SEQ ID NO: 79; and
HCDR3 having the amino acid sequence of SEQ ID NO: 80,
a VH region comprising the following CDRs:
HCDR1 having the amino acid sequence of SEQ ID NO: 66;
HCDR2 having the amino acid sequence of SEQ ID NO: 67; and
HCDR3 having the amino acid sequence of SEQ ID NO: 68,
a VH region comprising the following CDRs:
HCDR1 having the amino acid sequence of SEQ ID NO: 70;
HCDR2 having the amino acid sequence of SEQ ID NO: 71; and
HCDR3 having the amino acid sequence of SEQ ID NO: 72,
a VH region comprising the following CDRs:
HCDR 1 having the amino acid sequence of SEQ ID NO: 74;
HCDR2 having the amino acid sequence of SEQ ID NO: 75; and
HCDR3 having the amino acid sequence of SEQ ID NO: 76,
a VH region comprising the following CDRs:
HCDR 1 having the amino acid sequence of SEQ ID NO: 86;
HCDR2 having the amino acid sequence of SEQ ID NO: 87; and
HCDR3 having the amino acid sequence of SEQ ID NO: 88,
a VH region comprising the following CDRs:
HCDR 1 having the amino acid sequence of SEQ ID NO: 94;
HCDR2 having the amino acid sequence of SEQ ID NO: 95; and
HCDR3 having the amino acid sequence of SEQ ID NO: 96, and
a VH region comprising the following CDRs:
HCDR1 having the amino acid sequence of SEQ ID NO: 98;
HCDR2 having the amino acid sequence of SEQ ID NO: 99; and
HCDR3 having the amino acid sequence of SEQ ID NO: 100,
and wherein the CD3 antigen binding domain comprises a light chain variable (VL) region according to any one of the following:
a VL region comprising the following CDRs:
HCDR1 having the amino acid sequence of SEQ ID NO: 46;
HCDR2 having the amino acid sequence of SEQ ID NO: 47; and
HCDR3 having the amino acid sequence of SEQ ID NO: 48,
a VL region comprising the following CDRs:
HCDR1 having the amino acid sequence of SEQ ID NO: 58;
HCDR2 having the amino acid sequence of SEQ ID NO: 59; and
HCDR3 having the amino acid sequence of SEQ ID NO: 60, and
a VL region comprising the following CDRs:
HCDR 1 having the amino acid sequence of SEQ ID NO: 62;
HCDR2 having the amino acid sequence of SEQ ID NO: 63; and
HCDR3 having the amino acid sequence of SEQ ID NO: 64.

2. The antibody according to claim 1, wherein the CD3 antigen binding domain comprises a VH region and VL region comprising one of the following sets of CDRs:
or
i. HCDR1 having the amino acid sequence of SEQ ID NO: 90,
ii. HDCR2 having the amino acid sequence of SEQ ID NO: 91,
iii. HCDR3 having the amino acid sequence of SEQ ID NO: 92,
iv. LCDR1 having the amino acid sequence of SEQ ID NO: 46,
v. LCDR2 having the amino acid sequence of SEQ ID NO: 47
vi. LCDR3 having the amino acid sequence of SEQ ID NO: 48; or
vii. HCDR1 having the amino acid sequence of SEQ ID NO: 82,
viii. HDCR2 having the amino acid sequence of SEQ ID NO: 83,
ix. HCDR3 having the amino acid sequence of SEQ ID NO: 84,
x. LCDR1 having the amino acid sequence of SEQ ID NO: 46,
xi. LCDR2 having the amino acid sequence of SEQ ID NO: 47,
xii. LCDR3 having the amino acid sequence of SEQ ID NO: 48; or
xiii. HCDR1 having the amino acid sequence of SEQ ID NO: 78
xiv. HDCR2 having the amino acid sequence of SEQ ID NO: 79
xv. HCDR3 having the amino acid sequence of SEQ ID NO: 80,
xvi. LCDR1 having the amino acid sequence of SEQ ID NO: 62,
xvii. LCDR2 having the amino acid sequence of SEQ ID NO: 63,
xviii. LCDR3 having the amino acid sequence of SEQ ID NO: 64,

3. The antibody according to claim 1, wherein the CD3 antigen binding domain comprises a VH region having at least 90%, 91%, 92%, 93%, 94%, 95%, 96%, 97%, 98%, 99%, or 100% sequence identity to SEQ ID NO: 77, SEQ ID NO: 89, or SEQ ID NO: 81.

4. The antibody according to claim 1, wherein the CD3 antigen binding domain comprises a VL region having at least 90%, 91%, 92%, 93%, 94%, 95%, 96%, 97%, 98%, 99%, or 100% sequence identity to SEQ ID NO: 45, or SEQ ID NO: 61.

5. (canceled)

6. The antibody according to claim 1, wherein the CD3 antigen binding domain binds to human CD3 with an affinity having a K_{d} that is:
i. between 10-fold and 200-fold higher;
ii. between 15-fold and 200-fold higher;
iii. between 20-fold and 200-fold higher; or
iv. between 25-fold and 200 fold higher
compared to the K_{d} of a control antigen binding domain binding to human CD3, wherein the control antigen domain binding has a VH domain sequence of SEQ ID NO: 5 and a VL domain sequence of SEQ ID NO: 1.

7. The antibody according to claim 1, wherein the CD3 antigen binding domain exhibits reduced off-target T cell activity as compared to a control antigen binding domain, wherein the control antigen domain binding has a VH domain sequence of SEQ ID NO: 5 and a VL domain sequence of SEQ ID NO: 1, optionally wherein off-target T cell activation is determined in a T cell activation assay in the absence of engagement with a target cell.

8. The antibody according to claim 1, further comprising a target antigen binding domain, and wherein the target antigen binding domain is capable of binding to a tumor associated antigen (TAA).

9. (canceled)

10. The antibody according to claim 8, wherein the TAA is selected from the list consisting of AFP, aₙb3 (vitronectin receptor), aₙb₆, B-cell maturation agent (BCMA), CA125 (MUC16), CD4, CD20, CD22, CD33, CD52, CD56, CD66e, CD80, CD140b, CD227 (MUC1), EGFR (HER1), EpCAM, GD3 ganglioside, HER2, prostate-specific membrane antigen (PSMA), prostate specific antigen (PSA), CD5, CD19, CD21, CD25, CD37, CD30, CD33, CD45, HLA-DR, anti-idiotype, carcinoembryonic antigen (CEA), e.g. carcinoembryonic antigen-related cell adhesion molecule 5 (CEACAM5), TAG-72, Folate-binding protein, A33, G250, ferritin, glycolipids such as gangliosides, carbohydrates such as CA-125, IL-2 receptor, fibroblast activation protein (FAP), IGF1 R, B7H3, B7H4, PD-L1, CD200, EphA2, c-Met, and mesothelin.

11. The antibody according to claim 10, wherein the TAA is EGFR, HER2, STEAP2, GPC3, and c-Met.

12. The antibody according to claim 7, wherein one of the antigen binding domains comprises a CH1 and a lambda constant (CLλ) region, optionally wherein the CD3 antigen binding domains comprises the CH1 and CLλ region.

13. The antibody according to claim 12, wherein the CLλ has at least 90%, 91%, 92%, 93%, 94%, 95%, 96%, 97%, 98%, 99% to SEQ ID NO: 105.

14. The antibody according to claim 12, wherein the antigen binding domains comprising the CLλ region comprises a lambda charge pair, optionally wherein lambda charge pair is located at one or more of the following pairs of positions in the antigen binding domain:
i. position 117 in the CLλ and position 141 in the CH1;
ii. position 117 in the CLλ and position 185 in the CH1;
iii. position 119 in the CLλ and position 128 in the CH1;
iv. position 134 in the CLλ and position 128 in the CH1;
v. position 134 in the CLλ and position 145 in the CH1;
vi. position 134 in the CLλ and position 183 in the CH1;
vii. position 136 in the CLλ and position 185 in the CH1;
viii. position 178 in the CLλ and position 173 in the CH1; and
vx. position 117 in the CLλ and position 187 in the CH1,
wherein the lambda charge pair comprises a positively charged amino acid residue optionally selected from arginine, lysine or histidine located at one of the positions in the lambda charge pair and a negatively charged amino acid residue optionally selected from aspartic acid, glutamic acid, serine or threonine located at the other position in the lambda charge pair, and
wherein the numbering is according to the EU index.

15. The antibody according to claim 14, wherein the lambda charge pair is located at position 117 in the CLλ and position 141 in the CH1, optionally wherein the lambda charge pair is selected from the following list:
a. arginine at position 117 of the CLλ and aspartic acid at position 141 of the CH1;
b. arginine at position 117 of the CLλ and glutamic acid at position 141 of the CH1;
c. arginine at position 117 of the CLλ and serine at position 141 of the CH1;
d. arginine at position 117 of the CLλ and threonine at position 141 of the CH1; and
e. lysine at position 117 of the CLλ and aspartic acid at position 141 of the CH1.

16. The antibody according to claim 15, wherein the other antigen binding domain comprises a CH1 and a kappa constant CLκ region, optionally wherein the target antigen binding domains comprises the CH1 and CLκ region.

17. The antibody according to claim 16, wherein the CLκ has at least 90%, 91%, 92%, 93%, 94%, 95%, 96%, 97%, 98%, 99% to SEQ ID NO: 106.

18. The antibody according to claim 17, wherein the antigen binding domains comprising the CLκ region comprises a kappa charge pair, optionally wherein kappa charge pair is located at position 133 in the CLκ and position 183 in the CH1.

19. The antibody according to claim 8, wherein either:
xix. the disulfide link between the light chain and CH1 in the CD3 antigen binding arm is formed between a pair of cysteines engineered into the light chain and the CH1 of the CD3 antigen binding arm, and the disulfide link between the light chain and CHI in the target antigen binding arm is formed between a pair of native cysteines; or
xx. the disulfide link between the light chain and CHI in the target antigen binding arm is formed between a pair of cysteines engineered into the light chain and the CHI of the target antigen binding arm, and the disulfide link between light chain and CHI in the CD3 antigen binding arm is formed between a pair of native cysteines.

20. The antibody according to claim 19, wherein the pair of cysteines engineered into the light chain and CH1 of the CD3 antigen binding arm are located at position 122 of the light chain and position 126 of the CH1 of the CD3 antigen binding arm, and wherein the light chain of the CD3 antigen binding arm comprises a non-cysteine residue at position 212 and the CH1 of the CD3 antigen binding arm comprises a non-cysteine residue at position 220, optionally wherein the non-cysteine residues are valines.

21-24. (canceled)

25. The antibody according to claim 1, comprising modifications in the CH3 of the Fc regions, wherein a substitution to generate a knob is a substitution to tryptophan at position 366 and the substitution to generate a hole is a substitution to generate a hole is one or more of the following:
i. a substitution to valine at position 407;
ii. a substitution to serine at position 366; and
xxi. a substitution to alanine at position 368.

26. The antibody according to claim 25, wherein the CH3 domain containing the protuberance (knob) comprises a cysteine at position 354 and the CH3 domain containing the cavity (hole) comprises a cysteine at position 349.

27. The antibody according to claim 26, wherein at least one of the Fc regions comprises the amino acid substitutions:
a. L234F/L235E/P331S;
b. E233P/L234V/L235A/G236del/S267K; and/or
c. M252Y/S254T/T256E.

28. The antibody according to claim 27, comprising two antigen binding domains that are capable of binding the same target.

29. The antibody according to claim 28, further comprising a CD8 antigen binding domain, optionally wherein the CD8 antigen binding domain is a VHH and comprises either
(1) the following complementarity determining regions (CDRs):
HCDR 1 having the amino acid sequence of SEQ ID NO: 109, 116 or 117;
HCDR2 having the amino acid sequence of SEQ ID NO: 110; and
HCDR3 having the amino acid sequence of SEQ ID NO: 111; or
(2) comprises a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 112, 118, 119 or 120.

30. The antibody according to claim 8, further comprising a third antigen binding domain, optionally wherein the third antigen binding domain binds to CD8, or binds to a different target than the target antigen binding domain.

31. One or more nucleic acid(s) encoding the antibody according to claim 1.

32. A vector comprising the nucleic acid(s) of claim 31.

33. An isolated host cell comprising the nucleic acid(s) of claim 31.

34-35. (canceled)

36. A pharmaceutical composition comprising the antibody according to claim 1 and a pharmaceutically acceptable carrier.

37. A method of treating a disease in a patient in need thereof, the method comprising administering to the patient an effective amount of the antibody according to claim 1.

38. The method of claim 37, wherein the disease is cancer.

39. The antibody according to claim 1, for use as a medicament, optionally wherein the antibody is a multispecific antibody.

40. The antibody according to claim 1, for use in the treatment of cancer, optionally wherein the antibody is a multispecific antibody.

41. Use of the antibody according to claim 1, for the manufacture of a medicament for the treatment of cancer, optionally wherein the antibody is a multispecific antibody.
